SEQUENCE LISTING (132 sequences)

SEQ ID NO: 1 - protein, 736 aa, Artificial Sequence
  Adeno-associated virus
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 2 - protein, 736 aa, Artificial Sequence
  Adeno-associated virus
MTADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKQLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 3 - protein, 736 aa, Artificial Sequence
  Adeno-associated virus
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTGGNFHPSPLMGGFGMKHPPPQILIKNTPVPADPPTAFNKDKLNSFITQYSTGQVSVEIEWELQKENSKRWNPEIQYTSNYYKSNNVEFAVNTGGVYSEPRPIGTRYLTRNL

SEQ ID NO: 4 - protein, 736 aa, Artificial Sequence
  Adeno-associated virus
MAADGYLPDWLEDNLSEGIREWWALKPGAPQPKANQQHQDNARGLVLPGYKYLGPGNGLDKGEPINAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTYGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 5 - protein, 736 aa, Artificial Sequence
  Adeno-associated virus
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIDKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 6 - protein, 736 aa, Artificial Sequence
  Adeno-associated virus
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVLQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGSSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 7 - protein, 736 aa, Artificial Sequence
  Adeno-associated virus
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQARAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 8 - protein, 736 aa, Artificial Sequence
  Adeno-associated virus
MAADGYLPDWLEDNLSEGIREWWALKPGAPQPKANQQHQDNARGLVLPGYKYLGPGNGLDKGEPVNAVDAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 9 - protein, 736 aa, Artificial Sequence
  Adeno-associated virus
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPREPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 10 - protein, 736 aa, Artificial Sequence
  Adeno-associated virus
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASCGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 11 - protein, 736 aa, Artificial Sequence
  Adeno-associated virus
MAADGYLPDWLEDNLSEGIREWWALKPGAPQPKANQQHQDNARGLVLPGYKYLGPGNGLDKGEPVNAADAAALEHDRAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKKNSKRWNPEIQYTSNYYKSNNVEFAVNTEGVYSEPRPIGTRYLTRNL

SEQ ID NO: 12 - protein, 736 aa, Artificial Sequence
  Adeno-associated virus
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPHDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFNVAGPSNMAVQGRNYIPGPSYRQQRVSTTVTQNNNSEFAWPRASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSMEIEWELQKENSKRWNPEIQYTSNYYKSNNVEFAVNTEGVYSEPRPIGTRYLTRNL

SEQ ID NO: 13 - protein, 736 aa, Artificial Sequence
  Adeno-associated virus
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPRASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 14 - DNA, 145 bp, Artificial Sequence
  Synthetic nucleic acid
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 15 - protein, 736 aa, Artificial Sequence
  Adeno-associated virus
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPRASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWERQKENSKRWNPEIQYTSNYYKSNNVEFAVNTEGVYSEPRPIGTRYLTRNL

SEQ ID NO: 16 - protein, 736 aa, Artificial Sequence
  Adeno-associated virus
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFADSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPRASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 17 - protein, 736 aa, Artificial Sequence
  Adeno-associated virus
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEIAWPRASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYCKSNNVEFAVNTEGVYSEPRPIGTRYLTRNL

SEQ ID NO: 18 - DNA, 145 bp, Artificial Sequence
  Synthetic nucleic acid
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 19 - DNA, 106 bp, Artificial Sequence
  Synthetic nucleic acid
ctgcgcgctcgctcgctcactgaggccgcccgggcaaagcccgggcgtcgggcgacctttggtcgcccggcctcagtgagcgagcgagcgcgcagagagggagtgg

SEQ ID NO: 20 - DNA, 167 bp, Artificial Sequence
  Synthetic nucleic acid
ctctcccccctgtcgcgttcgctcgctcgctggctcgtttgggggggtggcagctcaaagagctgccagacgacggccctctggccgtcgcccccccaaacgagccagcgagcgagcgaacgcgacaggggggagagtgccacactctcaagcaagggggttttgta

SEQ ID NO: 21 - DNA, 167 bp, Artificial Sequence
  Synthetic nucleic acid
tacaaaacctccttgcttgagagtgtggcactctcccccctgtcgcgttcgctcgctcgctggctcgtttgggggggtggcagctcaaagagctgccagacgacggccctctggccgtcgcccccccaaacgagccagcgagcgagcgaacgcgacaggggggagag

SEQ ID NO: 22 - protein, 621 aa, adeno-associated virus 2
MPGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 23 - DNA, 57 bp, Artificial Sequence
  Synthetic nucleic acid
atgggctggtcctgcatcatcctgttcctggtggccaccgccacaggcgtgcacagc

SEQ ID NO: 24 - DNA, 66 bp, Artificial Sequence
  Synthetic nucleic acid
atggacatgagggtccctgctcagctgctggggctcctgctgctctggctcagcggtgccagatgt

SEQ ID NO: 25 - DNA, 192 bp, Artificial Sequence
  Synthetic nucleic acid
ccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggg

SEQ ID NO: 26 - DNA, 255 bp, Artificial Sequence
  Synthetic nucleic acid
aatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatc

SEQ ID NO: 27 - DNA, 448 bp, Artificial Sequence
  Synthetic nucleic acid
ccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatc

SEQ ID NO: 28 - DNA, 54 bp, Artificial Sequence
  Synthetic nucleic acid
gaaggcagaggcagcctgctgacctgcggcgacgtcgaagagaaccccggccct

SEQ ID NO: 29 - DNA, 93 bp, Artificial Sequence
  Synthetic nucleic acid
ctctaaggtaaatataaaatttttaagtgtataatgtgttaaactactgattctaattgtttctctcttttagattccaacctttggaactga

SEQ ID NO: 30 - DNA, 92 bp, Artificial Sequence
  Synthetic nucleic acid
aagaggtaagggtttaagggatggttggttggtggggtattaatgtttaattacctggagcacctgcctgaaatcactttttttcaggttgg

SEQ ID NO: 31 - DNA, 198 bp, Artificial Sequence
  Synthetic nucleic acid
gatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaa

SEQ ID NO: 32 - DNA, 143 bp, Artificial Sequence
  Synthetic nucleic acid
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcc

SEQ ID NO: 33 - DNA, 171 bp, Artificial Sequence
  Synthetic nucleic acid
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagg

SEQ ID NO: 34 - DNA, 122 bp, Artificial Sequence
  Synthetic nucleic acid
aacttgtttattgcagcttataatggttacaaataaagcaatagcatcacaaatttcacaaataaagcatttttttcactgcattctagttgtggtttgtccaaactcatcaatgtatctta

SEQ ID NO: 35 - DNA, 133 bp, Artificial Sequence
  Synthetic nucleic acid
tgctttatttgtgaaatttgtgatgctattgctttatttgtaaccattataagctgcaataaacaagttaacaacaacaattgcattcattttatgtttcaggttcagggggaggtgtgggaggttttttaaa

SEQ ID NO: 36 - DNA, 1676 bp, Artificial Sequence
  Synthetic nucleic acid
ggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacag

SEQ ID NO: 37 - DNA, 12 bp, Artificial Sequence
  Synthetic nucleic acid
agaaagagaaga

SEQ ID NO: 38 - DNA, 12 bp, Artificial Sequence
  Synthetic nucleic acid
agaagaaagaga

SEQ ID NO: 39 - DNA, 1168 bp, Artificial Sequence
  Synthetic nucleic acid
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctccagtacgtgattcttgatcccgagctggagccaggggcgggccttgcgctttaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaggatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctccagggggctcaaaatggaggacgcggcgctcgggagagcgggcgggtgagtcacccacacaaaggaaaggggcctttccgtcctcagccgtcgcttcatgtgactccacggagtaccgggcgccgtccaggcacctcgattagttctggagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 40 - DNA, 72 bp, Artificial Sequence
  Synthetic nucleic acid
agatctggcagcggagagggcagaggaagtcttctaacatgcggtgacgtggaggagaatcccggccctagg

SEQ ID NO: 41 - DNA, 95 bp, Artificial Sequence
  Synthetic nucleic acid
cctctgctaaccatgttcatgccttcttctttttcctacagctcctgggcaacgtgctggttattgtgctgtctcatcattttggcaaagaattc

SEQ ID NO: 42 - DNA, 1873 bp, Artificial Sequence
  Synthetic nucleic acid
gatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaatt

SEQ ID NO: 43 - DNA, 370 bp, Artificial Sequence
  Synthetic nucleic acid
gggggaggctgctggtgaatattaaccaaggtcaccccagttatcggaggagcaaacaggggctaagtccacctcgagccatggcgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagcgccggcaagaggtaagggtttaagggatggttggttggtggggtattaatgtttaattacctggagcacctgcctgaaatcactttttttcaggttggaccggtgccacc

SEQ ID NO: 44 - DNA, 913 bp, Artificial Sequence
  Synthetic nucleic acid
ggcatcctaaaaaatattcagtggaaacgtaaaaacattaaagactgattaaacatcgcagcatgacacagatttagcaactgagcataaataatttgactcggatactgctccaaaatccgaagaggaccaatttcttccaggaggacaactacctcgtcctctgcagacccctctcctcggcagctgaaggagtgtggccaatctgcctccacctccccgcggaccccctactctcaggacctcctgcagcaccccaaactggaagtggccgctgcagacccaaggacgaggggcacgcgggagccggcagccctagtggagcggttggagatgttgaggtgggagggtcacccaggtggggtgaggctggggtaggtagcggagtgaacggcttccgaagctctgggccgcccccaggttggactaagcaggcgctctgtcttcgcccccgcccagggtgggcgtctcctgaggactccccgccacacctgacccgagaccgcgcgcccagcctagaacgcttccccgacccagcgtagggccgccgcgactggcgggcgagggtcggcgggaggcctggcgaacccgggggcgggaccaggcgggcaaggcccggctgccgcagcgccgctctgcgcgaggcggctccgccgcggcggagggatacggcgcaccatatatatatcgcggggcgcagactcgcgctccggcagtggtgctgggagtgtcgtggacgccgtgccgttactcgtagtcaggcggcggcgcaggcggcggcggcggcatagcgcacagcgcgccttagcagcagcagcagcagcagcggcatcggaggtacccccgccgtcgcagcccccgcgctggtgcagccaccctcgctccctctgctcttcctcccttcgctcgcacc

SEQ ID NO: 45 - DNA, 137 bp, Artificial Sequence
  Synthetic nucleic acid
aggtaagtatcaaggttacaagacaggtttaaggagaccaatagaaactgggcttgtcgagacagagaagactcttgcgtttctgataggcacctattggtcttactgacatccactttgcctttctctccacaggt

SEQ ID NO: 46 - DNA, 380 bp, Artificial Sequence
  Synthetic nucleic acid
ggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatg

SEQ ID NO: 47 - DNA, 1246 bp, Artificial Sequence
  Synthetic nucleic acid
tcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcgg

SEQ ID NO: 48 - DNA, 1061 bp, Artificial Sequence
  Synthetic nucleic acid
tagggaggtcctgcacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcgggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactaaaacaggtaagtccggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgatgcctctactaaccatgttcatgttttctttttttttctacaggtcctgggtgacgaacag

SEQ ID NO: 49 - DNA, 953 bp, Artificial Sequence
  Synthetic nucleic acid
aattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaag

SEQ ID NO: 50 - DNA, 541 bp, Artificial Sequence
  Synthetic nucleic acid
ccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcctctaaggtaaatataaaatttttaagtgtataatgtgttaaactactgattctaattgtttctctcttttagattccaacctttggaactga

SEQ ID NO: 51 - DNA, 612 bp, Artificial Sequence
  Synthetic nucleic acid
gcccccttttgcatccagtttattcctacatttgtcacactgttaacagcccaccccttccaatgagaccagtggtatcagtgagttgtggagatcaggaaaagggctcaagagaaaggcagtcaaagccctttttctgtccctgtcccagctgctttaataagatctccataagagaagagggacagctatgactgggagtagtcaggagaggaggaaaaatctggctagtaaaacatgtaaggaaaattttagggatgttaaagaaaaaaataacacaaaacaaaatataaaaaaaatctaacctcaagtcaaggcttttctatggaataaggaatggacagcagggggctgtttcatatactgatgacctctttatagccaacctttgttcatggcagccagcatatgggcatatgttgccaaactctaaaccaaatactcattctgatgttttaaatgatttgccctcccatatgtccttccgagtgagagacacaaaaaattccaacacactattgcaatgaaaataaatttcctttattagccagaagtcagatgctcaaggggcttcatgatgtccccataatttttggcagagggaaaaagatc

SEQ ID NO: 52 - DNA, 1347 bp, Artificial Sequence
  Synthetic nucleic acid
caagtgcagctggttcaaagcggagccgaagtgaaaaagcccggagctagcgtgaaggtgtcctgcaaggccagcggccacatcttcagcaactactggatccagtgggtgcggcaggcccctggccaaggcctggaatggatgggcgagatcctgccaggatctggccacaccgagtacacagagaacttcaaggatagagtgaccatgaccagagatacctccacaagcaccgtgtacatggaactgagcagcctgagaagcgaggacacagctgtgtactactgcgccagatacttttttggctcatcccctaactggtacttcgacgtgtggggccaaggcacccttgtcaccgtcagcagcgccagtactaagggacccagcgtgttcccactggccccatgcagcagaagcacatctgaaagcacagccgccctgggttgtctggtcaaagactacttccccgaacccgtgacagtgagctggaacagcggcgccctgacaagcggcgtgcacaccttcccagccgtgctgcagagctctggcctgtattctctgagtagcgtggtcaccgttcctagctccaacttcggcacacagacctacacctgtaatgtggaccacaagcctagcaacaccaaagtggataagaccgttgagagaaagtgctgcgtggaatgcccaccatgtccagctccaccagtcgccggcccttctgttttcctgttcccaccaaagcccaaagacaccctgatgatcagccggacccctgaagtgacatgtgtggtggtggatgtgtcccaggaggatcctgaggtgcagtttaattggtacgttgacggagtggaagttcataatgccaagaccaaaccccgcgaggaacagtttaacagcacctaccgggtggtgtccgtgctgacagtgctgcaccaggactggctgaacggcaaagagtacaagtgcaaggtgtctaacaagggcctgcctagcagcatcgagaagaccatctcaaaggccaagggccagcccagagagccccaagtctatacactgcctccttctcaagaagaaatgacaaagaaccaagtgtctctgacctgcctggtgaagggcttctaccccagcgacatcgccgtcgaatgggagagcaacggacagcctgaaaacaactacaagacgacccctccagtgctggacagcgatggcagcttcttcctgtattcacggctgaccgtggacaagagccgatggcaagagggcaacgtgtttagctgcagcgtgctccacgaagccctgcacagccactacacccagaagtccctgagcctgtctctgggaaaataa

SEQ ID NO: 53 - DNA, 642 bp, Artificial Sequence
  Synthetic nucleic acid
gatatccagatgacccagtctccatctagcctgtccgccagcgtgggcgacagagtgaccatcacctgcggcgccagcgagaacatctatggcgctctgaactggtaccagcagaaacctggcaaggcccctaagctgctgatctacggcgccaccaacctggccgatggcgtgcctagtagattcagcggatctggcagcggcacagacttcaccctgaccatcagcagcctgcaacctgaggactttgccacatactactgccagaacgtgctgaatacacctctgacattcggccaaggaaccaaagtggaaatcaagcggaccgtggccgctcctagcgtgttcatcttccctccttccgatgaacaactgaagagcggaaccgcctctgtggtgtgcctgctgaacaacttctaccctagagaggccaaggtgcagtggaaggtcgacaacgccctgcagagcggcaacagccaggagagcgtgacggaacaggacagcaaggacagcacctacagcctgagctccacccttacactgtctaaagccgactacgagaagcacaaggtgtacgcctgtgaagtgacacaccagggcctgagcagccctgtgaccaagtcttttaaccggggcgagtgc

SEQ ID NO: 54 - DNA, 173 bp, Artificial Sequence
  Synthetic nucleic acid
actcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgt

SEQ ID NO: 55 - DNA, 100 bp, Artificial Sequence
  Synthetic nucleic acid
ccaaaatcaacgggactttccaaaatgtcgtaataaccccgccccgttgacgcaaatgggcggtaggcgtgtacggtgggaggtctatataagcagagct

SEQ ID NO: 56 - DNA, 380 bp, Artificial Sequence; identical to SEQ ID NO: 46
  Synthetic nucleic acid

SEQ ID NO: 57 - DNA, 1246 bp, Artificial Sequence; identical to SEQ ID NO: 47
  Synthetic nucleic acid

SEQ ID NO: 58 - DNA, 201 bp, Artificial Sequence
  Synthetic nucleic acid
gaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtgatcatcacttgtga

SEQ ID NO: 59 - DNA, 953 bp, Artificial Sequence; identical to SEQ ID NO: 49
  Synthetic nucleic acid

SEQ ID NO: 60 - DNA, 1766 bp, Artificial Sequence
  Synthetic nucleic acid
gtaaattttatggaatgtgaatcataattcaatttttcaacatgcgttaggagggacatttcaaactcttttttaccctagactttcctaccatcacccagagtatccagccaggaggggaggggctagagacaccagaagtttagcagggaggagggcgtagggattcggggaatgaagggatgggattcagactagggccaggacccagggatggagagaaagagatgagagtggtttgggggcttggtgacttagagaacagagctgcaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggcttaagcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctccagtacgtgattcttgatcccgagctggagccaggggcgggccttgcgctttaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaggatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctccagggggctcaaaatggaggacgcggcgctcgggagagcgggcgggtgagtcacccacacaaaggaaaggggcctttccgtcctcagccgtcgcttcatgtgactccacggagtaccgggcgccgtccaggcacctcgattagttctggagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 61 - DNA, 82 bp, Artificial Sequence
  Synthetic nucleic acid
gtaagggtttaagggatggttggttggtggggtattaatgtttaattacctggagcacctgcctgaaatcactttttttcag

SEQ ID NO: 62 - DNA, 1404 bp, Artificial Sequence
  Synthetic nucleic acid
atgggctggtcctgcatcatcctgttcctggtggccaccgccacaggcgtgcacagccaagtgcagctggttcaaagcggagccgaagtgaaaaagcccggagctagcgtgaaggtgtcctgcaaggccagcggccacatcttcagcaactactggatccagtgggtgcggcaggcccctggccaaggcctggaatggatgggcgagatcctgccaggatctggccacaccgagtacacagagaacttcaaggatagagtgaccatgaccagagatacctccacaagcaccgtgtacatggaactgagcagcctgagaagcgaggacacagctgtgtactactgcgccagatacttttttggctcatcccctaactggtacttcgacgtgtggggccaaggcacccttgtcaccgtcagcagcgccagtactaagggacccagcgtgttcccactggccccatgcagcagaagcacatctgaaagcacagccgccctgggttgtctggtcaaagactacttccccgaacccgtgacagtgagctggaacagcggcgccctgacaagcggcgtgcacaccttcccagccgtgctgcagagctctggcctgtattctctgagtagcgtggtcaccgttcctagctccaacttcggcacacagacctacacctgtaatgtggaccacaagcctagcaacaccaaagtggataagaccgttgagagaaagtgctgcgtggaatgcccaccatgtccagctccaccagtcgccggcccttctgttttcctgttcccaccaaagcccaaagacaccctgatgatcagccggacccctgaagtgacatgtgtggtggtggatgtgtcccaggaggatcctgaggtgcagtttaattggtacgttgacggagtggaagttcataatgccaagaccaaaccccgcgaggaacagtttaacagcacctaccgggtggtgtccgtgctgacagtgctgcaccaggactggctgaacggcaaagagtacaagtgcaaggtgtctaacaagggcctgcctagcagcatcgagaagaccatctcaaaggccaagggccagcccagagagccccaagtctatacactgcctccttctcaagaagaaatgacaaagaaccaagtgtctctgacctgcctggtgaagggcttctaccccagcgacatcgccgtcgaatgggagagcaacggacagcctgaaaacaactacaagacgacccctccagtgctggacagcgatggcagcttcttcctgtattcacggctgaccgtggacaagagccgatggcaagagggcaacgtgtttagctgcagcgtgctccacgaagccctgcacagccactacacccagaagtccctgagcctgtctctgggaaaataa

SEQ ID NO: 63 - DNA, 708 bp, Artificial Sequence
  Synthetic nucleic acid
atggacatgagggtccctgctcagctgctggggctcctgctgctctggctcagcggtgccagatgtgatatccagatgacccagtctccatctagcctgtccgccagcgtgggcgacagagtgaccatcacctgcggcgccagcgagaacatctatggcgctctgaactggtaccagcagaaacctggcaaggcccctaagctgctgatctacggcgccaccaacctggccgatggcgtgcctagtagattcagcggatctggcagcggcacagacttcaccctgaccatcagcagcctgcaacctgaggactttgccacatactactgccagaacgtgctgaatacacctctgacattcggccaaggaaccaaagtggaaatcaagcggaccgtggccgctcctagcgtgttcatcttccctccttccgatgaacaactgaagagcggaaccgcctctgtggtgtgcctgctgaacaacttctaccctagagaggccaaggtgcagtggaaggtcgacaacgccctgcagagcggcaacagccaggagagcgtgacggaacaggacagcaaggacagcacctacagcctgagctccacccttacactgtctaaagccgactacgagaagcacaaggtgtacgcctgtgaagtgacacaccagggcctgagcagccctgtgaccaagtcttttaaccggggcgagtgc

SEQ ID NO: 64 - protein, 448 aa, Artificial Sequence
  Synthetic polypeptide
QVQLVQSGAEVKKPGASVKVSCKASGHIFSNYWIQWVRQAPGQGLEWMGEILPGSGHTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 65 - DNA, 1061 bp, Artificial Sequence; identical to SEQ ID NO: 48
  Synthetic nucleic acid

SEQ ID NO: 66 - DNA, 170 bp, Artificial Sequence
  Synthetic nucleic acid
cgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatca

SEQ ID NO: 67 - DNA, 254 bp, Artificial Sequence
  Synthetic nucleic acid
gggggaggctgctggtgaatattaaccaaggtcaccccagttatcggaggagcaaacaggggctaagtccacctcgagccatggcgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatca

SEQ ID NO: 68 - DNA, 592 bp, Artificial Sequence
  Synthetic nucleic acid
gtaaattttatggaatgtgaatcataattcaatttttcaacatgcgttaggagggacatttcaaactcttttttaccctagactttcctaccatcacccagagtatccagccaggaggggaggggctagagacaccagaagtttagcagggaggagggcgtagggattcggggaatgaagggatgggattcagactagggccaggacccagggatggagagaaagagatgagagtggtttgggggcttggtgacttagagaacagagctgcaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggg

SEQ ID NO: 69 - DNA, 205 bp, Artificial Sequence
  Synthetic nucleic acid
aatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagg

SEQ ID NO: 70 - DNA, 423 bp, Artificial Sequence
  Synthetic nucleic acid
gctctaacccactctgatctcccagggcggcagtaagtcttcagcatcaggcattttggggtgactcagtaaatggtagatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggac

SEQ ID NO: 71 - DNA, 72 bp, Artificial Sequence
  Synthetic nucleic acid
gggggaggctgctggtgaatattaaccaaggtcaccccagttatcggaggagcaaacaggggctaagtccac

SEQ ID NO: 72 - DNA, 913 bp, Artificial Sequence
  Synthetic nucleic acid
tagggaggtcctgcacagaaggggaggagggggcagcagctgtctgaccactgttggtcttgcaacttgtgtccccaggttaatttttaaaaagcagtcaaaagtccaagtggcccttggcagcatttactctctctgtttgctctggttaataatctcaggagcacaaacattcctggaggcaggagaagaaatcaacatcctggacttatcctctgggcctctccccacccccaggattgtaactgaaatgcttcactggtgctccttttgttttaaggcattggatcttcatagctactgatcgtgcccaagcacacagtatctgcagcaaccacttaggcctccaggaatgtggtgaccattgaccctaattcattccccttcatggatcctatgtaaccatcctccaaaaagagctttcgcaaactcaaataaacacaggaaaggaagaccttcttatctttgagagtatatgtttagccctatagctctaacccactctgatctcccagggcggcagtaagtcttcagcatcaggcattttggggtgactcagtaaatggtagatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggac

SEQ ID NO: 73 - DNA, 37 bp, Artificial Sequence
  Synthetic nucleic acid
gtagataagtagcatggcgggttaatcattaactaca

SEQ ID NO: 74 - DNA, 46 bp, Artificial Sequence
  Synthetic nucleic acid
ggaggggtggagtcgtgacgtgaattacgtcatagggttagggagg

SEQ ID NO: 75 - DNA, 182 bp, Artificial Sequence
  Synthetic nucleic acid
gtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 76 - DNA, 191 bp, Artificial Sequence
  Synthetic nucleic acid
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggagg

SEQ ID NO: 77 - protein, 214 aa, Artificial Sequence [sequence omitted]

SEQ ID NO: 78 - protein, 467 aa, Artificial Sequence
  Synthetic polypeptide
MGWSCIILFLVATATGVHSQVQLVQSGAEVKKPGASVKVSCKASGHIFSNYWIQWVRQAPGQGLEWMGEILPGSGHTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 79 - protein, 236 aa, Artificial Sequence
  Synthetic polypeptide
MDMRVPAQLLGLLLLWLSGARCDIQMTQSPSSLSASVGDRVTITCGASENIYGALNWYQQKPGKAPKLLIYGATNLADGVPSRFSGSGSGTDFTLTISSLQPEDFATYYCQNVLNTPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 80 - protein, 19 aa, Artificial Sequence
  Synthetic polypeptide
MGWSCIILFLVATATGVHS

SEQ ID NO: 81 - protein, 22 aa, Artificial Sequence
  Synthetic polypeptide
MDMRVPAQLLGLLLLWLSGARC

SEQ ID NO: 82 - protein, 448 aa, Artificial Sequence
  Synthetic polypeptide
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 83 - DNA, 1344 bp, Artificial Sequence
  Synthetic nucleic acid
caagtgcagctggttcaaagcggagccgaagtgaaaaagcccggagctagcgtgaaggtgtcctgcaaggccagcggctatatcttcagcaactactggatccagtgggtgcggcaggcccctggccaaggcctggaatggatgggcgagatcctgccaggatctggctctaccgagtacacagagaacttcaaggatagagtgaccatgaccagagatacctccacaagcaccgtgtacatggaactgagcagcctgagaagcgaggacacagctgtgtactactgcgccagatacttttttggctcatcccctaactggtacttcgacgtgtggggccaaggcacccttgtcaccgtcagcagcgccagtactaagggacccagcgtgttcccactggccccatgcagcagaagcacatctgaaagcacagccgccctgggttgtctggtcaaagactacttccccgaacccgtgacagtgagctggaacagcggcgccctgacaagcggcgtgcacaccttcccagccgtgctgcagagctctggcctgtattctctgagtagcgtggtcaccgttcctagctccaacttcggcacacagacctacacctgtaatgtggaccacaagcctagcaacaccaaagtggataagaccgttgagagaaagtgctgcgtggaatgcccaccatgtccagctccaccagtcgccggcccttctgttttcctgttcccaccaaagcccaaagacaccctgatgatcagccggacccctgaagtgacatgtgtggtggtggatgtgtcccaggaggatcctgaggtgcagtttaattggtacgttgacggagtggaagttcataatgccaagaccaaaccccgcgaggaacagtttaacagcacctaccgggtggtgtccgtgctgacagtgctgcaccaggactggctgaacggcaaagagtacaagtgcaaggtgtctaacaagggcctgcctagcagcatcgagaagaccatctcaaaggccaagggccagcccagagagccccaagtctatacactgcctccttctcaagaagaaatgacaaagaaccaagtgtctctgacctgcctggtgaagggcttctaccccagcgacatcgccgtcgaatgggagagcaacggacagcctgaaaacaactacaagacgacccctccagtgctggacagcgatggcagcttcttcctgtattcacggctgaccgtggacaagagccgatggcaagagggcaacgtgtttagctgcagcgtgatgcacgaagccctgcacaaccactacacccagaagtccctgagcctgtctctgggaaaa

SEQ ID NO: 84 - DNA, 4181 bp, Artificial Sequence
  Synthetic nucleic acid
gtaaattttatggaatgtgaatcataattcaatttttcaacatgcgttaggagggacatttcaaactcttttttaccctagactttcctaccatcacccagagtatccagccaggaggggaggggctagagacaccagaagtttagcagggaggagggcgtagggattcggggaatgaagggatgggattcagactagggccaggacccagggatggagagaaagagatgagagtggtttgggggcttggtgacttagagaacagagctgcaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggcttaagcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctccagtacgtgattcttgatcccgagctggagccaggggcgggccttgcgctttaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaggatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctccagggggctcaaaatggaggacgcggcgctcgggagagcgggcgggtgagtcacccacacaaaggaaaggggcctttccgtcctcagccgtcgcttcatgtgactccacggagtaccgggcgccgtccaggcacctcgattagttctggagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaactagtgccaccatgggctggtcctgcatcatcctgttcctggtggccaccgccacaggcgtgcacagccaagtgcagctggttcaaagcggagccgaagtgaaaaagcccggagctagcgtgaaggtgtcctgcaaggccagcggccacatcttcagcaactactggatccagtgggtgcggcaggcccctggccaaggcctggaatggatgggcgagatcctgccaggatctggccacaccgagtacacagagaacttcaaggatagagtgaccatgaccagagatacctccacaagcaccgtgtacatggaactgagcagcctgagaagcgaggacacagctgtgtactactgcgccagatacttttttggctcatcccctaactggtacttcgacgtgtggggccaaggcacccttgtcaccgtcagcagcgccagtactaagggacccagcgtgttcccactggccccatgcagcagaagcacatctgaaagcacagccgccctgggttgtctggtcaaagactacttccccgaacccgtgacagtgagctggaacagcggcgccctgacaagcggcgtgcacaccttcccagccgtgctgcagagctctggcctgtattctctgagtagcgtggtcaccgttcctagctccaacttcggcacacagacctacacctgtaatgtggaccacaagcctagcaacaccaaagtggataagaccgttgagagaaagtgctgcgtggaatgcccaccatgtccagctccaccagtcgccggcccttctgttttcctgttcccaccaaagcccaaagacaccctgatgatcagccggacccctgaagtgacatgtgtggtggtggatgtgtcccaggaggatcctgaggtgcagtttaattggtacgttgacggagtggaagttcataatgccaagaccaaaccccgcgaggaacagtttaacagcacctaccgggtggtgtccgtgctgacagtgctgcaccaggactggctgaacggcaaagagtacaagtgcaaggtgtctaacaagggcctgcctagcagcatcgagaagaccatctcaaaggccaagggccagcccagagagccccaagtctatacactgcctccttctcaagaagaaatgacaaagaaccaagtgtctctgacctgcctggtgaagggcttctaccccagcgacatcgccgtcgaatgggagagcaacggacagcctgaaaacaactacaagacgacccctccagtgctggacagcgatggcagcttcttcctgtattcacggctgaccgtggacaagagccgatggcaagagggcaacgtgtttagctgcagcgtgctccacgaagccctgcacagccactacacccagaagtccctgagcctgtctctgggaaaaagaaagagaagaggcagcggagaaggcagaggcagcctgctgacctgcggcgacgtcgaagagaaccccggccctatggacatgagggtccctgctcagctgctggggctcctgctgctctggctcagcggtgccagatgtgatatccagatgacccagtctccatctagcctgtccgccagcgtgggcgacagagtgaccatcacctgcggcgccagcgagaacatctatggcgctctgaactggtaccagcagaaacctggcaaggcccctaagctgctgatctacggcgccaccaacctggccgatggcgtgcctagtagattcagcggatctggcagcggcacagacttcaccctgaccatcagcagcctgcaacctgaggactttgccacatactactgccagaacgtgctgaatacacctctgacattcggccaaggaaccaaagtggaaatcaagcggaccgtggccgctcctagcgtgttcatcttccctccttccgatgaacaactgaagagcggaaccgcctctgtggtgtgcctgctgaacaacttctaccctagagaggccaaggtgcagtggaaggtcgacaacgccctgcagagcggcaacagccaggagagcgtgacggaacaggacagcaaggacagcacctacagcctgagctccacccttacactgtctaaagccgactacgagaagcacaaggtgtacgcctgtgaagtgacacaccagggcctgagcagccctgtgaccaagtcttttaaccggggcgagtgctgaattcgaatcgtacctagggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaa

SEQ ID NO: 85 - DNA, 2956 bp, Artificial Sequence
  Synthetic nucleic acid
ccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcctctaaggtaaatataaaatttttaagtgtataatgtgttaaactactgattctaattgtttctctcttttagattccaacctttggaactgaactagtgccaccatgggctggtcctgcatcatcctgttcctggtggccaccgccacaggcgtgcacagccaagtgcagctggttcaaagcggagccgaagtgaaaaagcccggagctagcgtgaaggtgtcctgcaaggccagcggccacatcttcagcaactactggatccagtgggtgcggcaggcccctggccaaggcctggaatggatgggcgagatcctgccaggatctggccacaccgagtacacagagaacttcaaggatagagtgaccatgaccagagatacctccacaagcaccgtgtacatggaactgagcagcctgagaagcgaggacacagctgtgtactactgcgccagatacttttttggctcatcccctaactggtacttcgacgtgtggggccaaggcacccttgtcaccgtcagcagcgccagtactaagggacccagcgtgttcccactggccccatgcagcagaagcacatctgaaagcacagccgccctgggttgtctggtcaaagactacttccccgaacccgtgacagtgagctggaacagcggcgccctgacaagcggcgtgcacaccttcccagccgtgctgcagagctctggcctgtattctctgagtagcgtggtcaccgttcctagctccaacttcggcacacagacctacacctgtaatgtggaccacaagcctagcaacaccaaagtggataagaccgttgagagaaagtgctgcgtggaatgcccaccatgtccagctccaccagtcgccggcccttctgttttcctgttcccaccaaagcccaaagacaccctgatgatcagccggacccctgaagtgacatgtgtggtggtggatgtgtcccaggaggatcctgaggtgcagtttaattggtacgttgacggagtggaagttcataatgccaagaccaaaccccgcgaggaacagtttaacagcacctaccgggtggtgtccgtgctgacagtgctgcaccaggactggctgaacggcaaagagtacaagtgcaaggtgtctaacaagggcctgcctagcagcatcgagaagaccatctcaaaggccaagggccagcccagagagccccaagtctatacactgcctccttctcaagaagaaatgacaaagaaccaagtgtctctgacctgcctggtgaagggcttctaccccagcgacatcgccgtcgaatgggagagcaacggacagcctgaaaacaactacaagacgacccctccagtgctggacagcgatggcagcttcttcctgtattcacggctgaccgtggacaagagccgatggcaagagggcaacgtgtttagctgcagcgtgctccacgaagccctgcacagccactacacccagaagtccctgagcctgtctctgggaaaaagaaagagaagaggcagcggagaaggcagaggcagcctgctgacctgcggcgacgtcgaagagaaccccggccctatggacatgagggtccctgctcagctgctggggctcctgctgctctggctcagcggtgccagatgtgatatccagatgacccagtctccatctagcctgtccgccagcgtgggcgacagagtgaccatcacctgcggcgccagcgagaacatctatggcgctctgaactggtaccagcagaaacctggcaaggcccctaagctgctgatctacggcgccaccaacctggccgatggcgtgcctagtagattcagcggatctggcagcggcacagacttcaccctgaccatcagcagcctgcaacctgaggactttgccacatactactgccagaacgtgctgaatacacctctgacattcggccaaggaaccaaagtggaaatcaagcggaccgtggccgctcctagcgtgttcatcttccctccttccgatgaacaactgaagagcggaaccgcctctgtggtgtgcctgctgaacaacttctaccctagagaggccaaggtgcagtggaaggtcgacaacgccctgcagagcggcaacagccaggagagcgtgacggaacaggacagcaaggacagcacctacagcctgagctccacccttacactgtctaaagccgactacgagaagcacaaggtgtacgcctgtgaagtgacacaccagggcctgagcagccctgtgaccaagtcttttaaccggggcgagtgctgaattcgaatcgtacctagggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaa

SEQ ID NO: 86 - DNA, 3445 bp, Artificial Sequence
  Synthetic nucleic acid
ccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcctctaaggtaaatataaaatttttaagtgtataatgtgttaaactactgattctaattgtttctctcttttagattccaacctttggaactgaactagtgccaccatgggctggtcctgcatcatcctgttcctggtggccaccgccacaggcgtgcacagccaagtgcagctggttcaaagcggagccgaagtgaaaaagcccggagctagcgtgaaggtgtcctgcaaggccagcggccacatcttcagcaactactggatccagtgggtgcggcaggcccctggccaaggcctggaatggatgggcgagatcctgccaggatctggccacaccgagtacacagagaacttcaaggatagagtgaccatgaccagagatacctccacaagcaccgtgtacatggaactgagcagcctgagaagcgaggacacagctgtgtactactgcgccagatacttttttggctcatcccctaactggtacttcgacgtgtggggccaaggcacccttgtcaccgtcagcagcgccagtactaagggacccagcgtgttcccactggccccatgcagcagaagcacatctgaaagcacagccgccctgggttgtctggtcaaagactacttccccgaacccgtgacagtgagctggaacagcggcgccctgacaagcggcgtgcacaccttcccagccgtgctgcagagctctggcctgtattctctgagtagcgtggtcaccgttcctagctccaacttcggcacacagacctacacctgtaatgtggaccacaagcctagcaacaccaaagtggataagaccgttgagagaaagtgctgcgtggaatgcccaccatgtccagctccaccagtcgccggcccttctgttttcctgttcccaccaaagcccaaagacaccctgatgatcagccggacccctgaagtgacatgtgtggtggtggatgtgtcccaggaggatcctgaggtgcagtttaattggtacgttgacggagtggaagttcataatgccaagaccaaaccccgcgaggaacagtttaacagcacctaccgggtggtgtccgtgctgacagtgctgcaccaggactggctgaacggcaaagagtacaagtgcaaggtgtctaacaagggcctgcctagcagcatcgagaagaccatctcaaaggccaagggccagcccagagagccccaagtctatacactgcctccttctcaagaagaaatgacaaagaaccaagtgtctctgacctgcctggtgaagggcttctaccccagcgacatcgccgtcgaatgggagagcaacggacagcctgaaaacaactacaagacgacccctccagtgctggacagcgatggcagcttcttcctgtattcacggctgaccgtggacaagagccgatggcaagagggcaacgtgtttagctgcagcgtgctccacgaagccctgcacagccactacacccagaagtccctgagcctgtctctgggaaaataaaacgttcctcgaggctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaaggtccggagggggaggctgctggtgaatattaaccaaggtcaccccagttatcggaggagcaaacaggggctaagtccacctcgagccatggcgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagcgccggcaagaggtaagggtttaagggatggttggttggtggggtattaatgtttaattacctggagcacctgcctgaaatcactttttttcaggttggaccggtgccaccatggacatgagggtccctgctcagctgctggggctcctgctgctctggctcagcggtgccagatgtgatatccagatgacccagtctccatctagcctgtccgccagcgtgggcgacagagtgaccatcacctgcggcgccagcgagaacatctatggcgctctgaactggtaccagcagaaacctggcaaggcccctaagctgctgatctacggcgccaccaacctggccgatggcgtgcctagtagattcagcggatctggcagcggcacagacttcaccctgaccatcagcagcctgcaacctgaggactttgccacatactactgccagaacgtgctgaatacacctctgacattcggccaaggaaccaaagtggaaatcaagcggaccgtggccgctcctagcgtgttcatcttccctccttccgatgaacaactgaagagcggaaccgcctctgtggtgtgcctgctgaacaacttctaccctagagaggccaaggtgcagtggaaggtcgacaacgccctgcagagcggcaacagccaggagagcgtgacggaacaggacagcaaggacagcacctacagcctgagctccacccttacactgtctaaagccgactacgagaagcacaaggtgtacgcctgtgaagtgacacaccagggcctgagcagccctgtgaccaagtcttttaaccggggcgagtgctgaattcgaatcgtacctagggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaa

SEQ ID NO: 87 - DNA, 4075 bp, Artificial Sequence
  Synthetic nucleic acid
ccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagcctcgaggaacgttttattttcccagagacaggctcagggacttctgggtgtagtggctgtgcagggcttcgtggagcacgctgcagctaaacacgttgccctcttgccatcggctcttgtccacggtcagccgtgaatacaggaagaagctgccatcgctgtccagcactggaggggtcgtcttgtagttgttttcaggctgtccgttgctctcccattcgacggcgatgtcgctggggtagaagcccttcaccaggcaggtcagagacacttggttctttgtcatttcttcttgagaaggaggcagtgtatagacttggggctctctgggctggcccttggcctttgagatggtcttctcgatgctgctaggcaggcccttgttagacaccttgcacttgtactctttgccgttcagccagtcctggtgcagcactgtcagcacggacaccacccggtaggtgctgttaaactgttcctcgcggggtttggtcttggcattatgaacttccactccgtcaacgtaccaattaaactgcacctcaggatcctcctgggacacatccaccaccacacatgtcacttcaggggtccggctgatcatcagggtgtctttgggctttggtgggaacaggaaaacagaagggccggcgactggtggagctggacatggtgggcattccacgcagcactttctctcaacggtcttatccactttggtgttgctaggcttgtggtccacattacaggtgtaggtctgtgtgccgaagttggagctaggaacggtgaccacgctactcagagaatacaggccagagctctgcagcacggctgggaaggtgtgcacgccgcttgtcagggcgccgctgttccagctcactgtcacgggttcggggaagtagtctttgaccagacaacccagggcggctgtgctttcagatgtgcttctgctgcatggggccagtgggaacacgctgggtcccttagtactggcgctgctgacggtgacaagggtgccttggccccacacgtcgaagtaccagttaggggatgagccaaaaaagtatctggcgcagtagtacacagctgtgtcctcgcttctcaggctgctcagttccatgtacacggtgcttgtggaggtatctctggtcatggtcactctatccttgaagttctctgtgtactcggtgtggccagatcctggcaggatctcgcccatccattccaggccttggccaggggcctgccgcacccactggatccagtagttgctgaagatgtggccgctggccttgcaggacaccttcacgctagctccgggctttttcacttcggctccgctttgaaccagctgcacttggctgtgcacgcctgtggcggtggccaccaggaacaggatgatgcaggaccagcccatggtggcactagttcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattcccctctcacactacctaaaccacgccaggacaacctctgctcctctccaccgaaattccaaggggtcgagtggatgttggaggtggcatgggcccagagaggtctctgacctctgccccagctccaaggtcagcaggcagggagggctgtgtgtttgctgtttgctgcttgcaatgtttgcccattttagggccgcggcacgtgcttaaggcccccttttgcatccagtttattcctacatttgtcacactgttaacagcccaccccttccaatgagaccagtggtatcagtgagttgtggagatcaggaaaagggctcaagagaaaggcagtcaaagccctttttctgtccctgtcccagctgctttaataagatctccataagagaagagggacagctatgactgggagtagtcaggagaggaggaaaaatctggctagtaaaacatgtaaggaaaattttagggatgttaaagaaaaaaataacacaaaacaaaatataaaaaaaatctaacctcaagtcaaggcttttctatggaataaggaatggacagcagggggctgtttcatatactgatgacctctttatagccaacctttgttcatggcagccagcatatgggcatatgttgccaaactctaaaccaaatactcattctgatgttttaaatgatttgccctcccatatgtccttccgagtgagagacacaaaaaattccaacacactattgcaatgaaaataaatttcctttattagccagaagtcagatgctcaaggggcttcatgatgtccccataatttttggcagagggaaaaagatctccggagggggaggctgctggtgaatattaaccaaggtcaccccagttatcggaggagcaaacaggggctaagtccacctcgagccatggcgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagcgccggcaagaggtaagggtttaagggatggttggttggtggggtattaatgtttaattacctggagcacctgcctgaaatcactttttttcaggttggaccggtgccaccatggacatgagggtccctgctcagctgctggggctcctgctgctctggctcagcggtgccagatgtgatatccagatgacccagtctccatctagcctgtccgccagcgtgggcgacagagtgaccatcacctgcggcgccagcgagaacatctatggcgctctgaactggtaccagcagaaacctggcaaggcccctaagctgctgatctacggcgccaccaacctggccgatggcgtgcctagtagattcagcggatctggcagcggcacagacttcaccctgaccatcagcagcctgcaacctgaggactttgccacatactactgccagaacgtgctgaatacacctctgacattcggccaaggaaccaaagtggaaatcaagcggaccgtggccgctcctagcgtgttcatcttccctccttccgatgaacaactgaagagcggaaccgcctctgtggtgtgcctgctgaacaacttctaccctagagaggccaaggtgcagtggaaggtcgacaacgccctgcagagcggcaacagccaggagagcgtgacggaacaggacagcaaggacagcacctacagcctgagctccacccttacactgtctaaagccgactacgagaagcacaaggtgtacgcctgtgaagtgacacaccagggcctgagcagccctgtgaccaagtcttttaaccggggcgagtgctgaattcgaatcgtacctagggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaa

SEQ ID NO: 88 - DNA, 4588 bp, Artificial Sequence
  Synthetic nucleic acid
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgcatatgcggccgcgtaaattttatggaatgtgaatcataattcaatttttcaacatgcgttaggagggacatttcaaactcttttttaccctagactttcctaccatcacccagagtatccagccaggaggggaggggctagagacaccagaagtttagcagggaggagggcgtagggattcggggaatgaagggatgggattcagactagggccaggacccagggatggagagaaagagatgagagtggtttgggggcttggtgacttagagaacagagctgcaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggcttaagcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctccagtacgtgattcttgatcccgagctggagccaggggcgggccttgcgctttaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaggatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctccagggggctcaaaatggaggacgcggcgctcgggagagcgggcgggtgagtcacccacacaaaggaaaggggcctttccgtcctcagccgtcgcttcatgtgactccacggagtaccgggcgccgtccaggcacctcgattagttctggagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaactagtgccaccatgggctggtcctgcatcatcctgttcctggtggccaccgccacaggcgtgcacagccaagtgcagctggttcaaagcggagccgaagtgaaaaagcccggagctagcgtgaaggtgtcctgcaaggccagcggccacatcttcagcaactactggatccagtgggtgcggcaggcccctggccaaggcctggaatggatgggcgagatcctgccaggatctggccacaccgagtacacagagaacttcaaggatagagtgaccatgaccagagatacctccacaagcaccgtgtacatggaactgagcagcctgagaagcgaggacacagctgtgtactactgcgccagatacttttttggctcatcccctaactggtacttcgacgtgtggggccaaggcacccttgtcaccgtcagcagcgccagtactaagggacccagcgtgttcccactggccccatgcagcagaagcacatctgaaagcacagccgccctgggttgtctggtcaaagactacttccccgaacccgtgacagtgagctggaacagcggcgccctgacaagcggcgtgcacaccttcccagccgtgctgcagagctctggcctgtattctctgagtagcgtggtcaccgttcctagctccaacttcggcacacagacctacacctgtaatgtggaccacaagcctagcaacaccaaagtggataagaccgttgagagaaagtgctgcgtggaatgcccaccatgtccagctccaccagtcgccggcccttctgttttcctgttcccaccaaagcccaaagacaccctgatgatcagccggacccctgaagtgacatgtgtggtggtggatgtgtcccaggaggatcctgaggtgcagtttaattggtacgttgacggagtggaagttcataatgccaagaccaaaccccgcgaggaacagtttaacagcacctaccgggtggtgtccgtgctgacagtgctgcaccaggactggctgaacggcaaagagtacaagtgcaaggtgtctaacaagggcctgcctagcagcatcgagaagaccatctcaaaggccaagggccagcccagagagccccaagtctatacactgcctccttctcaagaagaaatgacaaagaaccaagtgtctctgacctgcctggtgaagggcttctaccccagcgacatcgccgtcgaatgggagagcaacggacagcctgaaaacaactacaagacgacccctccagtgctggacagcgatggcagcttcttcctgtattcacggctgaccgtggacaagagccgatggcaagagggcaacgtgtttagctgcagcgtgctccacgaagccctgcacagccactacacccagaagtccctgagcctgtctctgggaaaaagaaagagaagaggcagcggagaaggcagaggcagcctgctgacctgcggcgacgtcgaagagaaccccggccctatggacatgagggtccctgctcagctgctggggctcctgctgctctggctcagcggtgccagatgtgatatccagatgacccagtctccatctagcctgtccgccagcgtgggcgacagagtgaccatcacctgcggcgccagcgagaacatctatggcgctctgaactggtaccagcagaaacctggcaaggcccctaagctgctgatctacggcgccaccaacctggccgatggcgtgcctagtagattcagcggatctggcagcggcacagacttcaccctgaccatcagcagcctgcaacctgaggactttgccacatactactgccagaacgtgctgaatacacctctgacattcggccaaggaaccaaagtggaaatcaagcggaccgtggccgctcctagcgtgttcatcttccctccttccgatgaacaactgaagagcggaaccgcctctgtggtgtgcctgctgaacaacttctaccctagagaggccaaggtgcagtggaaggtcgacaacgccctgcagagcggcaacagccaggagagcgtgacggaacaggacagcaaggacagcacctacagcctgagctccacccttacactgtctaaagccgactacgagaagcacaaggtgtacgcctgtgaagtgacacaccagggcctgagcagccctgtgaccaagtcttttaaccggggcgagtgctgaattcgaatcgtacctagggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaagcttgtttaaacgtacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 89 - DNA, 3375 bp, Artificial Sequence
  Synthetic nucleic acid
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgcatatgcggccgcacgtgccgcggccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcctctaaggtaaatataaaatttttaagtgtataatgtgttaaactactgattctaattgtttctctcttttagattccaacctttggaactgaactagtgccaccatgggctggtcctgcatcatcctgttcctggtggccaccgccacaggcgtgcacagccaagtgcagctggttcaaagcggagccgaagtgaaaaagcccggagctagcgtgaaggtgtcctgcaaggccagcggccacatcttcagcaactactggatccagtgggtgcggcaggcccctggccaaggcctggaatggatgggcgagatcctgccaggatctggccacaccgagtacacagagaacttcaaggatagagtgaccatgaccagagatacctccacaagcaccgtgtacatggaactgagcagcctgagaagcgaggacacagctgtgtactactgcgccagatacttttttggctcatcccctaactggtacttcgacgtgtggggccaaggcacccttgtcaccgtcagcagcgccagtactaagggacccagcgtgttcccactggccccatgcagcagaagcacatctgaaagcacagccgccctgggttgtctggtcaaagactacttccccgaacccgtgacagtgagctggaacagcggcgccctgacaagcggcgtgcacaccttcccagccgtgctgcagagctctggcctgtattctctgagtagcgtggtcaccgttcctagctccaacttcggcacacagacctacacctgtaatgtggaccacaagcctagcaacaccaaagtggataagaccgttgagagaaagtgctgcgtggaatgcccaccatgtccagctccaccagtcgccggcccttctgttttcctgttcccaccaaagcccaaagacaccctgatgatcagccggacccctgaagtgacatgtgtggtggtggatgtgtcccaggaggatcctgaggtgcagtttaattggtacgttgacggagtggaagttcataatgccaagaccaaaccccgcgaggaacagtttaacagcacctaccgggtggtgtccgtgctgacagtgctgcaccaggactggctgaacggcaaagagtacaagtgcaaggtgtctaacaagggcctgcctagcagcatcgagaagaccatctcaaaggccaagggccagcccagagagccccaagtctatacactgcctccttctcaagaagaaatgacaaagaaccaagtgtctctgacctgcctggtgaagggcttctaccccagcgacatcgccgtcgaatgggagagcaacggacagcctgaaaacaactacaagacgacccctccagtgctggacagcgatggcagcttcttcctgtattcacggctgaccgtggacaagagccgatggcaagagggcaacgtgtttagctgcagcgtgctccacgaagccctgcacagccactacacccagaagtccctgagcctgtctctgggaaaaagaaagagaagaggcagcggagaaggcagaggcagcctgctgacctgcggcgacgtcgaagagaaccccggccctatggacatgagggtccctgctcagctgctggggctcctgctgctctggctcagcggtgccagatgtgatatccagatgacccagtctccatctagcctgtccgccagcgtgggcgacagagtgaccatcacctgcggcgccagcgagaacatctatggcgctctgaactggtaccagcagaaacctggcaaggcccctaagctgctgatctacggcgccaccaacctggccgatggcgtgcctagtagattcagcggatctggcagcggcacagacttcaccctgaccatcagcagcctgcaacctgaggactttgccacatactactgccagaacgtgctgaatacacctctgacattcggccaaggaaccaaagtggaaatcaagcggaccgtggccgctcctagcgtgttcatcttccctccttccgatgaacaactgaagagcggaaccgcctctgtggtgtgcctgctgaacaacttctaccctagagaggccaaggtgcagtggaaggtcgacaacgccctgcagagcggcaacagccaggagagcgtgacggaacaggacagcaaggacagcacctacagcctgagctccacccttacactgtctaaagccgactacgagaagcacaaggtgtacgcctgtgaagtgacacaccagggcctgagcagccctgtgaccaagtcttttaaccggggcgagtgctgaattcgaatcgtacctagggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaacctgcaggtctagatacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 90 - DNA, 3863 bp, Artificial Sequence
  Synthetic nucleic acid
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgcatatgcggccgcacgtgccgcggccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcctctaaggtaaatataaaatttttaagtgtataatgtgttaaactactgattctaattgtttctctcttttagattccaacctttggaactgaactagtgccaccatgggctggtcctgcatcatcctgttcctggtggccaccgccacaggcgtgcacagccaagtgcagctggttcaaagcggagccgaagtgaaaaagcccggagctagcgtgaaggtgtcctgcaaggccagcggccacatcttcagcaactactggatccagtgggtgcggcaggcccctggccaaggcctggaatggatgggcgagatcctgccaggatctggccacaccgagtacacagagaacttcaaggatagagtgaccatgaccagagatacctccacaagcaccgtgtacatggaactgagcagcctgagaagcgaggacacagctgtgtactactgcgccagatacttttttggctcatcccctaactggtacttcgacgtgtggggccaaggcacccttgtcaccgtcagcagcgccagtactaagggacccagcgtgttcccactggccccatgcagcagaagcacatctgaaagcacagccgccctgggttgtctggtcaaagactacttccccgaacccgtgacagtgagctggaacagcggcgccctgacaagcggcgtgcacaccttcccagccgtgctgcagagctctggcctgtattctctgagtagcgtggtcaccgttcctagctccaacttcggcacacagacctacacctgtaatgtggaccacaagcctagcaacaccaaagtggataagaccgttgagagaaagtgctgcgtggaatgcccaccatgtccagctccaccagtcgccggcccttctgttttcctgttcccaccaaagcccaaagacaccctgatgatcagccggacccctgaagtgacatgtgtggtggtggatgtgtcccaggaggatcctgaggtgcagtttaattggtacgttgacggagtggaagttcataatgccaagaccaaaccccgcgaggaacagtttaacagcacctaccgggtggtgtccgtgctgacagtgctgcaccaggactggctgaacggcaaagagtacaagtgcaaggtgtctaacaagggcctgcctagcagcatcgagaagaccatctcaaaggccaagggccagcccagagagccccaagtctatacactgcctccttctcaagaagaaatgacaaagaaccaagtgtctctgacctgcctggtgaagggcttctaccccagcgacatcgccgtcgaatgggagagcaacggacagcctgaaaacaactacaagacgacccctccagtgctggacagcgatggcagcttcttcctgtattcacggctgaccgtggacaagagccgatggcaagagggcaacgtgtttagctgcagcgtgctccacgaagccctgcacagccactacacccagaagtccctgagcctgtctctgggaaaataaaacgttcctcgaggctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaaggtccggagggggaggctgctggtgaatattaaccaaggtcaccccagttatcggaggagcaaacaggggctaagtccacctcgagccatggcgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagcgccggcaagaggtaagggtttaagggatggttggttggtggggtattaatgtttaattacctggagcacctgcctgaaatcactttttttcaggttggaccggtgccaccatggacatgagggtccctgctcagctgctggggctcctgctgctctggctcagcggtgccagatgtgatatccagatgacccagtctccatctagcctgtccgccagcgtgggcgacagagtgaccatcacctgcggcgccagcgagaacatctatggcgctctgaactggtaccagcagaaacctggcaaggcccctaagctgctgatctacggcgccaccaacctggccgatggcgtgcctagtagattcagcggatctggcagcggcacagacttcaccctgaccatcagcagcctgcaacctgaggactttgccacatactactgccagaacgtgctgaatacacctctgacattcggccaaggaaccaaagtggaaatcaagcggaccgtggccgctcctagcgtgttcatcttccctccttccgatgaacaactgaagagcggaaccgcctctgtggtgtgcctgctgaacaacttctaccctagagaggccaaggtgcagtggaaggtcgacaacgccctgcagagcggcaacagccaggagagcgtgacggaacaggacagcaaggacagcacctacagcctgagctccacccttacactgtctaaagccgactacgagaagcacaaggtgtacgcctgtgaagtgacacaccagggcctgagcagccctgtgaccaagtcttttaaccggggcgagtgctgaattcgaatcgtacctagggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaagcttgtttaaacgtacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 91 - DNA, 4573 bp, Artificial Sequence
  Synthetic nucleic acid
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgcatatgcggccgcgtcatagggttagggaggtcctgcacacgtgacgcgtccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagcctcgaggaacgttttattttcccagagacaggctcagggacttctgggtgtagtggctgtgcagggcttcgtggagcacgctgcagctaaacacgttgccctcttgccatcggctcttgtccacggtcagccgtgaatacaggaagaagctgccatcgctgtccagcactggaggggtcgtcttgtagttgttttcaggctgtccgttgctctcccattcgacggcgatgtcgctggggtagaagcccttcaccaggcaggtcagagacacttggttctttgtcatttcttcttgagaaggaggcagtgtatagacttggggctctctgggctggcccttggcctttgagatggtcttctcgatgctgctaggcaggcccttgttagacaccttgcacttgtactctttgccgttcagccagtcctggtgcagcactgtcagcacggacaccacccggtaggtgctgttaaactgttcctcgcggggtttggtcttggcattatgaacttccactccgtcaacgtaccaattaaactgcacctcaggatcctcctgggacacatccaccaccacacatgtcacttcaggggtccggctgatcatcagggtgtctttgggctttggtgggaacaggaaaacagaagggccggcgactggtggagctggacatggtgggcattccacgcagcactttctctcaacggtcttatccactttggtgttgctaggcttgtggtccacattacaggtgtaggtctgtgtgccgaagttggagctaggaacggtgaccacgctactcagagaatacaggccagagctctgcagcacggctgggaaggtgtgcacgccgcttgtcagggcgccgctgttccagctcactgtcacgggttcggggaagtagtctttgaccagacaacccagggcggctgtgctttcagatgtgcttctgctgcatggggccagtgggaacacgctgggtcccttagtactggcgctgctgacggtgacaagggtgccttggccccacacgtcgaagtaccagttaggggatgagccaaaaaagtatctggcgcagtagtacacagctgtgtcctcgcttctcaggctgctcagttccatgtacacggtgcttgtggaggtatctctggtcatggtcactctatccttgaagttctctgtgtactcggtgtggccagatcctggcaggatctcgcccatccattccaggccttggccaggggcctgccgcacccactggatccagtagttgctgaagatgtggccgctggccttgcaggacaccttcacgctagctccgggctttttcacttcggctccgctttgaaccagctgcacttggctgtgcacgcctgtggcggtggccaccaggaacaggatgatgcaggaccagcccatggtggcactagttcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattcccctctcacactacctaaaccacgccaggacaacctctgctcctctccaccgaaattccaaggggtcgagtggatgttggaggtggcatgggcccagagaggtctctgacctctgccccagctccaaggtcagcaggcagggagggctgtgtgtttgctgtttgctgcttgcaatgtttgcccattttagggccgcggcacgtgcttaaggcccccttttgcatccagtttattcctacatttgtcacactgttaacagcccaccccttccaatgagaccagtggtatcagtgagttgtggagatcaggaaaagggctcaagagaaaggcagtcaaagccctttttctgtccctgtcccagctgctttaataagatctccataagagaagagggacagctatgactgggagtagtcaggagaggaggaaaaatctggctagtaaaacatgtaaggaaaattttagggatgttaaagaaaaaaataacacaaaacaaaatataaaaaaaatctaacctcaagtcaaggcttttctatggaataaggaatggacagcagggggctgtttcatatactgatgacctctttatagccaacctttgttcatggcagccagcatatgggcatatgttgccaaactctaaaccaaatactcattctgatgttttaaatgatttgccctcccatatgtccttccgagtgagagacacaaaaaattccaacacactattgcaatgaaaataaatttcctttattagccagaagtcagatgctcaaggggcttcatgatgtccccataatttttggcagagggaaaaagatctccggagggggaggctgctggtgaatattaaccaaggtcaccccagttatcggaggagcaaacaggggctaagtccacctcgagccatggcgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagcgccggcaagaggtaagggtttaagggatggttggttggtggggtattaatgtttaattacctggagcacctgcctgaaatcactttttttcaggttggaccggtgccaccatggacatgagggtccctgctcagctgctggggctcctgctgctctggctcagcggtgccagatgtgatatccagatgacccagtctccatctagcctgtccgccagcgtgggcgacagagtgaccatcacctgcggcgccagcgagaacatctatggcgctctgaactggtaccagcagaaacctggcaaggcccctaagctgctgatctacggcgccaccaacctggccgatggcgtgcctagtagattcagcggatctggcagcggcacagacttcaccctgaccatcagcagcctgcaacctgaggactttgccacatactactgccagaacgtgctgaatacacctctgacattcggccaaggaaccaaagtggaaatcaagcggaccgtggccgctcctagcgtgttcatcttccctccttccgatgaacaactgaagagcggaaccgcctctgtggtgtgcctgctgaacaacttctaccctagagaggccaaggtgcagtggaaggtcgacaacgccctgcagagcggcaacagccaggagagcgtgacggaacaggacagcaaggacagcacctacagcctgagctccacccttacactgtctaaagccgactacgagaagcacaaggtgtacgcctgtgaagtgacacaccagggcctgagcagccctgtgaccaagtcttttaaccggggcgagtgctgaattcgaatcgtacctagggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaagcttgtttaaacgtacgtagataagtagcatggcgggttaatcattaactacacctgcaggtctagatacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 92 - DNA, 2142 bp, Artificial Sequence
  Synthetic nucleic acid
ccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcctctaaggtaaatataaaatttttaagtgtataatgtgttaaactactgattctaattgtttctctcttttagattccaacctttggaactgaactagtgccaccatgggctggtcctgcatcatcctgttcctggtggccaccgccacaggcgtgcacagccaagtgcagctggttcaaagcggagccgaagtgaaaaagcccggagctagcgtgaaggtgtcctgcaaggccagcggccacatcttcagcaactactggatccagtgggtgcggcaggcccctggccaaggcctggaatggatgggcgagatcctgccaggatctggccacaccgagtacacagagaacttcaaggatagagtgaccatgaccagagatacctccacaagcaccgtgtacatggaactgagcagcctgagaagcgaggacacagctgtgtactactgcgccagatacttttttggctcatcccctaactggtacttcgacgtgtggggccaaggcacccttgtcaccgtcagcagcgccagtactaagggacccagcgtgttcccactggccccatgcagcagaagcacatctgaaagcacagccgccctgggttgtctggtcaaagactacttccccgaacccgtgacagtgagctggaacagcggcgccctgacaagcggcgtgcacaccttcccagccgtgctgcagagctctggcctgtattctctgagtagcgtggtcaccgttcctagctccaacttcggcacacagacctacacctgtaatgtggaccacaagcctagcaacaccaaagtggataagaccgttgagagaaagtgctgcgtggaatgcccaccatgtccagctccaccagtcgccggcccttctgttttcctgttcccaccaaagcccaaagacaccctgatgatcagccggacccctgaagtgacatgtgtggtggtggatgtgtcccaggaggatcctgaggtgcagtttaattggtacgttgacggagtggaagttcataatgccaagaccaaaccccgcgaggaacagtttaacagcacctaccgggtggtgtccgtgctgacagtgctgcaccaggactggctgaacggcaaagagtacaagtgcaaggtgtctaacaagggcctgcctagcagcatcgagaagaccatctcaaaggccaagggccagcccagagagccccaagtctatacactgcctccttctcaagaagaaatgacaaagaaccaagtgtctctgacctgcctggtgaagggcttctaccccagcgacatcgccgtcgaatgggagagcaacggacagcctgaaaacaactacaagacgacccctccagtgctggacagcgatggcagcttcttcctgtattcacggctgaccgtggacaagagccgatggcaagagggcaacgtgtttagctgcagcgtgctccacgaagccctgcacagccactacacccagaagtccctgagcctgtctctgggaaaataaaacgttcctcgaggctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagg

SEQ ID NO: 93 - DNA, 1297 bp, Artificial Sequence
  Synthetic nucleic acid
gggggaggctgctggtgaatattaaccaaggtcaccccagttatcggaggagcaaacaggggctaagtccacctcgagccatggcgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagcgccggcaagaggtaagggtttaagggatggttggttggtggggtattaatgtttaattacctggagcacctgcctgaaatcactttttttcaggttggaccggtgccaccatggacatgagggtccctgctcagctgctggggctcctgctgctctggctcagcggtgccagatgtgatatccagatgacccagtctccatctagcctgtccgccagcgtgggcgacagagtgaccatcacctgcggcgccagcgagaacatctatggcgctctgaactggtaccagcagaaacctggcaaggcccctaagctgctgatctacggcgccaccaacctggccgatggcgtgcctagtagattcagcggatctggcagcggcacagacttcaccctgaccatcagcagcctgcaacctgaggactttgccacatactactgccagaacgtgctgaatacacctctgacattcggccaaggaaccaaagtggaaatcaagcggaccgtggccgctcctagcgtgttcatcttccctccttccgatgaacaactgaagagcggaaccgcctctgtggtgtgcctgctgaacaacttctaccctagagaggccaaggtgcagtggaaggtcgacaacgccctgcagagcggcaacagccaggagagcgtgacggaacaggacagcaaggacagcacctacagcctgagctccacccttacactgtctaaagccgactacgagaagcacaaggtgtacgcctgtgaagtgacacaccagggcctgagcagccctgtgaccaagtcttttaaccggggcgagtgctgaattcgaatcgtacctagggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaa

SEQ ID NO: 94 - DNA, 1344 bp, Artificial Sequence; identical to SEQ ID NO: 83
  Synthetic nucleic acid

SEQ ID NO: 95 - DNA, 1344 bp, Artificial Sequence
  Synthetic nucleic acid
caagtacagcttgtacaaagtggcgctgaagtaaaaaagccaggggcttcagtgaaagtgtcctgcaaggcctcgggctacatcttctccaactactggatccagtgggtgcgccaggccccgggccaagggctggagtggatgggagagattttacctggcagtggctccacagagtacacagagaacttcaaggacagagtcaccatgacccgggacaccagcacatcaactgtctacatggagctgagctccctccgatcagaagacacagctgtctactactgtgcccgctacttctttggcagcagccccaattggtattttgatgtgtggggccaaggcaccttggtcaccgtctcctcagcatcaacaaaaggtccttctgtcttccccctggccccctgcagccgcagcacatcagaatccacagcagccctgggctgcctggtaaaagactacttcccagaacctgtcactgtctcctggaacagtggagccctgacatctggtgtccacaccttccctgctgtcctccagagctctggcctctacagcctctcctcagtggtcacagttccatcctccaactttggcacacagacctacacctgcaatgtggaccacaagccttccaacaccaaggtggacaagactgtagagaggaagtgctgtgtggaatgcccaccctgcccagctccacctgttgctgggccttctgtcttcctcttccctccaaagcccaaagacaccctcatgatcagccgcaccccagaagtgacctgtgtggtggtggatgtctcccaggaagaccctgaggtgcagttcaactggtatgtagatggtgttgaagttcataatgccaagaccaagccccgggaggagcagttcaacagcacctacagagtggtgtctgtgctgacagtgctgcaccaggactggctgaatggaaaagaatacaagtgcaaagtttccaacaagggcctgccttcctccattgagaagaccatctccaaagccaagggacagccccgggagccccaagtctacacacttcctccttctcaagaagaaatgacaaagaaccaagtgtctctgacctgcctggtgaagggcttctacccaagtgacattgctgtagaatgggagagcaatggacagcctgaaaacaactacaaaacgaccccacctgtgctggactctgatggcagcttcttcttgtattcacgcctgactgtggacaagagccgctggcaagaaggaaatgtattttcctgctctgtgatgcatgaggccctgcacaatcactacacccagaagtcactcagcctctctctgggaaaa

SEQ ID NO: 96 - DNA, 57 bp, Artificial Sequence
  Synthetic nucleic acid
atgggctggagctgcatcatcctcttcctggtggccacggccacaggtgtccacagc

SEQ ID NO: 97 - DNA, 1401 bp, Artificial Sequence
  Synthetic nucleic acid
atgggctggagctgcatcatcctcttcctggtggccacggccacaggtgtccacagccaagtacagcttgtacaaagtggcgctgaagtaaaaaagccaggggcttcagtgaaagtgtcctgcaaggcctcgggctacatcttctccaactactggatccagtgggtgcgccaggccccgggccaagggctggagtggatgggagagattttacctggcagtggctccacagagtacacagagaacttcaaggacagagtcaccatgacccgggacaccagcacatcaactgtctacatggagctgagctccctccgatcagaagacacagctgtctactactgtgcccgctacttctttggcagcagccccaattggtattttgatgtgtggggccaaggcaccttggtcaccgtctcctcagcatcaacaaaaggtccttctgtcttccccctggccccctgcagccgcagcacatcagaatccacagcagccctgggctgcctggtaaaagactacttcccagaacctgtcactgtctcctggaacagtggagccctgacatctggtgtccacaccttccctgctgtcctccagagctctggcctctacagcctctcctcagtggtcacagttccatcctccaactttggcacacagacctacacctgcaatgtggaccacaagccttccaacaccaaggtggacaagactgtagagaggaagtgctgtgtggaatgcccaccctgcccagctccacctgttgctgggccttctgtcttcctcttccctccaaagcccaaagacaccctcatgatcagccgcaccccagaagtgacctgtgtggtggtggatgtctcccaggaagaccctgaggtgcagttcaactggtatgtagatggtgttgaagttcataatgccaagaccaagccccgggaggagcagttcaacagcacctacagagtggtgtctgtgctgacagtgctgcaccaggactggctgaatggaaaagaatacaagtgcaaagtttccaacaagggcctgccttcctccattgagaagaccatctccaaagccaagggacagccccgggagccccaagtctacacacttcctccttctcaagaagaaatgacaaagaaccaagtgtctctgacctgcctggtgaagggcttctacccaagtgacattgctgtagaatgggagagcaatggacagcctgaaaacaactacaaaacgaccccacctgtgctggactctgatggcagcttcttcttgtattcacgcctgactgtggacaagagccgctggcaagaaggaaatgtattttcctgctctgtgatgcatgaggccctgcacaatcactacacccagaagtcactcagcctctctctgggaaaa

SEQ ID NO: 98 - DNA, 642 bp, Artificial Sequence
  Synthetic nucleic acid
gacatccagatgacacagagcccttccagcctctctgcctcggtgggggacagagtcaccatcacctgtggggcctcggagaacatctatggtgctctgaactggtaccagcagaaacctgggaaggcccccaagctgctcatctatggggccaccaacctggctgatggtgtgccctcgcgcttctctggatctggaagtggcacagacttcaccctcaccatctccagcctgcaacctgaggactttgccacctactactgccagaatgtgctgaacacacccctcacttttggacaagggaccaaagtagaaatcaagaggacagtggcggcgcccagtgtcttcatcttccctccaagcgatgaacaactaaagtctggaacagcctcggtggtctgcctgctgaacaacttctacccaagagaggccaaggtgcagtggaaagttgataatgccctgcagagtggaaacagccaggagagtgtcactgaacaagacagcaaggacagcacctacagcctcagcagcaccctcactttatcaaaagctgactatgagaagcacaaagtttatgcctgtgaagtaactcaccagggcctcagctcccctgtcaccaagtccttcaaccgtggggagtgt

SEQ ID NO: 99 - DNA, 66 bp, Artificial Sequence
  Synthetic nucleic acid
atggacatgagagttcctgcacagctgctggggctcctgctgctctggctctctggtgcccgctgt

SEQ ID NO: 100 - DNA, 708 bp, Artificial Sequence
  Synthetic nucleic acid
atggacatgagagttcctgcacagctgctggggctcctgctgctctggctctctggtgcccgctgtgacatccagatgacacagagcccttccagcctctctgcctcggtgggggacagagtcaccatcacctgtggggcctcggagaacatctatggtgctctgaactggtaccagcagaaacctgggaaggcccccaagctgctcatctatggggccaccaacctggctgatggtgtgccctcgcgcttctctggatctggaagtggcacagacttcaccctcaccatctccagcctgcaacctgaggactttgccacctactactgccagaatgtgctgaacacacccctcacttttggacaagggaccaaagtagaaatcaagaggacagtggcggcgcccagtgtcttcatcttccctccaagcgatgaacaactaaagtctggaacagcctcggtggtctgcctgctgaacaacttctacccaagagaggccaaggtgcagtggaaagttgataatgccctgcagagtggaaacagccaggagagtgtcactgaacaagacagcaaggacagcacctacagcctcagcagcaccctcactttatcaaaagctgactatgagaagcacaaagtttatgcctgtgaagtaactcaccagggcctcagctcccctgtcaccaagtccttcaaccgtggggagtgt

SEQ ID NO: 101 - DNA, 1344 bp, Artificial Sequence
  Synthetic nucleic acid
caagtgcagctggttcaaagtggggctgaagtaaaaaagcctggggcttctgtgaaggtgtcctgcaaggcctcgggctacatcttctccaactactggatccagtgggtccggcaggcccctgggcaaggcctggagtggatgggagagattcttcctggaagtggatccacagagtacacagaaaacttcaaggacagagtcaccatgaccagggacacctccacatccacggtgtacatggaactttccagcctgagatctgaggacactgctgtctactactgtgcccgctacttctttggcagcagccccaattggtattttgatgtctggggccaagggaccttggtcactgtctcctcagcatccacaaaaggaccctcagtcttccctctggccccctgctcccgctccacatcagaaagcacagctgccctgggttgtcttgtaaaagattattttccagaacctgtcactgtgtcctggaacagtggggccctgacttcaggagtccacaccttcccagctgtgctgcagagcagtggcctctattctttatcatctgtggtcactgttccttccagcaactttggcacacagacctacacctgtaatgtggaccacaaaccttccaacacaaaagtggacaaaactgttgaaagaaaatgctgtgtggaatgtccaccctgccctgctccaccagttgctgggcccagtgtcttcctcttccctcccaagcccaaggacaccctcatgatcagccgcaccccagaagtgacctgtgtggtggtggatgtttctcaagaagatcctgaggtgcagtttaattggtatgtcgatggtgttgaagttcataatgccaagacaaaaccccgggaggagcagtttaacagcacctacagagttgtttctgtcctcactgtgctgcaccaggactggctgaatggaaaagaatataaatgcaaagtgagcaacaaaggcctgcccagcagcattgagaagaccatctcaaaggccaagggccagccccgggagccccaagtatatactcttccacccagccaagaagaaatgacaaagaaccaagtatctctcacctgcctggtgaaaggattttatccttctgatattgctgtagaatgggagagtaatggacagccagaaaacaactacaagacgacgccgccggtgctggacagtgatggcagcttcttcctctattcacgcctcactgtggacaagagccgctggcaagaaggaaatgtcttctcctgctctgtcatgcatgaagccctgcacaaccactacacacagaagtccctgagcctctccctggggaag

SEQ ID NO: 102 - DNA, 57 bp, Artificial Sequence
  Synthetic nucleic acid
atgggctggagctgcatcatcctcttcctggtggccacagccacaggtgtccacagc

SEQ ID NO: 103 - DNA, 1401 bp, Artificial Sequence
  Synthetic nucleic acid
atgggctggagctgcatcatcctcttcctggtggccacagccacaggtgtccacagccaagtgcagctggttcaaagtggggctgaagtaaaaaagcctggggcttctgtgaaggtgtcctgcaaggcctcgggctacatcttctccaactactggatccagtgggtccggcaggcccctgggcaaggcctggagtggatgggagagattcttcctggaagtggatccacagagtacacagaaaacttcaaggacagagtcaccatgaccagggacacctccacatccacggtgtacatggaactttccagcctgagatctgaggacactgctgtctactactgtgcccgctacttctttggcagcagccccaattggtattttgatgtctggggccaagggaccttggtcactgtctcctcagcatccacaaaaggaccctcagtcttccctctggccccctgctcccgctccacatcagaaagcacagctgccctgggttgtcttgtaaaagattattttccagaacctgtcactgtgtcctggaacagtggggccctgacttcaggagtccacaccttcccagctgtgctgcagagcagtggcctctattctttatcatctgtggtcactgttccttccagcaactttggcacacagacctacacctgtaatgtggaccacaaaccttccaacacaaaagtggacaaaactgttgaaagaaaatgctgtgtggaatgtccaccctgccctgctccaccagttgctgggcccagtgtcttcctcttccctcccaagcccaaggacaccctcatgatcagccgcaccccagaagtgacctgtgtggtggtggatgtttctcaagaagatcctgaggtgcagtttaattggtatgtcgatggtgttgaagttcataatgccaagacaaaaccccgggaggagcagtttaacagcacctacagagttgtttctgtcctcactgtgctgcaccaggactggctgaatggaaaagaatataaatgcaaagtgagcaacaaaggcctgcccagcagcattgagaagaccatctcaaaggccaagggccagccccgggagccccaagtatatactcttccacccagccaagaagaaatgacaaagaaccaagtatctctcacctgcctggtgaaaggattttatccttctgatattgctgtagaatgggagagtaatggacagccagaaaacaactacaagacgacgccgccggtgctggacagtgatggcagcttcttcctctattcacgcctcactgtggacaagagccgctggcaagaaggaaatgtcttctcctgctctgtcatgcatgaagccctgcacaaccactacacacagaagtccctgagcctctccctggggaag

SEQ ID NO: 104 - DNA, 642 bp, Artificial Sequence
  Synthetic nucleic acid
gatatccagatgacacagagccccagctccttatcagcctcggtgggggacagggtcaccatcacctgtggggcctcggagaacatctatggggctctgaactggtaccagcagaaaccagggaaggcccccaagctgctgatctatggggccaccaacctggctgatggggtgccttcacgcttctctggcagtggcagtggaactgacttcaccctgaccatcagcagcttacaaccagaggattttgccacctactactgccagaatgtcctcaacacacctctcacttttggacaaggaaccaaagtggaaatcaagagaacagtggctgcacccagtgtgttcatcttcccaccaagcgatgaacaactaaagagtggaacagcatctgtggtgtgcctgctcaacaacttctacccaagagaggccaaggtgcagtggaaggttgacaatgccctgcagagtggaaacagtcaagaatctgtcactgaacaagacagcaaggacagcacctacagcctgagctccaccttaactttatcaaaagctgactatgagaagcacaaggtctatgcctgtgaagtgacacaccagggcctctccagccctgtcaccaaatcttttaacagaggagaatgt

SEQ ID NO: 105 - DNA, 66 bp, Artificial Sequence
  Synthetic nucleic acid
atggacatgagagtccctgcacagctgctggggctcctgctgctctggctctctggggcccgctgt

SEQ ID NO: 106 - DNA, 708 bp, Artificial Sequence
  Synthetic nucleic acid
atggacatgagagtccctgcacagctgctggggctcctgctgctctggctctctggggcccgctgtgatatccagatgacacagagccccagctccttatcagcctcggtgggggacagggtcaccatcacctgtggggcctcggagaacatctatggggctctgaactggtaccagcagaaaccagggaaggcccccaagctgctgatctatggggccaccaacctggctgatggggtgccttcacgcttctctggcagtggcagtggaactgacttcaccctgaccatcagcagcttacaaccagaggattttgccacctactactgccagaatgtcctcaacacacctctcacttttggacaaggaaccaaagtggaaatcaagagaacagtggctgcacccagtgtgttcatcttcccaccaagcgatgaacaactaaagagtggaacagcatctgtggtgtgcctgctcaacaacttctacccaagagaggccaaggtgcagtggaaggttgacaatgccctgcagagtggaaacagtcaagaatctgtcactgaacaagacagcaaggacagcacctacagcctgagctccaccttaactttatcaaaagctgactatgagaagcacaaggtctatgcctgtgaagtgacacaccagggcctctccagccctgtcaccaaatcttttaacagaggagaatgt

SEQ ID NO: 107 - DNA, 1344 bp, Artificial Sequence
  Synthetic nucleic acid
caagtgcaattggtccagtcaggtgcagaggttaagaagcctggggcatctgttaaggtttcctgcaaggcatcaggttacattttcagcaactactggattcaatgggtgaggcaggcaccaggtcaaggattggaatggatgggggaaatactgcctgggtcaggatccacagagtacacagagaacttcaaagatagggtcaccatgactagagacacatctactagcactgtttacatggagctcagtagccttaggtcagaagacactgctgtctactactgtgccagatatttctttggcagcagccctaattggtactttgatgtatggggccagggcaccctggtgactgtgagcagtgcttccacaaagggcccatcagtcttcccattggcaccttgtagcaggagcacttcagagagcacagctgcactgggttgcttggtgaaggactacttcccagaaccagtgacagtgtcctggaacagtggtgcacttacatcaggagtgcacaccttccctgcagtactccaatcaagtggcctttacagcctctccagcgttgtcacagtcccctcatctaactttggaactcagacctatacctgtaatgtggaccacaagccttccaacaccaaagtggacaagacagtagaaaggaaatgctgtgtggagtgcccaccgtgcccagccccaccagttgcaggcccaagtgtgttcctcttcccccccaagcccaaagacaccctgatgatcagtaggacccctgaggtgacctgtgttgtggtggatgtgagccaggaggatcctgaggtgcagtttaattggtacgttgacggagtggaagttcataatgccaaaactaagcctagggaggagcagttcaatagcacctacagggtggtgtctgttcttacagtcctgcaccaagactggctgaatggcaaagaatacaagtgcaaagtcagcaacaaggggctgcctagctctattgagaagaccatcagcaaagccaaaggacagcctagagaaccccaggtgtataccttgcctccctcccaagaagagatgaccaagaaccaagtgagcctgacttgccttgtgaagggcttctacccttcagatatagctgttgagtgggagagcaatggccagccagaaaacaactacaaaaccaccccacctgtattggatagtgatggaagctttttcttgtacagcaggctgactgttgataagagcaggtggcaggagggcaatgtgttcagttgttctgtgatgcatgaggccctgcacaatcactacacccagaagagtctgtccctttctctgggcaag

SEQ ID NO: 108 - DNA, 57 bp, Artificial Sequence
  Synthetic nucleic acid
atgggctggtcatgtatcatcctgttcctggtagccactgccacaggggttcatagc

SEQ ID NO: 109 - DNA, 1401 bp, Artificial Sequence
  Synthetic nucleic acid
atgggctggtcatgtatcatcctgttcctggtagccactgccacaggggttcatagccaagtgcaattggtccagtcaggtgcagaggttaagaagcctggggcatctgttaaggtttcctgcaaggcatcaggttacattttcagcaactactggattcaatgggtgaggcaggcaccaggtcaaggattggaatggatgggggaaatactgcctgggtcaggatccacagagtacacagagaacttcaaagatagggtcaccatgactagagacacatctactagcactgtttacatggagctcagtagccttaggtcagaagacactgctgtctactactgtgccagatatttctttggcagcagccctaattggtactttgatgtatggggccagggcaccctggtgactgtgagcagtgcttccacaaagggcccatcagtcttcccattggcaccttgtagcaggagcacttcagagagcacagctgcactgggttgcttggtgaaggactacttcccagaaccagtgacagtgtcctggaacagtggtgcacttacatcaggagtgcacaccttccctgcagtactccaatcaagtggcctttacagcctctccagcgttgtcacagtcccctcatctaactttggaactcagacctatacctgtaatgtggaccacaagccttccaacaccaaagtggacaagacagtagaaaggaaatgctgtgtggagtgcccaccgtgcccagccccaccagttgcaggcccaagtgtgttcctcttcccccccaagcccaaagacaccctgatgatcagtaggacccctgaggtgacctgtgttgtggtggatgtgagccaggaggatcctgaggtgcagtttaattggtacgttgacggagtggaagttcataatgccaaaactaagcctagggaggagcagttcaatagcacctacagggtggtgtctgttcttacagtcctgcaccaagactggctgaatggcaaagaatacaagtgcaaagtcagcaacaaggggctgcctagctctattgagaagaccatcagcaaagccaaaggacagcctagagaaccccaggtgtataccttgcctccctcccaagaagagatgaccaagaaccaagtgagcctgacttgccttgtgaagggcttctacccttcagatatagctgttgagtgggagagcaatggccagccagaaaacaactacaaaaccaccccacctgtattggatagtgatggaagctttttcttgtacagcaggctgactgttgataagagcaggtggcaggagggcaatgtgttcagttgttctgtgatgcatgaggccctgcacaatcactacacccagaagagtctgtccctttctctgggcaag

SEQ ID NO: 110 - DNA, 642 bp, Artificial Sequence
  Synthetic nucleic acid
gatatccagatgacccagagccccagctccctgtctgcatctgtaggtgacagggtcaccattacctgtggagcatcagaaaacatctatggggccttgaactggtatcagcagaagccaggcaaagccccaaagctgttgatatatggtgccaccaacttggcagatggtgtgccaagcagattcagtggatcaggcagtggcacagatttcacactgaccattagcagcctgcaacctgaagactttgctacctactactgccagaatgttctgaacacccccctgacctttggccagggcaccaaggtggagatcaagaggactgttgctgccccttctgtattcatcttcccacccagtgatgagcaattgaagtcaggcactgcatcagtggtgtgtcttcttaacaacttctaccccagagaggccaaggtacaatggaaggttgacaatgcacttcagagtggaaacagccaggagtcagtcactgaacaggacagcaaggatagcacatacagcctgtctagcaccctgactctgagcaaggctgactatgagaagcataaggtgtatgcctgtgaggttacccaccagggactgagcagccctgtgacaaaaagcttcaataggggggagtgc

SEQ ID NO: 111 - DNA, 66 bp, Artificial Sequence
  Synthetic nucleic acid
atggacatgagggtgccagcacagctgctgggcctcctgctgctgtggctgagtggtgcaagatgt

SEQ ID NO: 112 - DNA, 708 bp, Artificial Sequence
  Synthetic nucleic acid
atggacatgagggtgccagcacagctgctgggcctcctgctgctgtggctgagtggtgcaagatgtgatatccagatgacccagagccccagctccctgtctgcatctgtaggtgacagggtcaccattacctgtggagcatcagaaaacatctatggggccttgaactggtatcagcagaagccaggcaaagccccaaagctgttgatatatggtgccaccaacttggcagatggtgtgccaagcagattcagtggatcaggcagtggcacagatttcacactgaccattagcagcctgcaacctgaagactttgctacctactactgccagaatgttctgaacacccccctgacctttggccagggcaccaaggtggagatcaagaggactgttgctgccccttctgtattcatcttcccacccagtgatgagcaattgaagtcaggcactgcatcagtggtgtgtcttcttaacaacttctaccccagagaggccaaggtacaatggaaggttgacaatgcacttcagagtggaaacagccaggagtcagtcactgaacaggacagcaaggatagcacatacagcctgtctagcaccctgactctgagcaaggctgactatgagaagcataaggtgtatgcctgtgaggttacccaccagggactgagcagccctgtgacaaaaagcttcaataggggggagtgc

SEQ ID NO: 113 - DNA, 1344 bp, Artificial Sequence
  Synthetic nucleic acid
caagtacagcttgtacaaagtggcgctgaagtaaaaaagccaggggcttcagtgaaagtgtcctgcaaggcctcgggccacatcttctccaactactggatccagtgggtgcgccaggccccgggccaagggctggagtggatgggagagattttacctggcagtggccacacagagtacacagagaacttcaaggacagagtcaccatgacccgggacaccagcacatcaactgtctacatggagctgagctccctccgatcagaagacacagctgtctactactgtgcccgctacttctttggcagcagccccaattggtattttgatgtgtggggccaaggcaccttggtcaccgtctcctcagcatcaacaaaaggtccttctgtcttccccctggccccctgcagccgcagcacatcagaatccacagcagccctgggctgcctggtaaaagactacttcccagaacctgtcactgtctcctggaacagtggagccctgacatctggtgtccacaccttccctgctgtcctccagagctctggcctctacagcctctcctcagtggtcacagttccatcctccaactttggcacacagacctacacctgcaatgtggaccacaagccttccaacaccaaggtggacaagactgtagagaggaagtgctgtgtggaatgcccaccctgcccagctccacctgttgctgggccttctgtcttcctcttccctccaaagcccaaagacaccctcatgatcagccgcaccccagaagtgacctgtgtggtggtggatgtctcccaggaagaccctgaggtgcagttcaactggtatgtagatggtgttgaagttcataatgccaagaccaagccccgggaggagcagttcaacagcacctacagagtggtgtctgtgctgacagtgctgcaccaggactggctgaatggaaaagaatacaagtgcaaagtttccaacaagggcctgccttcctccattgagaagaccatctccaaagccaagggacagccccgggagccccaagtctacacacttcctccttctcaagaagaaatgacaaagaaccaagtgtctctgacctgcctggtgaagggcttctacccaagtgacattgctgtagaatgggagagcaatggacagcctgaaaacaactacaaaacgaccccacctgtgctggactctgatggcagcttcttcttgtattcacgcctgactgtggacaagagccgctggcaagaaggaaatgtattttcctgctctgtgctccatgaggccctgcacagccactacacccagaagtcactcagcctctctctgggaaaa

SEQ ID NO: 114 - DNA, 1344 bp, Artificial Sequence
  Synthetic nucleic acid
caagtgcagctggttcaaagtggggctgaagtaaaaaagcctggggcttctgtgaaggtgtcctgcaaggcctcgggccacatcttctccaactactggatccagtgggtccggcaggcccctgggcaaggcctggagtggatgggagagattcttcctggaagtggacacacagagtacacagaaaacttcaaggacagagtcaccatgaccagggacacctccacatccacggtgtacatggaactttccagcctgagatctgaggacactgctgtctactactgtgcccgctacttctttggcagcagccccaattggtattttgatgtctggggccaagggaccttggtcactgtctcctcagcatccacaaaaggaccctcagtcttccctctggccccctgctcccgctccacatcagaaagcacagctgccctgggttgtcttgtaaaagattattttccagaacctgtcactgtgtcctggaacagtggggccctgacttcaggagtccacaccttcccagctgtgctgcagagcagtggcctctattctttatcatctgtggtcactgttccttccagcaactttggcacacagacctacacctgtaatgtggaccacaaaccttccaacacaaaagtggacaaaactgttgaaagaaaatgctgtgtggaatgtccaccctgccctgctccaccagttgctgggcccagtgtcttcctcttccctcccaagcccaaggacaccctcatgatcagccgcaccccagaagtgacctgtgtggtggtggatgtttctcaagaagatcctgaggtgcagtttaattggtatgtcgatggtgttgaagttcataatgccaagacaaaaccccgggaggagcagtttaacagcacctacagagttgtttctgtcctcactgtgctgcaccaggactggctgaatggaaaagaatataaatgcaaagtgagcaacaaaggcctgcccagcagcattgagaagaccatctcaaaggccaagggccagccccgggagccccaagtatatactcttccacccagccaagaagaaatgacaaagaaccaagtatctctcacctgcctggtgaaaggattttatccttctgatattgctgtagaatgggagagtaatggacagccagaaaacaactacaagacgacgccgccggtgctggacagtgatggcagcttcttcctctattcacgcctcactgtggacaagagccgctggcaagaaggaaatgtcttctcctgctctgtcctccatgaagccctgcacagccactacacacagaagtccctgagcctctccctggggaag

SEQ ID NO: 115 - DNA, 1344 bp, Artificial Sequence
  Synthetic nucleic acid
caagtgcaattggtccagtcaggtgcagaggttaagaagcctggggcatctgttaaggtttcctgcaaggcatcaggtcacattttcagcaactactggattcaatgggtgaggcaggcaccaggtcaaggattggaatggatgggggaaatactgcctgggtcaggacacacagagtacacagagaacttcaaagatagggtcaccatgactagagacacatctactagcactgtttacatggagctcagtagccttaggtcagaagacactgctgtctactactgtgccagatatttctttggcagcagccctaattggtactttgatgtatggggccagggcaccctggtgactgtgagcagtgcttccacaaagggcccatcagtcttcccattggcaccttgtagcaggagcacttcagagagcacagctgcactgggttgcttggtgaaggactacttcccagaaccagtgacagtgtcctggaacagtggtgcacttacatcaggagtgcacaccttccctgcagtactccaatcaagtggcctttacagcctctccagcgttgtcacagtcccctcatctaactttggaactcagacctatacctgtaatgtggaccacaagccttccaacaccaaagtggacaagacagtagaaaggaaatgctgtgtggagtgcccaccgtgcccagccccaccagttgcaggcccaagtgtgttcctcttcccccccaagcccaaagacaccctgatgatcagtaggacccctgaggtgacctgtgttgtggtggatgtgagccaggaggatcctgaggtgcagtttaattggtacgttgacggagtggaagttcataatgccaaaactaagcctagggaggagcagttcaatagcacctacagggtggtgtctgttcttacagtcctgcaccaagactggctgaatggcaaagaatacaagtgcaaagtcagcaacaaggggctgcctagctctattgagaagaccatcagcaaagccaaaggacagcctagagaaccccaggtgtataccttgcctccctcccaagaagagatgaccaagaaccaagtgagcctgacttgccttgtgaagggcttctacccttcagatatagctgttgagtgggagagcaatggccagccagaaaacaactacaaaaccaccccacctgtattggatagtgatggaagctttttcttgtacagcaggctgactgttgataagagcaggtggcaggagggcaatgtgttcagttgttctgtgctccatgaggccctgcacagccactacacccagaagagtctgtccctttctctgggcaag

SEQ ID NO: 116 - DNA, 410 bp, Artificial Sequence
  Synthetic nucleic acid
tgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataa

SEQ ID NO: 117 - DNA, 222 bp, Artificial Sequence
  Synthetic nucleic acid
ctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagagat

SEQ ID NO: 118 - DNA, 402 bp, Artificial Sequence
  Synthetic nucleic acid
accagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgtaagtatgcctt

SEQ ID NO: 119 - DNA, 202 bp, Artificial Sequence
  Synthetic nucleic acid
acctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatc

SEQ ID NO: 120 - DNA, 392 bp, Artificial Sequence
  Synthetic nucleic acid
ttctgatatctatttaactgatttcacccaaatgctttgaacctgggaatgtacctctccccctcccccacccccaacaggagtgagacaagggccagggctattgcccctgctgactcaatattggctaatcactgcctagaactgataaggtgatcaaatgaccaggtgccttcaacctttaccctggtagaagcctcttattcacctcttttcctgccagagccctccattgggaggggacgggcggaagctgttttctgaatttgttttactgggggtagggtatgttcagtgatcgtccctgtcacctgacagggggtgggtaaacagacaggtatatagccccttcctctccagccagggcaggcacagacaccaaggacagagac

SEQ ID NO: 121 - DNA, 210 bp, Artificial Sequence
  Synthetic nucleic acid
agttccagatggtaaatatacacaagggatttagtcaaacaattttttggcaagaatattatgaattttgtaatcggttggcagccaatgaaatacaaagatgagtctagttaataatctacaattattggttaaagaagtatattagtgctaatttccctccgtttgtcctagcttttctcttctgtcaaccccacacgcctttggcac

SEQ ID NO: 122 - DNA, 113 bp, Artificial Sequence
  Synthetic nucleic acid
tgttgcttaaatgtttgttgactaagtcaataatcagaatcagcaaattaaatatttaactaaggaaactaggcaaggttcatatttattcctagcagaggactcagatataa

SEQ ID NO: 123 - DNA, 325 bp, Artificial Sequence
  Synthetic nucleic acid
ctgcaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggg

SEQ ID NO: 124 - DNA, 299 bp, Artificial Sequence
  Synthetic nucleic acid
gtttttgggcccgccctgcccccttccgacctcttagttcctatcctccagcagctgtttgtgtgctgcctctgaagtccaccctgaatgaccttcagcctgttcccgtccctgatatgggcaaacattgcaagcagcaaacagcaaacacatagccctccctgcgtgctgaccttggagctgcggcagaggtcagagacctctcagggcccataccacttccaacatccccttgatctcttggattttggtggagaggggcagaggttgtcctggcctggttaggtagtgtgagaggg

SEQ ID NO: 125 - DNA, 54 bp, Artificial Sequence
  Synthetic nucleic acid
gaaggcagaggcagcctgctgacctgtggagatgtggaagagaacccaggccct

SEQ ID NO: 126 - DNA, 9 bp, Artificial Sequence
  Synthetic nucleic acid
ggctctgga

SEQ ID NO: 127 - DNA, 75 bp, Artificial Sequence
  Synthetic nucleic acid
agaaagagaagaggctctggagaaggcagaggcagcctgctgacctgtggagatgtggaagagaacccaggccct

SEQ ID NO: 128 - DNA, 57 bp, Artificial Sequence
  Synthetic nucleic acid
gctactaacttcagcctgctgaagcaggctggagatgtggaggagaaccctggacct

SEQ ID NO: 129 - DNA, 78 bp, Artificial Sequence
  Synthetic nucleic acid
agaaagagaagaggctctggagctactaacttcagcctgctgaagcaggctggagatgtggaggagaaccctggacct

SEQ ID NO: 130 - DNA, 66 bp, Artificial Sequence
  Synthetic nucleic acid
atggacatgagggtgccagcacagctgctgggcctcctgctgctgtggctgagtggtgcaaggtgt

SEQ ID NO: 131 - DNA, 642 bp, Artificial Sequence
  Synthetic nucleic acid
gatatccagatgacccagagccccagctccctgtctgcatctgtaggtgacagggtcaccattacctgtggagcatcagaaaacatctatggggccttgaactggtatcagcagaagccaggtaaagccccaaagctgttgatatatggtgccaccaacttggcagatggtgtgccaagcagattcagtggatcaggcagtggcacagatttcacactgaccattagcagcctgcaacctgaagactttgctacctactactgccagaatgttctgaacacccccctgacctttggccagggcaccaaggtggagatcaagaggactgttgctgccccttctgtattcatcttcccacccagtgatgagcaattgaagtcaggcactgcatcagtggtgtgtcttcttaacaacttctaccccagagaggccaaggtacaatggaaggttgacaatgcacttcagagtggaaacagccaggagtcagtcactgaacaggacagcaaggatagcacatacagcctgtctagcaccctgactctgagcaaggctgactatgagaagcataaggtgtatgcctgtgaggttacccaccagggactgagcagccctgtgacaaaaagcttcaataggggggagtgc

SEQ ID NO: 132 - DNA, 708 bp, Artificial Sequence
  Synthetic nucleic acid
atggacatgagggtgccagcacagctgctgggcctcctgctgctgtggctgagtggtgcaaggtgtgatatccagatgacccagagccccagctccctgtctgcatctgtaggtgacagggtcaccattacctgtggagcatcagaaaacatctatggggccttgaactggtatcagcagaagccaggtaaagccccaaagctgttgatatatggtgccaccaacttggcagatggtgtgccaagcagattcagtggatcaggcagtggcacagatttcacactgaccattagcagcctgcaacctgaagactttgctacctactactgccagaatgttctgaacacccccctgacctttggccagggcaccaaggtggagatcaagaggactgttgctgccccttctgtattcatcttcccacccagtgatgagcaattgaagtcaggcactgcatcagtggtgtgtcttcttaacaacttctaccccagagaggccaaggtacaatggaaggttgacaatgcacttcagagtggaaacagccaggagtcagtcactgaacaggacagcaaggatagcacatacagcctgtctagcaccctgactctgagcaaggctgactatgagaagcataaggtgtatgcctgtgaggttacccaccagggactgagcagccctgtgacaaaaagcttcaataggggggagtgc

(19) United States
(12) Patent Application Publication
(10) Pub. No.: US 2022/0106611 A1
(43) Pub. Date: Apr. 7, 2022

(54) VECTORIZED ANTIBODIES AND USES THEREOF

(71) Applicant: HOMOLOGY MEDICINES, INC., BEDFORD, MA (US)

(72) Inventors: Carmen BARNES, Bedford, MA (US); Yogeshwar SHARMA, Bedford, MA (US); Serena DOLLIVE, Bedford, MA (US); Omar FRANCONE, Bedford, MA (US)

(21) Appl. No.: 17/447,231

(22) Filed: Sep. 9, 2021

Related U.S. Application Data
(60) Provisional application No. 63/179,990, filed on Apr. 26, 2021.
(60) Provisional application No. 63/075,898, filed on Sep. 9, 2020.

(51) Int. Cl. C12N 15/86; C07K 16/10; A61K 35/761

(52) U.S. Cl. CPC C12N 15/86; C07K 16/1036; C12N 2710/10032; C12N 2710/10022; A61K 35/761

(57) ABSTRACT

Provided are recombinant adeno-associated virus (rAAV) compositions for the expression of antibodies (e.g., anti-complement component 5 (C5) antibodies) in cells, and methods of treating disorders with the same (e.g., disorders associated with C5 activity (e.g., Paroxysmal Nocturnal Hemoglobinuria)). Also provided are compositions, systems and methods for making the rAAV compositions.

### RELATED APPLICATIONS

This application claims priority to U.S. Provisional Patent Application Serial Nos. 63/075,898, filed Sep. 9, 2020, and 63/179,990, filed Apr. 26, 2021, the entire disclosures of which are hereby incorporated herein by reference.

### SEQUENCE LISTING

This application contains a sequence listing which has been submitted electronically in ASCII format and is hereby incorporated by reference in its entirety (said ASCII copy, created on Sep. 8, 2021, is named “404217-HMW-042US_185754_ST25.txt” and is 252,035 bytes in size).

### BACKGROUND

Therapeutic antibodies represent a potent class of drugs, possessing high specificity to a target of interest. However, many antibodies require large individual doses and regular administration to achieve the desired therapeutic effect. This is especially true for antibody targets that are found at high concentrations in a patient's serum. For example, anti-complement component 5 (C5) antibodies used of the treatment of C5-mediated diseases, such as paroxysmal nocturnal hemoglobinuria (PNH), neuromyelitis optica spectrum disorder (NMOSD), and atypical hemolytic uremic syndrome (aHUS), require multiple large doses of the antibody due to the high abundance of C5 in serum.

Viral delivery mechanisms offer an attractive alternative to conventional antibody treatments, especially for antibody targets that are found at high concentrations in a patient's serum. In particular, a single administration of a viral vector harboring expression cassettes for antibody heavy and light chains has the potential to produce sustained therapeutic levels of an antibody in the serum of a subject, thereby bypassing the need for continual administration of high dose antibody.

Accordingly, there is a need in the art for improved viral vectors for the efficient and sustained expression of antibodies in a subject.

### SUMMARY

Provided herein are recombinant adeno-associated virus (rAAV) compositions for the expression of antibodies (e.g., anti-complement component 5 (C5) antibodies) in cells, and methods for using the same to treat disorders (e.g., disorders associated with C5 activity (e.g., Paroxysmal Nocturnal Hemoglobinuria)). Also provided are compositions, systems and methods for making the rAAV compositions.

Accordingly, in one aspect, the disclosure provides a recombinant adeno-associated virus (rAAV) genome comprising:

(a) a first expression cassette comprising, from 5′ to 3′,
a first liver-specific transcriptional regulatory element,
a first coding sequence encoding a first polypeptide comprising an antibody heavy chain operably linked to a first signal sequence, and
a first polyadenylation sequence; and
(b) a second expression cassette comprising, from 5′ to 3′,
a second liver-specific transcriptional regulatory element,
a second coding sequence encoding a second polypeptide comprising an antibody light chain operably linked to a second signal sequence, and
a second polyadenylation sequence,
wherein expression of the first and second coding sequences produces an antibody comprising the antibody heavy chain and the antibody light chain.

In certain embodiments, the first and/or second transcriptional regulatory element comprise a promoter element selected from the group consisting of human albumin promoter, a human transthyretin (TTR) promoter, a human thyroxine binding globulin (TBG) promoter, a human ApoH promoter, a human SERPINA1 (hAAT) promoter, and a hepatic specific regulatory module thereof, such as a human ApoE/C-I hepatic control region (HCR) 1 or 2.

In certain embodiments, the first and/or second transcriptional regulatory element comprise a promoter element comprising a nucleic acid sequence at least 90% identical to a sequence selected from the group consisting of SEQ ID NO: 25, 27, 66, 68, 69, 116, and 117.

In certain embodiments, the transcriptional regulatory element comprises a nucleotide sequence at least 90% identical to the nucleotide sequence set forth in SEQ ID NO: 27. In certain embodiments, the transcriptional regulatory element comprises the nucleotide sequence set forth in SEQ ID NO: 27. In certain embodiments, the nucleotide sequence of the transcriptional regulatory element consists of the nucleotide sequence set forth in SEQ ID NO: 27.

In certain embodiments, the transcriptional regulatory element comprises a nucleotide sequence at least 90% identical to the nucleotide sequence set forth in SEQ ID NO: 67. In certain embodiments, the transcriptional regulatory element comprises the nucleotide sequence set forth in SEQ ID NO: 67. In certain embodiments, the nucleotide sequence of the transcriptional regulatory element consists of the nucleotide sequence set forth in SEQ ID NO: 67.

In certain embodiments, the first and/or second expression cassette further comprise an intron element positioned 5′ to the first and/or second coding sequence and 3′ to the transcriptional regulatory element.

In certain embodiments, the intron element is an exogenous intron element, optionally wherein the exogenous intron element is an SV40 intron element or a minute virus of mouse (MVM) intron element.

In certain embodiments, the SV40 intron element comprises a nucleotide sequence at least 90% identical to the nucleotide sequence set forth in SEQ ID NO: 29. In certain embodiments, the SV40 intron element comprises the nucleotide sequence set forth in SEQ ID NO: 29. In certain embodiments, the nucleotide sequence of the SV40 intron element consists of the nucleotide sequence set forth in SEQ ID NO: 29.

In certain embodiments, the MVM intron element comprises a nucleotide sequence at least 90% identical to the nucleotide sequence set forth in SEQ ID NO: 30. In certain embodiments, the MVM intron element comprises the nucleotide sequence set forth in SEQ ID NO: 30. In certain embodiments, the nucleotide sequence of the MVM intron element consists of the nucleotide sequence set forth in SEQ ID NO: 30.

In certain embodiments, the first and second transcriptional regulatory element are identical.

In certain embodiments, the first transcriptional regulatory element comprises an HCR 1 element, a hAAT promoter, and an SV40 intron element, and the second transcriptional regulatory element comprises a SERPINA1 hepatic specific regulatory module, a TTR promoter, and an MVM intron element.

In certain embodiments, the first transcriptional regulatory element comprises the nucleic acid sequence of SEQ ID NO: 50 and the second transcriptional regulatory element comprises the nucleic acid sequence of SEQ ID NO: 43.

In certain embodiments, the first and/or second expression cassette further comprise a polyadenylation sequence 3′ to the first and/or second coding sequence.

In certain embodiments, the polyadenylation sequence is an exogenous polyadenylation sequence, optionally wherein the exogenous polyadenylation sequence is an SV40 polyadenylation sequence, or a bovine growth hormone (BGH) polyadenylation sequence.

In certain embodiments, the SV40 polyadenylation sequence comprises a nucleotide sequence at least 90% identical to the nucleotide sequence set forth in SEQ ID NO: 31. In certain embodiments, the SV40 polyadenylation sequence comprises the nucleotide sequence set forth in SEQ ID NO: 31. In certain embodiments, the nucleotide sequence of the SV40 polyadenylation sequence consists of the nucleotide sequence set forth in SEQ ID NO: 31.

In certain embodiments, the BGH polyadenylation sequence comprises a nucleotide sequence at least 90% identical to the nucleotide sequence set forth in SEQ ID NO: 33. In certain embodiments, the BGH polyadenylation sequence comprises the nucleotide sequence set forth in SEQ ID NO: 33. In certain embodiments, the nucleotide sequence of the BGH polyadenylation sequence consists of the nucleotide sequence set forth in SEQ ID NO: 33.

In certain embodiments, the first and second expression cassette comprise identical polyadenylation sequences.

In certain embodiments, the first expression cassette comprises the SV40 polyadenylation sequence. In certain embodiments, the second expression cassette comprises the BGH polyadenylation sequence.

In certain embodiments, the first polyadenylation sequence comprises the nucleic acid sequence of SEQ ID NO: 31 and the second polyadenylation sequence comprises the nucleic acid sequence of SEQ ID NO: 33.

In certain embodiments, the first and second expression cassettes are in the same orientation in the rAAV genome. In certain embodiments, the first and second expression cassettes are in opposite orientations in the rAAV genome.

In certain embodiments, the first and second expression cassettes are in opposite orientations, with the first and second polyadenylation sequences distally positioned in the rAAV genome.

In certain embodiments, the rAAV genome further comprises a stuffer sequence interposed between the first and second transcriptional regulatory elements.

In certain embodiments, the stuffer sequence comprises a beta globin polyadenylation sequence. In certain embodiments, the beta globin polyadenylation sequence comprises the nucleic acid sequence of SEQ ID NO: 51.

In certain embodiments, the rAAV genome comprises from 5′ to 3′: (a) the first polyadenylation sequence comprising the nucleic acid sequence of SEQ ID NO: 33; (b) the first coding sequence; (c) the first liver-specific transcriptional regulatory element comprising the nucleic acid sequence of SEQ ID NO: 27; (d) a stuffer sequence comprising the nucleic acid sequence of SEQ ID NO: 51; (e) the second liver-specific transcriptional regulatory element comprising the nucleic acid sequence of SEQ ID NO: 67; (0 the second coding sequence; (g) the second transcriptional polyadenylation sequence comprising the nucleic acid sequence of SEQ ID NO: 31.

In certain embodiments, the rAAV genome comprises from 5′ to 3′: the reverse complement of the first expression cassette; a stuffer sequence; and the second expression cassette.

In certain embodiments, the rAAV genome comprises: (a) the first expression cassette comprises, from 5′ to 3′: a nucleotide sequence at least 90% identical to the nucleotide sequence set forth in SEQ ID NO: 27, the first coding sequence, a nucleotide sequence at least 90% identical to the nucleotide sequence set forth in SEQ ID NO: 33; (b) the stuffer sequence comprising a nucleotide sequence at least 90% identical to the nucleotide sequence set forth in SEQ ID NO: 51 or the reverse complement thereof; and (c) the second expression cassette comprising, from 5′ to 3′, a nucleotide sequence at least 90% identical to the nucleotide sequence set forth in SEQ ID NO: 67, the second coding sequence, a nucleotide sequence at least 90% identical to the nucleotide sequence set forth in SEQ ID NO: 31.

In certain embodiments, the rAAV genome comprises: (a) the first expression cassette comprises, from 5′ to 3′: a nucleotide sequence at least 90% identical to the nucleotide sequence set forth in SEQ ID NO: 67, the first coding sequence, a nucleotide sequence at least 90% identical to the nucleotide sequence set forth in SEQ ID NO: 31; (b) the stuffer sequence comprising a nucleotide sequence at least 90% identical to the nucleotide sequence set forth in SEQ ID NO: 51 or the reverse complement thereof; and (c) the second expression cassette comprising, from 5′ to 3′, a nucleotide sequence at least 90% identical to the nucleotide sequence set forth in SEQ ID NO: 27, the second coding sequence, a nucleotide sequence at least 90% identical to the nucleotide sequence set forth in SEQ ID NO: 33.

In certain embodiments, the rAAV genome comprises: (a) the first expression cassette comprises, from 5′ to 3′: a nucleotide sequence at least 90% identical to the nucleotide sequence set forth in SEQ ID NO: 25, a nucleotide sequence at least 90% identical to the nucleotide sequence set forth in SEQ ID NO: 26, the first coding sequence, the first polyadenylation sequence; (b) the stuffer sequence comprising a nucleotide sequence at least 90% identical to the nucleotide sequence set forth in SEQ ID NO: 51 or the reverse complement thereof; and (c) the second expression cassette comprising, from 5′ to 3′, a nucleotide sequence at least 90% identical to the nucleotide sequence set forth in SEQ ID NO: 119, a nucleotide sequence at least 90% identical to the nucleotide sequence set forth in SEQ ID NO: 45, the second coding sequence, a nucleotide sequence at least 90% identical to the nucleotide sequence set forth in SEQ ID NO: 31.

In certain embodiments, the rAAV genome comprises: (a) the first expression cassette comprises, from 5′ to 3′: a nucleotide sequence at least 90% identical to the nucleotide sequence set forth in SEQ ID NO: 119, a nucleotide sequence at least 90% identical to the nucleotide sequence set forth in SEQ ID NO: 45, the first coding sequence, a nucleotide sequence at least 90% identical to the nucleotide sequence set forth in SEQ ID NO: 31; (b) the stuffer sequence comprising a nucleotide sequence at least 90% identical to the nucleotide sequence set forth in SEQ ID NO: 51 or the reverse complement thereof; and (c) the second expression cassette comprising, from 5′ to 3′, a nucleotide sequence at least 90% identical to the nucleotide sequence set forth in SEQ ID NO: 25, a nucleotide sequence at least 90% identical to the nucleotide sequence set forth in SEQ ID NO: 26, the second coding sequence, the first polyadenylation sequence.

In certain embodiments, the rAAV genome comprises: (a) the first expression cassette comprises, from 5′ to 3′: the nucleotide sequence set forth in SEQ ID NO: 27, the first coding sequence, the nucleotide sequence set forth in SEQ ID NO: 33; (b) the stuffer sequence comprising the nucleotide sequence set forth in SEQ ID NO: 51 or the reverse complement thereof; and (c) the second expression cassette comprising, from 5′ to 3′, the nucleotide sequence set forth in SEQ ID NO: 67, the second coding sequence, the nucleotide sequence set forth in SEQ ID NO: 31.

In certain embodiments, the rAAV genome comprises: (a) the first expression cassette comprises, from 5′ to 3′: the nucleotide sequence set forth in SEQ ID NO: 67, the first coding sequence, the nucleotide sequence set forth in SEQ ID NO: 31; (b) the stuffer sequence comprising the nucleotide sequence set forth in SEQ ID NO: 51 or the reverse complement thereof; and (c) the second expression cassette comprising, from 5′ to 3′, the nucleotide sequence set forth in SEQ ID NO: 27, the second coding sequence, the nucleotide sequence set forth in SEQ ID NO: 33.

In certain embodiments, the rAAV genome comprises: (a) the first expression cassette comprises, from 5′ to 3′: the nucleotide sequence set forth in SEQ ID NO: 25, the nucleotide sequence set forth in SEQ ID NO: 26, the first coding sequence, the first polyadenylation sequence; (b) the stuffer sequence comprising the nucleotide sequence set forth in SEQ ID NO: 51 or the reverse complement thereof; and (c) the second expression cassette comprising, from 5′ to 3′, the nucleotide sequence set forth in SEQ ID NO: 119, the nucleotide sequence set forth in SEQ ID NO: 45, the second coding sequence, the nucleotide sequence set forth in SEQ ID NO: 31.

In certain embodiments, the rAAV genome comprises: (a) the first expression cassette comprises, from 5′ to 3′: the nucleotide sequence set forth in SEQ ID NO: 119, the nucleotide sequence set forth in SEQ ID NO: 45, the first coding sequence, the nucleotide sequence set forth in SEQ ID NO: 31; (b) the stuffer sequence comprising the nucleotide sequence set forth in SEQ ID NO: 51 or the reverse complement thereof; and (c) the second expression cassette comprising, from 5′ to 3′, the nucleotide sequence set forth in SEQ ID NO: 25, the nucleotide sequence set forth in SEQ ID NO: 26, the second coding sequence, the first polyadenylation sequence.

In one aspect, the disclosure provides an rAAV genome comprising a bicistronic expression cassette comprising, from 5′ to 3′:

(a) a liver-specific transcriptional regulatory element; a first coding sequence encoding a first polypeptide comprising an antibody heavy chain operably linked to a first signal sequence; a ribosomal skipping sequence encoding a ribosomal skipping peptide; a second coding sequence encoding a second polypeptide comprising an antibody light chain operably linked to a second signal sequence; and a polyadenylation sequence, or
(b) a liver-specific transcriptional regulatory element; a second coding sequence encoding a second polypeptide comprising an antibody light chain operably linked to a second signal sequence; a ribosomal skipping sequence encoding a ribosomal skipping peptide; a first coding sequence encoding a first polypeptide comprising an antibody heavy chain operably linked to a first signal sequence; and a polyadenylation sequence,
wherein expression of the bicistronic expression cassette produces an antibody comprising the antibody heavy chain and the antibody light chain.

In certain embodiments, the transcriptional regulatory element comprises a promoter element selected from the group consisting of human albumin promoter, a human transthyretin (TTR) promoter, the human thyroxine binding globulin (TBG) promoter, a human ApoH promoter, a human SERPINA1 (hAAT) promoter, and a hepatic specific regulatory module thereof, such as a human ApoE/C-I hepatic control region (HCR) 1 or 2.

In certain embodiments, the transcriptional regulatory element comprises a promoter element comprising a nucleic acid sequence at least 90% identical to a sequence selected from the group consisting of SEQ ID NO: 25, 27, 66, 68, 69, 116, and 117.

In certain embodiments, the transcriptional regulatory element comprises a nucleotide sequence at least 90% identical to the nucleotide sequence set forth in SEQ ID NO: 27. In certain embodiments, the transcriptional regulatory element comprises the nucleotide sequence set forth in SEQ ID NO: 27. In certain embodiments, the nucleotide sequence of the transcriptional regulatory element consists of the nucleotide sequence set forth in SEQ ID NO: 27.

In certain embodiments, the transcriptional regulatory element comprises a nucleotide sequence at least 90% identical to the nucleotide sequence set forth in SEQ ID NO: 67. In certain embodiments, the transcriptional regulatory element comprises the nucleotide sequence set forth in SEQ ID NO: 67. In certain embodiments, the nucleotide sequence of the transcriptional regulatory element consists of the nucleotide sequence set forth in SEQ ID NO: 67.

In certain embodiments, the bicistronic expression cassette further comprises an intron element positioned 5′ to the first and/or second coding sequence and 3′ to the transcriptional regulatory element.

In certain embodiments, the intron element is an exogenous intron element, optionally wherein the exogenous intron element is an SV40 intron element or a minute virus of mouse (MVM) intron element.

In certain embodiments, the SV40 intron element comprises a nucleotide sequence at least 90% identical to the nucleotide sequence set forth in SEQ ID NO: 29. In certain embodiments, the SV40 intron element comprises the nucleotide sequence set forth in SEQ ID NO: 29. In certain embodiments, the nucleotide sequence of the SV40 intron element consists of the nucleotide sequence set forth in SEQ ID NO: 29.

In certain embodiments, the MVM intron element comprises a nucleotide sequence at least 90% identical to the nucleotide sequence set forth in SEQ ID NO: 30. In certain embodiments, the MVM intron element comprises the nucleotide sequence set forth in SEQ ID NO: 30. In certain embodiments, the nucleotide sequence of the MVM intron element consists of the nucleotide sequence set forth in SEQ ID NO: 30.

In certain embodiments, the transcriptional regulatory element comprises:

a) an HCR 1 element, a hAAT promoter, and an SV40 intron element; or
b) a SERPINA1 hepatic specific regulatory module, a TTR promoter, and an MVM intron element.

In certain embodiments, the transcriptional regulatory element comprises the nucleic acid sequence of SEQ ID NO: 50, or the nucleic acid sequence of SEQ ID NO: 43.

In certain embodiments, the polyadenylation sequence is an exogenous polyadenylation sequence, optionally wherein the exogenous polyadenylation sequence is an SV40 polyadenylation sequence, or a bovine growth hormone (BGH) polyadenylation sequence.

In certain embodiments, the SV40 polyadenylation sequence comprises a nucleotide sequence at least 90% identical to the nucleotide sequence set forth in SEQ ID NO: 31. In certain embodiments, the SV40 polyadenylation sequence comprises the nucleotide sequence set forth in SEQ ID NO: 31. In certain embodiments, the nucleotide sequence of the SV40 polyadenylation sequence consists of the nucleotide sequence set forth in SEQ ID NO: 31.

In certain embodiments, the BGH polyadenylation sequence comprises a nucleotide sequence at least 90% identical to the nucleotide sequence set forth in SEQ ID NO: 33. In certain embodiments, the BGH polyadenylation sequence comprises the nucleotide sequence set forth in SEQ ID NO: 33. In certain embodiments, the nucleotide sequence of the BGH polyadenylation sequence consists of the nucleotide sequence set forth in SEQ ID NO: 33.

In certain embodiments, the first and/or second signal sequence is a naturally occurring signal sequence. In certain embodiments, the first and/or second signal sequence is an antibody signal sequence, optionally a human IgG2 or IgK signal sequence. In certain embodiments, the first and/or second signal sequence is a non-naturally occurring signal sequence. In certain embodiments, the first and/or second signal sequence comprises the amino acid sequence of SEQ ID NO: 80. In certain embodiments, the first and/or second signal sequence comprises the amino acid sequence of SEQ ID NO: 81. In certain embodiments, the first signal sequence comprises the amino acid sequence of SEQ ID NO: 80 and the second signal sequence comprises the amino acid sequence of SEQ ID NO: 81. In certain embodiments, the first and/or second coding sequence comprises any one of the nucleic acid sequences set forth in SEQ ID NO: 23, 96, 102, or 108. In certain embodiments, the first and/or second coding sequence comprises any one of the nucleic acid sequences set forth in SEQ ID NO: 24, 99, 105, 111, or 130. In certain embodiments, the first coding sequence comprises any one of the nucleic acid sequences set forth in SEQ ID NO: 23, 96, 102, or 108 and the second coding sequence comprises any one of the nucleic acid sequences set forth in SEQ ID NO: 24, 99, 105, 111, or 130.

In certain embodiments, the antibody specifically binds to complement C5.

In certain embodiments, the antibody heavy chain comprises the amino acid sequence of SEQ ID NO: 64. In certain embodiments, the antibody heavy chain comprises the amino acid sequence of SEQ ID NO: 82. In certain embodiments, the antibody light chain comprises the amino acid sequence of SEQ ID NO: 77.

In certain embodiments, the first and/or second coding sequence has been optimized for expression in human cells.

In certain embodiments, the first coding sequence comprises any one of the nucleic acid sequences set forth in SEQ ID NO: 52, 113, 114, or 115.

In certain embodiments, the first coding sequence comprises any one of the nucleic acid sequences set forth in SEQ ID NO: 83, 94, 95, 101, or 107.

In certain embodiments, the second coding sequence comprises any one of the nucleic acid sequences set forth in SEQ ID NO: 53, 98, 104, 110, or 131.

In certain embodiments, the first coding sequence comprises a nucleotide sequence selected from the group consisting of SEQ ID NO: 52, 62, 83, 94, 95, 96, 97, 101, 102, 103, 107, 108, 109, 113, 114, and 115, and the second coding sequence comprises the nucleotide sequence set forth in SEQ ID NO: 53.

In certain embodiments, the first coding sequence comprises a nucleotide sequence selected from the group consisting of SEQ ID NO: 52, 62, 83, 94, 95, 96, 97, 101, 102, 103, 107, 108, 109, 113, 114, and 115, and the second coding sequence comprises the nucleotide sequence set forth in SEQ ID NO: 63.

In certain embodiments, the first coding sequence comprises a nucleotide sequence selected from the group consisting of SEQ ID NO: 52, 62, 83, 94, 95, 96, 97, 101, 102, 103, 107, 108, 109, 113, 114, and 115, and the second coding sequence comprises the nucleotide sequence set forth in SEQ ID NO: 98.

In certain embodiments, the first coding sequence comprises a nucleotide sequence selected from the group consisting of SEQ ID NO: 52, 62, 83, 94, 95, 96, 97, 101, 102, 103, 107, 108, 109, 113, 114, and 115, and the second coding sequence comprises the nucleotide sequence set forth in SEQ ID NO: 99.

In certain embodiments, the first coding sequence comprises a nucleotide sequence selected from the group consisting of SEQ ID NO: 52, 62, 83, 94, 95, 96, 97, 101, 102, 103, 107, 108, 109, 113, 114, and 115, and the second coding sequence comprises the nucleotide sequence set forth in SEQ ID NO: 100.

In certain embodiments, the first coding sequence comprises a nucleotide sequence selected from the group consisting of SEQ ID NO: 52, 62, 83, 94, 95, 96, 97, 101, 102, 103, 107, 108, 109, 113, 114, and 115, and the second coding sequence comprises the nucleotide sequence set forth in SEQ ID NO: 104.

In certain embodiments, the first coding sequence comprises a nucleotide sequence selected from the group consisting of SEQ ID NO: 52, 62, 83, 94, 95, 96, 97, 101, 102, 103, 107, 108, 109, 113, 114, and 115, and the second coding sequence comprises the nucleotide sequence set forth in SEQ ID NO: 105.

In certain embodiments, the first coding sequence comprises a nucleotide sequence selected from the group consisting of SEQ ID NO: 52, 62, 83, 94, 95, 96, 97, 101, 102, 103, 107, 108, 109, 113, 114, and 115, and the second coding sequence comprises the nucleotide sequence set forth in SEQ ID NO: 106.

In certain embodiments, the first coding sequence comprises a nucleotide sequence selected from the group consisting of SEQ ID NO: 52, 62, 83, 94, 95, 96, 97, 101, 102, 103, 107, 108, 109, 113, 114, and 115, and the second coding sequence comprises the nucleotide sequence set forth in SEQ ID NO: 110.

In certain embodiments, the first coding sequence comprises a nucleotide sequence selected from the group consisting of SEQ ID NO: 52, 62, 83, 94, 95, 96, 97, 101, 102, 103, 107, 108, 109, 113, 114, and 115, and the second coding sequence comprises the nucleotide sequence set forth in SEQ ID NO: 111.

In certain embodiments, the first coding sequence comprises a nucleotide sequence selected from the group consisting of SEQ ID NO: 52, 62, 83, 94, 95, 96, 97, 101, 102, 103, 107, 108, 109, 113, 114, and 115, and the second coding sequence comprises the nucleotide sequence set forth in SEQ ID NO: 112.

In certain embodiments, the rAAV genome is a single stranded rAAV genome.

In certain embodiments, the rAAV genome is a self-complementary rAAV genome.

In certain embodiments, the rAAV genome comprises the nucleic acid sequence of SEQ ID NO: 84. In certain embodiments, the rAAV genome comprises the nucleic acid sequence of SEQ ID NO: 85. In certain embodiments, the rAAV genome comprises the nucleic acid sequence of SEQ ID NO: 86. In certain embodiments, the rAAV genome comprises the nucleic acid sequence of SEQ ID NO: 87.

In certain embodiments, the rAAV genome further comprises a 5′ inverted terminal repeat (5′ ITR) nucleotide sequence 5′ to the first polyadenylation sequence, and a 3′ inverted terminal repeat (3′ ITR) nucleotide sequence 3′ the second polyadenylation sequence.

In certain embodiments, the 5′ ITR nucleotide sequence is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 14, and/or the 3′ ITR nucleotide sequence is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 18.

In certain embodiments, the rAAV genome comprises the nucleic acid sequence of SEQ ID NO: 88. In certain embodiments, the rAAV genome comprises the nucleic acid sequence of SEQ ID NO: 89. In certain embodiments, the rAAV genome comprises the nucleic acid sequence of SEQ ID NO: 90. In certain embodiments, the rAAV genome comprises the nucleic acid sequence of SEQ ID NO: 91.

In another aspect, the disclosure provides a recombinant adeno-associated virus (rAAV) comprising:

(a) an AAV capsid comprising an AAV capsid protein; and
(b) an rAAV genome of any of the above embodiments.

In certain embodiments, the capsid protein is selected from the group consisting of AAV1, AAV2, AAV3, AAV4, AAV5, AAV6, AAV7, AAV8, and AAV9.

In certain embodiments, the AAV capsid protein comprises an amino acid sequence that is at least 95% identical to the amino acid sequence of amino acids 203-736 of SEQ ID NO: 1, 2, 3, 4, 5, 6, 7, 8, 9, 10, 11, 12, 13, 15, 16, or 17.

In certain embodiments, the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G.

In certain embodiments,

(a) the amino acid in the capsid protein corresponding to amino acid 626 of SEQ ID NO: 16 is G, and the amino acid in the capsid protein corresponding to amino acid 718 of SEQ ID NO: 16 is G;
(b) the amino acid in the capsid protein corresponding to amino acid 296 of SEQ ID NO: 16 is H, the amino acid in the capsid protein corresponding to amino acid 464 of SEQ ID NO: 16 is N, the amino acid in the capsid protein corresponding to amino acid 505 of SEQ ID NO: 16 is R, and the amino acid in the capsid protein corresponding to amino acid 681 of SEQ ID NO: 16 is M;
(c) the amino acid in the capsid protein corresponding to amino acid 505 of SEQ ID NO: 16 is R, and the amino acid in the capsid protein corresponding to amino acid 687 of SEQ ID NO: 16 is R;
(d) the amino acid in the capsid protein corresponding to amino acid 346 of SEQ ID NO: 16 is A, and the amino acid in the capsid protein corresponding to amino acid 505 of SEQ ID NO: 16 is R; or
(e) the amino acid in the capsid protein corresponding to amino acid 501 of SEQ ID NO: 16 is I, the amino acid in the capsid protein corresponding to amino acid 505 of SEQ ID NO: 16 is R, and the amino acid in the capsid protein corresponding to amino acid 706 of SEQ ID NO: 16 is C.

In certain embodiments, the capsid protein comprises the amino acid sequence of amino acids 203-736 of SEQ ID NO: 1, 2, 3, 4, 5, 6, 7, 8, 9, 10, 11, 12, 13, 15, 16, or 17.

In certain embodiments, the AAV capsid protein comprises an amino acid sequence that is at least 95% identical to the amino acid sequence of amino acids 138-736 of SEQ ID NO: 1, 2, 3, 4, 5, 6, 7, 8, 9, 10, 11, 12, 13, 15, 16, or 17.

In certain embodiments, the amino acid in the capsid protein corresponding to amino acid 151 of SEQ ID NO: 16 is R; the amino acid in the capsid protein corresponding to amino acid 160 of SEQ ID NO: 16 is D; the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G.

In certain embodiments,

(a) the amino acid in the capsid protein corresponding to amino acid 626 of SEQ ID NO: 16 is G, and the amino acid in the capsid protein corresponding to amino acid 718 of SEQ ID NO: 16 is G;
(b) the amino acid in the capsid protein corresponding to amino acid 296 of SEQ ID NO: 16 is H, the amino acid in the capsid protein corresponding to amino acid 464 of SEQ ID NO: 16 is N, the amino acid in the capsid protein corresponding to amino acid 505 of SEQ ID NO: 16 is R, and the amino acid in the capsid protein corresponding to amino acid 681 of SEQ ID NO: 16 is M;
(c) the amino acid in the capsid protein corresponding to amino acid 505 of SEQ ID NO: 16 is R, and the amino acid in the capsid protein corresponding to amino acid 687 of SEQ ID NO: 16 is R;
(d) the amino acid in the capsid protein corresponding to amino acid 346 of SEQ ID NO: 16 is A, and the amino acid in the capsid protein corresponding to amino acid 505 of SEQ ID NO: 16 is R; or
(e) the amino acid in the capsid protein corresponding to amino acid 501 of SEQ ID NO: 16 is I, the amino acid in the capsid protein corresponding to amino acid 505 of SEQ ID NO: 16 is R, and the amino acid in the capsid protein corresponding to amino acid 706 of SEQ ID NO: 16 is C.

In certain embodiments, the capsid protein comprises the amino acid sequence of amino acids 138-736 of SEQ ID NO: 1, 2, 3, 4, 5, 6, 7, 9, 10, 11, 12, 13, 15, 16, or 17.

In certain embodiments, the AAV capsid protein comprises an amino acid sequence that is at least 95% identical to the amino acid sequence of amino acids 1-736 of SEQ ID NO: 1, 2, 3, 4, 5, 6, 7, 8, 9, 10, 11, 12, 13, 15, 16, or 17.

In certain embodiments, the amino acid in the capsid protein corresponding to amino acid 2 of SEQ ID NO: 16 is T; the amino acid in the capsid protein corresponding to amino acid 65 of SEQ ID NO: 16 is I; the amino acid in the capsid protein corresponding to amino acid 68 of SEQ ID NO: 16 is V; the amino acid in the capsid protein corresponding to amino acid 77 of SEQ ID NO: 16 is R; the amino acid in the capsid protein corresponding to amino acid 119 of SEQ ID NO: 16 is L; the amino acid in the capsid protein corresponding to amino acid 151 of SEQ ID NO: 16 is R; the amino acid in the capsid protein corresponding to amino acid 160 of SEQ ID NO: 16 is D; the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G.

In certain embodiments,

(a) the amino acid in the capsid protein corresponding to amino acid 2 of SEQ ID NO: 16 is T, and the amino acid in the capsid protein corresponding to amino acid 312 of SEQ ID NO: 16 is Q;
(b) the amino acid in the capsid protein corresponding to amino acid 65 of SEQ ID NO: 16 is I, and the amino acid in the capsid protein corresponding to amino acid 626 of SEQ ID NO: 16 is Y;
(c) the amino acid in the capsid protein corresponding to amino acid 77 of SEQ ID NO: 16 is R, and the amino acid in the capsid protein corresponding to amino acid 690 of SEQ ID NO: 16 is K;
(d) the amino acid in the capsid protein corresponding to amino acid 119 of SEQ ID NO: 16 is L, and the amino acid in the capsid protein corresponding to amino acid 468 of SEQ ID NO: 16 is S;
(e) the amino acid in the capsid protein corresponding to amino acid 626 of SEQ ID NO: 16 is G, and the amino acid in the capsid protein corresponding to amino acid 718 of SEQ ID NO: 16 is G;
(f) the amino acid in the capsid protein corresponding to amino acid 296 of SEQ ID NO: 16 is H, the amino acid in the capsid protein corresponding to amino acid 464 of SEQ ID NO: 16 is N, the amino acid in the capsid protein corresponding to amino acid 505 of SEQ ID NO: 16 is R, and the amino acid in the capsid protein corresponding to amino acid 681 of SEQ ID NO: 16 is M;
(g) the amino acid in the capsid protein corresponding to amino acid 505 of SEQ ID NO: 16 is R, and the amino acid in the capsid protein corresponding to amino acid 687 of SEQ ID NO: 16 is R;
(h) the amino acid in the capsid protein corresponding to amino acid 346 of SEQ ID NO: 16 is A, and the amino acid in the capsid protein corresponding to amino acid 505 of SEQ ID NO: 16 is R; or
(i) the amino acid in the capsid protein corresponding to amino acid 501 of SEQ ID NO: 16 is I, the amino acid in the capsid protein corresponding to amino acid 505 of SEQ ID NO: 16 is R, and the amino acid in the capsid protein corresponding to amino acid 706 of SEQ ID NO: 16 is C.

In certain embodiments, the capsid protein comprises the amino acid sequence of amino acids 1-736 of SEQ ID NO: 1, 2, 3, 4, 5, 6, 7, 8, 9, 10, 11, 12, 13, 15, 16, or 17.

In one aspect, the disclosure provides a polynucleotide comprising the nucleic acid sequence set forth in SEQ ID NOs: 85-93.

In one aspect, the disclosure provides a pharmaceutical composition comprising an rAAV described above or the polynucleotide described above.

In one aspect, the disclosure provides a packaging system for preparation of an rAAV, wherein the packaging system comprises:

(a) a first nucleotide sequence encoding one or more AAV Rep proteins;
(b) a second nucleotide sequence encoding a capsid protein of the rAAV described above; and
(c) a third nucleotide sequence comprising an rAAV genome sequence of the rAAV described above.

In certain embodiments, the packaging system comprises a first vector comprising the first nucleotide sequence and the second nucleotide sequence, and a second vector comprising the third nucleotide sequence.

In certain embodiments, the packaging system further comprises a fourth nucleotide sequence comprising one or more helper virus genes. In certain embodiments, the fourth nucleotide sequence is comprised within a third vector. In certain embodiments, the fourth nucleotide sequence comprises one or more genes from a virus selected from the group consisting of adenovirus, herpes virus, vaccinia virus, and cytomegalovirus (CMV).

In certain embodiments, the first vector, second vector, and/or the third vector is a plasmid.

In one aspect, the disclosure provides a method for recombinant preparation of an rAAV, the method comprising introducing the packaging system described above into a cell under conditions whereby the rAAV is produced.

In another aspect, the disclosure provides the rAAV described above, the pharmaceutical composition described above, or the polynucleotide described above, for use as a medicament.

In another aspect, the disclosure provides the rAAV described above, the pharmaceutical composition described above, or the polynucleotide described above, for use in the treatment of complement C5-associated disease.

In another aspect, the disclosure provides the rAAV described above, the pharmaceutical composition described above, or the polynucleotide described above, for use in a method of treating a subject having a complement C5-associated disease, the method comprising administering to the subject an effective amount of the rAAV, the pharmaceutical composition, or the polynucleotide.

In one aspect, the disclosure provides a method of producing an antibody in a subject, the method comprising administering to the subject the pharmaceutical composition of described above.

In certain embodiments, the pharmaceutical composition is administered intravenously.

In one aspect, the disclosure provides a method of treating a complement C5-associated disease in a subject in need thereof, the method comprising administering to the subject an effective amount of the rAAV described above, the pharmaceutical composition described above or the polynucleotide described above.

In certain embodiments, the complement C5-associated disease is selected from the group consisting of geographic atrophy (GA), Guillain-Barré syndrome, myasthenia gravis, systemic lupus erythematous (SLE) nephritis, proliferative nephritis, asthma, rheumatoid arthritis, sepsis, paroxysmal nocturnal hemoglobinuria (PNH), atypical hemolytic uremic syndrome (aHUS), and age-related macular degeneration (AMD).

In certain embodiments, the rAAV, the pharmaceutical composition, or the polynucleotide is administered intravenously.

### BRIEF DESCRIPTION OF THE DRAWINGS

FIG. 1 depicts vector maps of the expression cassettes of rAAV vectors CSAb01, CSAb02, CSAb03 and CSAb04.

FIG. 2A-FIG. 2I depict graphs showing the anti-C5 antibody concentration in the serum of NOD SCID mice receiving anti-C5 antibody expressing vectors (C5Ab02, CSAb03, and CSAb04) packaged in the AAVHSC13, AAVHSC15, or AAVHSC17 capsid. FIG. 2A depicts a graph showing the anti-C5 antibody concentration in the serum of mice receiving vector CSAb04 packaged in the AAVHSC13 or AAVHSC17 capsid at a dose of 1e13 vgs/kg. Data for male and female mice were segregated and multiple serum samples were taken over a period of 23 weeks. FIG. 2B depicts a graph showing the results in FIG. 2A with the Y-axis in a logarithmic scale. FIG. 2C depicts a graph showing the anti-C5 antibody concentration in the serum of mice receiving vector CSAb02 packaged in the AAVHSC17 capsid at a dose of 1e13 vgs/kg. Data for male and female mice were segregated and multiple serum samples were taken over a period of 16 weeks. FIG. 2D depicts a graph showing the results in FIG. 2C with the Y-axis in a logarithmic scale. FIG. 2E depicts a graph showing the anti-C5 antibody concentration in the serum of mice receiving vector CSAb02, CSAb03, or CSAb04, each packaged in the AAVHSC15 or AAVHSC17 capsid at a dose of 1e13 vgs/kg. Data for male mice is shown and multiple serum samples were taken over a period of 16 weeks. FIG. 2F shows the results in FIG. 2E depicted in a line graph format, and FIG. 2G shows the results in FIG. 2E depicted in a line graph format with the Y-axis in a logarithmic scale. FIG. 2H depicts a graph showing the anti-C5 antibody concentration in the serum of mice receiving vector CSAb04 packaged in the AAVHSC17 capsid at 5 doses, Sell vgs/kg, 5e12 vgs/kg, 1.4e13 vgs/kg, 4.4e13 vgs/kg, and 1.8e14 vgs/kg. Data for male mice is shown and multiple serum samples were taken over a period of 13 weeks. FIG. 2I depicts a graph showing the results in FIG. 2H with the Y-axis in a logarithmic scale.

FIG. 3A-FIG. 3C depict graphs showing anti-C5 antibody concentrations in the serum of NOD SCID male mice receiving anti-C5 antibody expressing vectors. The data is derived from FIG. 2 above and ordered to compare vectors C5Ab02, C5Ab03, or C5Ab04 packaged in the AAVHSC13, AAVHSC15, or AAVHSC17 capsid. FIG. 3A depicts a graph showing the anti-C5 antibody concentration in the serum of mice receiving vector C5Ab04 packaged in the AAVHSC13 capsid at a dose of 1e13 vgs/kg. FIG. 3B depicts a graph showing the anti-C5 antibody concentration in the serum of mice receiving vector C5Ab02, C5Ab03, or C5Ab04 packaged in the AAVHSC15 capsid at a dose of 1e13 vgs/kg. FIG. 3C depicts a graph showing the anti-C5 antibody concentration in the serum of mice receiving vector C5Ab02, C5Ab03, or C5Ab04 packaged in the AAVHSC17 capsid at a dose of 1e13 vgs/kg.

FIG. 4A depicts a graph comparing the predicted anti-C5 antibody concentrations in PNH patients receiving chronic maintenance therapy with anti-C5 antibodies eculizumab and ravulizumab to anti-C5 antibody concentrations measured in NOD SCID male and female mice using either the AAVHSC13 or AAVHSC17 capsid (data from FIG. 2B). FIG. 4B depicts a graph comparing the predicted anti-C5 antibody concentrations in PNH patients receiving anti-C5 antibodies eculizumab and ravulizumab to anti-C5 antibody concentrations measured in NOD SCID and HuLiv mice using the AAVHSC17 capsid (representative data from FIGS. 2A, 2I, and 8B).

FIG. 5 depicts a graph of % hemolysis of activated sheep red blood cells (RBCs) at various concentrations of anti-C5 antibody in an ex vivo hemolysis assay. An anti-C5 control antibody was compared against serum obtained from mice treated with AAVHSC13-packaged C5Ab04 and AAVHSC17-packaged C5Ab04.

FIG. 6A depicts a graph of serum antibody concentration and FIG. 6B depicts a graph of % hemolysis of activated sheep RBCs in an ex vivo hemolysis assay. Negative control mouse serum was compared against serum obtained from mice treated with AAVHSC13-packaged C5Ab04 and AAVHSC17-packaged C5Ab04, each at a dose of 1e13 vgs/kg, at 1, 3, 5, 7, and 9 weeks after administration. FIG. 6C depicts a graph showing the results in FIG. 6B with % hemolysis determined from serum samples obtained out to 19 weeks post-administration, and presented in a line graph. FIG. 6D depicts a graph of % hemolysis of activated sheep RBCs in an ex vivo hemolysis assay performed using serum obtained from mice treated with AAVHSC17-packaged C5Ab02 at a dose of 1e13 vgs/kg. FIG. 6E depicts a graph of % hemolysis of activated sheep RBCs in an ex vivo hemolysis assay performed using serum obtained from mice treated with AAVHSC15 or AAVHSC17-packaged C5Ab02, C5Ab03, or C5Ab04, each at a dose of 1e13 vgs/kg. FIG. 6F depicts a graph of % hemolysis of activated sheep RBCs in an ex vivo hemolysis assay performed using serum obtained from mice treated with AAVHSC17-packaged C5Ab04 at doses of Sell vgs/kg, 5e12 vgs/kg, 1.4e13 vgs/kg, 4.4e13 vgs/kg, and 1.8e14 vgs/kg. In FIGS. 6A-6F, data for male and female mice was segregated, and data in FIGS. 6E and 6F were from male mice.

FIG. 7A-FIG. 7B depict graphs comparing: the level of human C5 in the serum of FRGKO humanized liver mice (referred hereafter as HuLiv, Yecuris) in either C57Bl/6 (FRGC57) or NOD (FRGNOD) background to the level of human C5 found in human serum (FIG. 7A); and level of mouse C5 in the serum of HuLiv mice in C57B16 or NOD background (FIG. 7B). FRGC57_Donor A represents the HuLiv mouse with hepatocytes from patient donor A (n=3). FRGC57_Donor B represents the HuLiv mouse with hepatocytes from patient donor B (n=3). NOD Donor A represents the HuLiv mouse from a congenic NOD mouse with hepatocytes from patient donor A (n=9).

FIG. 8A depicts a graph of serum antibody concentration of anti-C5 antibodies in HuLiv mice administered 100 μg of an anti-C5 antibody (biosimilar), or C5Ab04 packaged in the AAVHSC17 capsid at a dose of 1e13 vgs/kg or 1e14 vgs/kg, in each case at 0, 1, 3, and 5 weeks after administration. In FIG. 8A, PB indicates pre-bleed. FIG. 8B shows the data depicted in FIG. 8A with serum antibody concentration determined out to week 11 after administration, presented in a line graph. FIG. 8C depicts a graph of % hemolysis of activated sheep RBCs in an ex vivo hemolysis assay performed using serum obtained from mice administered 100 μg of an anti-C5 antibody (biosimilar), or C5Ab04 packaged in the AAVHSC17 capsid at a dose of 1e13 vgs/kg or 1e14 vgs/kg. FIG. 8D depicts a graph showing the level of mouse C5 detected in serum obtained from mice administered a single dose of 100 μg of an anti-C5 antibody (biosimilar), or C5Ab04 packaged in the AAVHSC17 capsid at a dose of 1e13 vgs/kg or 1e14 vgs/kg. FIG. 9A-FIG. 9B depict a western blot (FIG. 9A) and human IgG ELISA data (FIG. 9B) of the level of human C5 in the culture media of primary human and mouse hepatocytes that were transduced with C5Ab02, C5Ab03, or C5Ab04, packaged in AAVHSC15 or AAVHSC17 capsids.

### DETAILED DESCRIPTION

Provided herein are rAAV genomes and rAAV for the expression of antibodies (e.g., anti-C5 antibodies) in cells (e.g., liver cells), and methods for using the same to treat disorders with the same (e.g., disorders associated with C5 activity (e.g., Paroxysmal Nocturnal Hemoglobinuria)). Also provided are nucleic acids, vectors, packaging systems, and methods for making the rAAV.

### I. DEFINITIONS

As used herein, the terms “recombinant adeno-associated virus” or “rAAV” refers to an AAV comprising a genome lacking functional rep and cap genes.

As used herein, the term “rAAV genome” refers to a nucleic acid molecule (e.g., DNA and/or RNA) comprising the genome sequence of an rAAV. The skilled artisan will appreciate that where an rAAV genome comprises a transgene (e.g., an antibody heavy chain or light chain coding sequence operably linked to a transcriptional regulatory element), the rAAV genome can be in the sense or antisense orientation relative to the direction of transcription of the transgene.

As used herein, the term “AAV capsid protein” refers to an AAV VP1, VP2, or VP3 capsid protein.

As used herein, the “percentage identity” between two nucleotide sequences or between two amino acid sequences is calculated by multiplying the number of matches between the pair of aligned sequences by 100, and dividing by the length of the aligned region, including internal gaps. Identity scoring only counts perfect matches, and does not consider the degree of similarity of amino acids to one another. Note that only internal gaps are included in the length, not gaps at the sequence ends.

As used herein, the term “coding sequence” refers to the portion of a complementary DNA (cDNA) that encodes a polypeptide, starting at the start codon and ending at the stop codon. A gene may have one or more coding sequences due to alternative splicing, alternative translation initiation, and variation within the population. A coding sequence may either be wild-type, silently-altered, or intron-inserted. Exemplary anti-C5 heavy chain coding sequences are set forth in SEQ ID NOs: 52 and 83. An exemplary anti-C5 light chain coding sequence is set forth in SEQ ID NO: 53. A coding sequence may be codon optimized. Codon optimization may be performed to enhance expression of the coding sequence in a desired host cell, such as a human cell. Exemplary codon optimized anti-C5 heavy chain coding sequences are set forth in SEQ ID NOs: 94, 95, 101, 107, 113, 114, and 115. Exemplary codon optimized anti-C5 light chain coding sequence is set forth in SEQ ID NO: 98, 104, 110, or 131.

In certain embodiments, two or more coding sequences (e.g., an antibody heavy chain coding sequence and an antibody light chain coding sequence) can be separated by a nucleotide sequence encoding a peptide cleavage sequence, such as the 2A peptide ribosomal skipping elements. Exemplary 2A peptide cleavage sequences are set forth in SEQ ID NO: 28 or 125 (T2A peptide cleavage sequences), or 128 (P2A peptide cleavage sequence). The 2A peptide cleavage sequences may further comprise a furin cleavage sequence and linker. Exemplary 2A peptide cleavage sequences with the furin cleavage sequence and linker are set forth in SEQ ID NO: 127 or 129.

As used herein, the term “polyadenylation sequence” refers to a DNA sequence that when transcribed into RNA constitutes a polyadenylation signal sequence. The polyadenylation sequence can be native or exogenous. The exogenous polyadenylation sequence can be a mammalian or a viral polyadenylation sequence (e.g., a bovine growth hormone polyadenylation sequence or an SV40 polyadenylation sequence).

As used herein, the term “intron element” refers to a cis-acting nucleotide sequence, for example, a DNA sequence, that regulates (e.g., controls, increases, or reduces) expression of a transgene. In certain embodiments, an intron element is a modified intron, e.g., a synthetic intron sequence. In certain embodiments, an intron element is an exogenous intron element and is derived from an intron exogenous to the transgene it may regulate. In certain embodiments, an intron element comprises a modified splice acceptor and/or splice donor resulting in more robust splicing activity. While not wishing to be bound by theory, it is hypothesized that introns can increase transgene expression, for example, by reducing transcriptional silencing and enhancing mRNA export from the nucleus to the cytoplasm. A skilled worker will appreciate that synthetic intron sequences can be designed to mediate RNA splicing by introducing any consensus splicing motifs known in the art (e.g., in Sibley et al. (2016) Nature Reviews Genetics, 17, 407-21, which is incorporated by reference herein in its entirety). Exemplary intron sequences are provided in Lu et al. (2013) Molecular Therapy 21(5): 954-63, and Lu et al. (2017) Hum. Gene Ther. 28(1): 125-34, which are incorporated by reference herein in their entirety.

As used herein, the term “silently-altered” refers to alteration of a coding sequence of a gene (e.g., by nucleotide substitution) without changing the amino acid sequence of the polypeptide encoded by the coding sequence or stuffer-inserted coding sequence. Such silent alteration is advantageous in that it may increase the translation efficiency of a coding sequence, and/or prevent recombination with a corresponding sequence of an endogenous gene when a coding sequence is transduced into a cell.

As used herein, the term “transcriptional regulatory element” or “TRE” refers to a cis-acting nucleotide sequence, for example, a DNA sequence, that regulates (e.g., controls, increases, or reduces) transcription of an operably linked nucleotide sequence by an RNA polymerase to form an RNA molecule. A TRE relies on one or more trans-acting molecules, such as transcription factors, to regulate transcription. Thus, one TRE may regulate transcription in different ways when it is in contact with different trans-acting molecules, for example, when it is in different types of cells. A TRE may comprise one or more promoter elements and/or enhancer elements. A skilled artisan would appreciate that the promoter and enhancer elements in a gene may be close in location, and the term “promoter” may refer to a sequence comprising a promoter element and an enhancer element. Thus, the term “promoter” does not exclude an enhancer element in the sequence. The promoter and enhancer elements do not need to be derived from the same gene or species, and the sequence of each promoter or enhancer element may be either identical or substantially identical to the corresponding endogenous sequence in the genome.

As used herein, the term “operably linked” is used to describe the connection between a TRE and a coding sequence to be transcribed. Typically, gene expression is placed under the control of a TRE comprising one or more promoter and/or enhancer elements. The coding sequence is “operably linked” to the TRE if the transcription of the coding sequence is controlled or influenced by the TRE. The promoter and enhancer elements of the TRE may be in any orientation and/or distance from the coding sequence, as long as the desired transcriptional activity is obtained. In certain embodiments, the TRE is upstream from the coding sequence.

In the instant disclosure, nucleotide positions in an antibody coding sequence (e.g., an antibody heavy chain coding sequence or an antibody light chain coding sequence) are specified relative to the first nucleotide of the start codon. The first nucleotide of a start codon is position 1; the nucleotides 5′ to the first nucleotide of the start codon have negative numbers; the nucleotides 3′ to the first nucleotide of the start codon have positive numbers.

As used herein, the term “expression cassette” refers to a polynucleotide sequence comprising, from 5′ to 3′, a transcriptional regulatory element (TRE), a coding sequence encoding a polypeptide, and a polyadenylation sequence. In certain embodiments, an intron is present between the TRE and the coding sequence. In certain embodiments, the coding sequence encodes an antibody heavy chain or an antibody light chain.

As used herein, the term “effective amount” in the context of the administration of an AAV to a subject refers to the amount of the AAV that achieves a desired prophylactic or therapeutic effect.

As used herein, the term “about” or “approximately” when referring to a measurable value, such as the expression level of an antibody (e.g., an antibody heavy chain and antibody light chain), encompasses variations of ±20% or ±10%, ±5%, ±1%, or ±0.1% of a given value or range, as are appropriate to perform the methods disclosed herein.

### II. ADENO-ASSOCIATED VIRUS COMPOSITIONS

In one aspect, provided herein are novel rAAV genomes comprising a transcriptional regulatory element (TRE) operably linked to at least a portion of an antibody coding sequence (e.g., an anti-C5 antibody heavy chain coding sequence and/or anti-C5 antibody light chain coding sequence). The rAAV genomes provided herein are useful for extrachromosomal expression of an antibody in a cell comprising the rAAV genome.

The rAAV genome can be used to express antibodies in any mammalian cells (e.g., human cells). Thus, the TRE can be active in any mammalian cells (e.g., human cells). In certain embodiments, the TRE is active in a broad range of human cells. Such TREs may comprise constitutive promoter and/or enhancer elements including cytomegalovirus (CMV) promoter/enhancer (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 54, 55, or 56), SV40 promoter, chicken ACTB promoter (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 47 or 57), JeT promoter (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 58), smCBA promoter (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 59), human elongation factor 1 alpha (EF1α) promoter (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 39), minute virus of mouse (MVM) intron which comprises transcription factor binding sites (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 30 or 61), human phosphoglycerate kinase (PGK1) promoter, human ubiquitin C (Ubc) promoter, human beta actin promoter, human neuron-specific enolase (ENO2) promoter, human beta-glucuronidase (GUSB) promoter, a rabbit beta-globin element (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 41), human calmodulin 1 (CALM1) promoter (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 44), and/or human Methyl-CpG Binding Protein 2 (MeCP2) promoter. Any of these TREs can be combined in any order to drive efficient transcription. For example, an rAAV genome may comprise a CMV enhancer, a CBA promoter, and the splice acceptor from exon 3 of the rabbit beta-globin gene, collectively called a CAG promoter (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 42). For example, an rAAV genome may comprise a hybrid of CMV enhancer and CBA promoter followed by a splice donor and splice acceptor, collectively called a CASI promoter region (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 48 or 65).

Alternatively, the TRE may be a tissue-specific TRE, i.e., it is active in specific tissue(s) and/or organ(s). A tissue-specific TRE comprises one or more tissue-specific promoter and/or enhancer elements, and optionally one or more constitutive promoter and/or enhancer elements. A skilled artisan would appreciate that tissue-specific promoter and/or enhancer elements can be isolated from genes specifically expressed in the tissue by methods well known in the art.

In certain embodiments, the TRE is liver-specific (e.g., hepatocyte-specific). Exemplary liver-specific TREs may comprise one or more elements selected from the group consisting of human albumin promoter, human transthyretin (TTR) promoter (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 66), human APOE/C-I hepatic control region (HCR) 1 (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 25 or 68), human APOH promoter, and human SERPINA1 (hAAT) promoter (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 26, 69 or 70) or a hepatic specific regulatory module thereof (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 71). In certain embodiments, an hAAT promoter region comprises a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 72. In certain embodiments, the liver-specific TRE comprises the TBG SERPINA7 promoter as described in Yan et al. (Gene (2016) 506, 289-294) (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 116). In certain embodiments, the liver-specific TRE comprises the TBG SERPINA7 promoter as described in Hayashi et al. (Molecular Endocrinology (1993) 7(8), 1049-1060) (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 117). In certain embodiments, the liver-specific TRE comprises the hAAT SERPINA1 promoter as described in Hafenrichter et al. (Blood (1994) 84(10), 3394-3404) (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 118). In certain embodiments, the liver-specific TRE comprises the TTR promoter as described in Costa et al. (Molecular and Cellular Biology (1988) 8(1), 81-90) (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 119). In certain embodiments, the liver-specific TRE comprises the ApoA2 promoter as described in Kan et al. (Nucleic Acids Research (1999) 27(4), 1104-1117) (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 120). In certain embodiments, the liver-specific TRE comprises the albumin promoter as described in Tang et al. (Biomedical Reports (2017) 6, 627-632) (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 121). In certain embodiments, the liver-specific TRE comprises the modified fibrinogen promoter as described in Kyostio-Moore et al. (Molecular Therapy (2016) 3, 16006) (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 122). In certain embodiments, the liver-specific TRE comprises the minimum human APOE/C-I hepatic control region (HCR) 1 promoter as described in Dang et al. (J. Biol. Chem. (1995) 270(38), 22557-85) (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 123). In certain embodiments, the liver-specific TRE comprises the human APOE/C-I hepatic control region (HCR) 2 promoter as described in Allan et al. (J. Biol. Chem. (1995) 270(44), 26278-81) (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 124). More liver-specific promoter elements are disclosed in WO 2009/130208 and Kramer et al. (Molecular Therapy (2003) 7, 375-385), which are incorporated by reference herein in their entirety.

In certain embodiments, the rAAV genome comprises two or more TREs, optionally comprising at least one of the TREs disclosed above. A skilled person in the art would appreciate that any of these TREs can be combined in any order, and combinations of a constitutive TRE and a tissue-specific TRE can drive efficient and tissue-specific transcription. For example, in certain embodiments, the rAAV genome comprises a human HCR1 (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 25, 68, or 123) and a human EF-1α promoter (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 39), optionally wherein the human HCR1 is 5′ to the human EF-la promoter. In certain embodiments, the rAAV genome comprises a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the sequence nucleotide set forth in SEQ ID NO: 60.

Similarly, combinations of two or more tissue-specific TREs can drive efficient and tissue-specific transcription. For example, in certain embodiments, the rAAV genome comprises a human HCR1 (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 25, 68, or 123) and a hAAT promoter (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 26), optionally wherein the human HCR1 is 5′ to the hAAT promoter. In certain embodiments, the rAAV genome comprises a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 27. In certain embodiments, the rAAV genome comprises a human HCR1 (e.g., comprising the nucleotide sequence set forth in SEQ ID NO: 25) and a hAAT promoter (e.g., comprising the nucleotide sequence set forth in SEQ ID NO: 26), optionally wherein the human HCR1 is 5′ to the hAAT promoter. In certain embodiments, the rAAV genome comprises the nucleotide sequence set forth in SEQ ID NO: 27.

In certain embodiments, the rAAV genome comprises a hepatic specific regulatory module of hAAT promoter (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 71) and a human TTR promoter (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 66), optionally wherein the hepatic specific regulatory module is 5′ to the human TTR promoter. In certain embodiments, the rAAV genome comprises a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 67. In certain embodiments, the rAAV genome comprises a hepatic specific regulatory module of hAAT promoter (e.g., comprising the nucleotide sequence set forth in SEQ ID NO: 71) and a human TTR promoter (e.g., comprising the nucleotide sequence set forth in SEQ ID NO: 66), optionally wherein the hepatic specific regulatory module is 5′ to the human TTR promoter. In certain embodiments, the rAAV genome comprises the nucleotide sequence set forth in SEQ ID NO: 67.

In certain embodiments, the rAAV genome further comprises an intron element 5′ to the at least a portion of an antibody coding sequence. Such intron elements can increase transgene expression, for example, by reducing transcriptional silencing and enhancing mRNA export from the nucleus to the cytoplasm. In certain embodiments, the rAAV genome comprises from 5′ to 3′: a TRE, an intron element, and the at least a portion of an antibody coding sequence.

The intron element can comprise at least a portion of a native intron sequence of an immunoglobulin gene, or the intron element can be an exogenous intron element (e.g., comprising at least an intron sequence from a different species or a different gene from the same species, and/or a synthetic intron sequence). In certain embodiments, the intron element is an exogenous intron element comprising at least a portion of an intron sequence from a different species. In certain embodiments, the intron element is an exogenous intron element comprising at least a portion of an intron sequence from a different gene from the same species. In certain embodiments, the intron element is an exogenous intron element comprising a synthetic intron sequence. In certain embodiments, the intron element is an exogenous intron element comprising a combination of at least an intron sequence from a different species or a different gene from the same species, and/or a synthetic intron sequence.

A skilled worker will appreciate that intron elements can be designed to mediate RNA splicing by introducing any consensus splicing motifs known in the art (e.g., in Sibley et al., (2016) Nature Reviews Genetics, 17, 407-21, which is incorporated by reference herein in its entirety). Exemplary intron sequences are provided in Lu et al. (2013) Molecular Therapy 21(5): 954-63, and Lu et al. (2017) Hum. Gene Ther. 28(1): 125-34, which are incorporated by reference herein in their entirety.

In certain embodiments, the rAAV genome comprises an exogenous intron element. In certain embodiments, the rAAV comprises an SV40 intron element (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 29), a minute virus of mouse (MVM) intron (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 30 or 61), or a synthetic intron (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 45). In certain embodiments, the rAAV genome comprises an SV40 intron element (e.g., comprising the nucleotide sequence set forth in SEQ ID NO: 29), a minute virus of mouse (MVM) intron element (e.g., comprising the nucleotide sequence set forth in SEQ ID NOs: 30 and 61), or a synthetic intron (e.g., comprising a nucleotide sequence set forth in SEQ ID NO: 45).

In certain embodiments, the rAAV genome comprises from 5′ to 3′: a TRE and an intron element. In certain embodiments, the combined TRE and intron element has at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 43 or 50. In certain embodiments, the combined TRE and intron element comprises a nucleotide sequence of SEQ ID NO: 43 or 50. In certain embodiments, the combined TRE and intron element consists of a nucleotide sequence of SEQ ID NO: 43 or 50.

In certain embodiments, the rAAV genome disclosed herein further comprises a transcription terminator (e.g., a polyadenylation sequence). In certain embodiments, the transcription terminator is 3′ to the at least a portion of an antibody coding sequence. The transcription terminator may be any sequence that effectively terminates transcription, and a skilled artisan would appreciate that such sequences can be isolated from any genes that are expressed in the cell in which transcription of the at least a portion of an antibody coding sequence is desired. In certain embodiments, the transcription terminator comprises a polyadenylation sequence. In certain embodiments, the polyadenylation sequence is identical or substantially identical to the endogenous polyadenylation sequence of an immunoglobulin gene. In certain embodiments, the polyadenylation sequence is an exogenous polyadenylation sequence. In certain embodiments, the polyadenylation sequence is an SV40 polyadenylation sequence (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 31, 34, or 35, or a nucleotide sequence complementary thereto). In certain embodiments, the polyadenylation sequence comprises the nucleotide sequence set forth in SEQ ID NO: 31. In certain embodiments, the polyadenylation sequence consists of the nucleotide sequence set forth in SEQ ID NO: 31. In certain embodiments, the polyadenylation sequence is a bovine growth hormone (BGH) polyadenylation sequence (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 33, or a nucleotide sequence complementary thereto). In certain embodiments, the polyadenylation sequence comprises the nucleotide sequence set forth in SEQ ID NO: 32. In certain embodiments, the polyadenylation sequence consists of the nucleotide sequence set forth in SEQ ID NO: 32.

In certain embodiments, the rAAV genome comprises from 5′ to 3′: a TRE, an intron element, at least a portion of an antibody coding sequence, and a polyadenylation sequence. In certain embodiments, the TRE has at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ ID NOs: 25-27, 36, 39, 42, 44, 46-49, 54-60, or 65-72; the intron element has at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 29, 30, or 61; the at least a portion of an antibody coding sequence has at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NOs: 52, 53, 62, 63, 83, 94, 95, 97, 98, 100, 101, 103, 104, 106, 107, 109, 110, 112, 113, 114, 115, 131, and 132; and/or the polyadenylation sequence has at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ ID NOs: 31, 33, 34, or 35.

In certain embodiments, the TRE comprises a nucleotide sequence selected from the group consisting of SEQ ID NOs: 25-27, 36, 39, 42, 44, 46-49, 54-60, and 65-72; the intron element comprises a nucleotide sequence selected from the group consisting of SEQ ID NOs: 29, 30, and 61; the at least a portion of an antibody coding sequence comprises the nucleotide sequence set forth in SEQ ID NO: 52; and/or the polyadenylation sequence comprises a nucleotide sequence selected from the group consisting of SEQ ID NOs: 31, 33, 34, and 35.

In certain embodiments, the TRE comprises a nucleotide sequence selected from the group consisting of SEQ ID NOs: 25-27, 36, 39, 42, 44, 46-49, 54-60, and 65-72; the intron element comprises a nucleotide sequence selected from the group consisting of SEQ ID NOs: 29, 30, and 61; the at least a portion of an antibody coding sequence comprises the nucleotide sequence set forth in SEQ ID NO: 53; and/or the polyadenylation sequence comprises a nucleotide sequence selected from the group consisting of SEQ ID NOs: 31, 33, 34, and 35.

In certain embodiments, the TRE comprises a nucleotide sequence selected from the group consisting of SEQ ID NOs: 25-27, 36, 39, 42, 44, 46-49, 54-60, and 65-72; the intron element comprises a nucleotide sequence selected from the group consisting of SEQ ID NOs: 29, 30, and 61; the at least a portion of an antibody coding sequence comprises the nucleotide sequence set forth in SEQ ID NO: 62; and/or the polyadenylation sequence comprises a nucleotide sequence selected from the group consisting of SEQ ID NOs: 31, 33, 34, and 35.

In certain embodiments, the TRE comprises a nucleotide sequence selected from the group consisting of SEQ ID NOs: 25-27, 36, 39, 42, 44, 46-49, 54-60, and 65-72; the intron element comprises a nucleotide sequence selected from the group consisting of SEQ ID NOs: 29, 30, and 61; the at least a portion of an antibody coding sequence comprises the nucleotide sequence set forth in SEQ ID NO: 63; and/or the polyadenylation sequence comprises a nucleotide sequence selected from the group consisting of SEQ ID NOs: 31, 33, 34, and 35.

In certain embodiments, the TRE comprises a nucleotide sequence selected from the group consisting of SEQ ID NOs: 25-27, 36, 39, 42, 44, 46-49, 54-60, and 65-72; the intron element comprises a nucleotide sequence selected from the group consisting of SEQ ID NOs: 29, 30, and 61; the at least a portion of an antibody coding sequence comprises the nucleotide sequence set forth in SEQ ID NO: 83; and/or the polyadenylation sequence comprises a nucleotide sequence selected from the group consisting of SEQ ID NOs: 31, 33, 34, and 35.

In certain embodiments, the TRE comprises or consists of the nucleotide sequence set forth in SEQ ID NO: 25, 26, or 27; the intron element comprises or consists of the nucleotide sequence set forth in SEQ ID NO: 29; the at least a portion of an antibody coding sequence comprises or consists of the nucleotide sequence set forth in SEQ ID NO: 52; and/or the polyadenylation sequence comprises or consists of the nucleotide sequence set forth in SEQ ID NO: 33.

In certain embodiments, the TRE comprises or consists of the nucleotide sequence set forth in SEQ ID NO: 25, 26, or 27; the intron element comprises or consists of the nucleotide sequence set forth in SEQ ID NO: 29; the at least a portion of an antibody coding sequence comprises or consists of the nucleotide sequence set forth in SEQ ID NO: 62; and/or the polyadenylation sequence comprises or consists of the nucleotide sequence set forth in SEQ ID NO: 33.

In certain embodiments, the TRE comprises or consists of the nucleotide sequence set forth in SEQ ID NO: 25, 26, or 27; the intron element comprises or consists of the nucleotide sequence set forth in SEQ ID NO: 29; the at least a portion of an antibody coding sequence comprises or consists of the nucleotide sequence set forth in SEQ ID NO: 83; and/or the polyadenylation sequence comprises or consists of the nucleotide sequence set forth in SEQ ID NO: 33.

In certain embodiments, the TRE comprises or consists of the nucleotide sequence set forth in SEQ ID NO: 66, 67, or 71; the intron element comprises or consists of the nucleotide sequence set forth in SEQ ID NO: 30 or 61; the at least a portion of an antibody coding sequence comprises or consists of the nucleotide sequence set forth in SEQ ID NO: 53; and/or the polyadenylation sequence comprises or consists of the nucleotide sequence set forth in SEQ ID NO: 31.

In certain embodiments, the TRE comprises or consists of the nucleotide sequence set forth in SEQ ID NO: 66, 67, or 71; the intron element comprises or consists of the nucleotide sequence set forth in SEQ ID NO: 30 or 61; the at least a portion of an antibody coding sequence comprises or consists of the nucleotide sequence set forth in SEQ ID NO: 63; and/or the polyadenylation sequence comprises or consists of the nucleotide sequence set forth in SEQ ID NO: 31.

In certain embodiments, the rAAV genome comprises a nucleotide sequence at least 80% (e.g., at least 80%, 81%, 82%, 83%, 84%, 85%, 86%, 87%, 88%, 89%, 90%, 91% 92%, 93%, 94%, 95%, 96%, 97%, 98%, 99%, or 99.5%) identical to any one of SEQ ID NO: 88, 89, 90, or 91. In certain embodiments, the rAAV genome comprises the nucleotide sequence set forth in any one of SEQ ID NO: 88, 89, 90, or 91. In certain embodiments, the rAAV genome consists of the nucleotide sequence set forth in any one of SEQ ID NO: 88, 89, 90, or 91.

In certain embodiments, the rAAV genome comprises a nucleotide sequence at least 80% (e.g., at least 80%, 81%, 82%, 83%, 84%, 85%, 86%, 87%, 88%, 89%, 90%, 91% 92%, 93%, 94%, 95%, 96%, 97%, 98%, 99%, or 99.5%) identical to SEQ ID NO: 88. In certain embodiments, the rAAV genome comprises the nucleotide sequence set forth in SEQ ID NO: 88. In certain embodiments, the rAAV genome consists of the nucleotide sequence set forth in SEQ ID NO: 88.

In certain embodiments, the rAAV genome comprises a nucleotide sequence at least 80% (e.g., at least 80%, 81%, 82%, 83%, 84%, 85%, 86%, 87%, 88%, 89%, 90%, 91% 92%, 93%, 94%, 95%, 96%, 97%, 98%, 99%, or 99.5%) identical to SEQ ID NO: 89. In certain embodiments, the rAAV genome comprises the nucleotide sequence set forth in SEQ ID NO: 89. In certain embodiments, the rAAV genome consists of the nucleotide sequence set forth in SEQ ID NO: 89.

In certain embodiments, the rAAV genome comprises a nucleotide sequence at least 80% (e.g., at least 80%, 81%, 82%, 83%, 84%, 85%, 86%, 87%, 88%, 89%, 90%, 91% 92%, 93%, 94%, 95%, 96%, 97%, 98%, 99%, or 99.5%) identical to SEQ ID NO: 90. In certain embodiments, the rAAV genome comprises the nucleotide sequence set forth in SEQ ID NO: 90. In certain embodiments, the rAAV genome consists of the nucleotide sequence set forth in SEQ ID NO: 90.

In certain embodiments, the rAAV genome comprises a nucleotide sequence at least 80% (e.g., at least 80%, 81%, 82%, 83%, 84%, 85%, 86%, 87%, 88%, 89%, 90%, 91% 92%, 93%, 94%, 95%, 96%, 97%, 98%, 99%, or 99.5%) identical to SEQ ID NO: 91. In certain embodiments, the rAAV genome comprises the nucleotide sequence set forth in SEQ ID NO: 91. In certain embodiments, the rAAV genome consists of the nucleotide sequence set forth in SEQ ID NO: 91.

In certain embodiments, the rAAV genomes disclosed herein further comprise a 5′ inverted terminal repeat (5′ ITR) nucleotide sequence 5′ to the TRE, and a 3′ inverted terminal repeat (3′ ITR) nucleotide sequence 3′ to the polyadenylation sequence associated with an antibody light chain coding sequence. ITR sequences from any AAV serotype or variant thereof can be used in the rAAV genomes disclosed herein. The 5′ and 3′ ITR can be from an AAV of the same serotype or from AAVs of different serotypes. Exemplary ITRs for use in the rAAV genomes disclosed herein are set forth in SEQ ID NOs: 14, 18, 19, 20, 21, and 32, herein.

In certain embodiments, the 5′ ITR or 3′ ITR is from AAV2. In certain embodiments, both the 5′ ITR and the 3′ ITR are from AAV2. In certain embodiments, the 5′ ITR nucleotide sequence has at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 14, or the 3′ ITR nucleotide sequence has at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 18. In certain embodiments, the 5′ ITR nucleotide sequence has at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 14, and the 3′ ITR nucleotide sequence has at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 18. In certain embodiments, the rAAV genome comprises a nucleotide sequence set forth in SEQ ID NO: 43, a 5′ ITR nucleotide sequence having the sequence of SEQ ID NO: 14, and a 3′ ITR nucleotide sequence having the sequence of SEQ ID NO: 18.

In certain embodiments, the 5′ ITR or 3′ ITR are from AAV5. In certain embodiments, both the 5′ ITR and 3′ ITR are from AAV5. In certain embodiments, the 5′ ITR nucleotide sequence has at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 20, or the 3′ ITR nucleotide sequence has at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 21. In certain embodiments, the 5′ ITR nucleotide sequence has at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 20, and the 3′ ITR nucleotide sequence has at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 21. In certain embodiments, the rAAV genome comprises a nucleotide sequence set forth in any one of SEQ ID NO: 43, a 5′ ITR nucleotide sequence having the sequence of SEQ ID NO: 20, and a 3′ ITR nucleotide sequence having the sequence of SEQ ID NO: 21.

In certain embodiments, the 5′ ITR nucleotide sequence and the 3′ ITR nucleotide sequence are substantially complementary to each other (e.g., are complementary to each other except for mismatch at 1, 2, 3, 4, or 5 nucleotide positions in the 5′ or 3′ ITR).

In certain embodiments, the 5′ ITR or the 3′ ITR is modified to reduce or abolish resolution by Rep protein (“non-resolvable ITR”). In certain embodiments, the non-resolvable ITR comprises an insertion, deletion, or substitution in the nucleotide sequence of the terminal resolution site. Such modification allows formation of a self-complementary, double-stranded DNA genome of the AAV after the rAAV genome is replicated in an infected cell. Exemplary non-resolvable ITR sequences are known in the art (see e.g., those provided in U.S. Pat. Nos. 7,790,154 and 9,783,824, which are incorporated by reference herein in their entirety). In certain embodiments, the 5′ ITR comprises a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 19. In certain embodiments, the 5′ ITR consists of a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 19. In certain embodiments, the 5′ ITR consists of the nucleotide sequence set forth in SEQ ID NO: 19. In certain embodiments, the 3′ ITR comprises a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 32. In certain embodiments, the 5′ ITR consists of a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 32. In certain embodiments, the 3′ ITR consists of the nucleotide sequence set forth in SEQ ID NO: 32. In certain embodiments, the 5′ ITR consists of the nucleotide sequence set forth in SEQ ID NO: 19, and the 3′ ITR consists of the nucleotide sequence set forth in SEQ ID NO: 32. In certain embodiments, the 5′ ITR consists of the nucleotide sequence set forth in SEQ ID NO: 19, and the 3′ ITR consists of the nucleotide sequence set forth in SEQ ID NO: 32.

In certain embodiments, the 5′ ITR is flanked by an additional nucleotide sequence derived from a wild-type AAV2 genomic sequence. In certain embodiments, the 5′ ITR is flanked by an additional 46 bp sequence derived from a wild-type AAV2 sequence that is adjacent to a wild-type AAV2 ITR in an AAV2 genome. In certain embodiments, the additional 46 bp sequence is 3′ to the 5′ ITR in the rAAV genome. In certain embodiments, the 46 bp sequence consists of the nucleotide sequence set forth in SEQ ID NO: 74.

In certain embodiments, the 3′ ITR is flanked by an additional nucleotide sequence derived from a wild-type AAV2 genomic sequence. In certain embodiments, the 3′ ITR is flanked by an additional 37 bp sequence derived from a wild-type AAV2 sequence that is adjacent to a wild-type AAV2 ITR in an AAV2 genome. See, e.g., Savy et al., Human Gene Therapy Methods (2017) 28(5): 277-289 (which is hereby incorporated by reference herein in its entirety). In certain embodiments, the additional 37 bp sequence is 5′ to the 3′ ITR in the rAAV genome. In certain embodiments, the 37 bp sequence consists of the nucleotide sequence set forth in SEQ ID NO: 73.

In another aspect, provided herein is a polynucleotide comprising a nucleic acid sequence that is at least 80% (e.g., at least 80%, 81%, 82%, 83%, 84%, 85%, 86%, 87%, 88%, 89%, 90%, 91% 92%, 93%, 94%, 95%, 96%, 97%, 98%, or 99%) identical to the nucleic acid sequence set forth in SEQ ID NO: 84, 85, 86, or 87. In certain embodiments, the polynucleotide comprises or consists of the nucleic acid sequence set forth in SEQ ID NO: 84, 85, 86, or 87.

In another aspect, provided herein are novel rAAV compositions comprising an AAV capsid comprising an AAV capsid protein, an rAAV genome as disclosed herein (e.g., an rAAV genome comprising a transcriptional regulatory element operably linked to an antibody coding sequence (e.g., an antibody heavy chain or light chain coding sequence), allowing for extrachromosomal expression of an antibody in a cell transduced with the AAV).

A capsid protein from any AAV capsid known the art can be used in the rAAV compositions disclosed herein, including, without limitation, a capsid protein from an AAV1, AAV2, AAV3, AAV4, AAV5, AAV6, AAV7, AAV8, or AAV9 serotype. For example, in certain embodiments, the capsid protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence of amino acids 203-736 of SEQ ID NO: 1, 2, 3, 4, 5, 6, 7, 8, 9, 10, 11, 12, 13, 15, 16, or 17. In certain embodiments, the capsid protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence of amino acids 203-736 of SEQ ID NO: 1, 2, 3, 4, 5, 6, 7, 8, 9, 10, 11, 12, 13, 15, 16, or 17, wherein: the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G. In certain embodiments, the amino acid in the capsid protein corresponding to amino acid 626 of SEQ ID NO: 16 is G, and the amino acid in the capsid protein corresponding to amino acid 718 of SEQ ID NO: 16 is G. In certain embodiments, the amino acid in the capsid protein corresponding to amino acid 296 of SEQ ID NO: 16 is H, the amino acid in the capsid protein corresponding to amino acid 464 of SEQ ID NO: 16 is N, the amino acid in the capsid protein corresponding to amino acid 505 of SEQ ID NO: 16 is R, and the amino acid in the capsid protein corresponding to amino acid 681 of SEQ ID NO: 16 is M. In certain embodiments, the amino acid in the capsid protein corresponding to amino acid 505 of SEQ ID NO: 16 is R, and the amino acid in the capsid protein corresponding to amino acid 687 of SEQ ID NO: 16 is R. In certain embodiments, the amino acid in the capsid protein corresponding to amino acid 346 of SEQ ID NO: 16 is A, and the amino acid in the capsid protein corresponding to amino acid 505 of SEQ ID NO: 16 is R. In certain embodiments, the amino acid in the capsid protein corresponding to amino acid 501 of SEQ ID NO: 16 is I, the amino acid in the capsid protein corresponding to amino acid 505 of SEQ ID NO: 16 is R, and the amino acid in the capsid protein corresponding to amino acid 706 of SEQ ID NO: 16 is C. In certain embodiments, the capsid protein comprises the amino acid sequence of amino acids 203-736 of SEQ ID NO: 1, 2, 3, 4, 5, 6, 7, 8, 9, 10, 11, 12, 13, 15, 16, or 17.

For example, in certain embodiments, the capsid protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence of amino acids 138-736 of SEQ ID NO: 1, 2, 3, 4, 5, 6, 7, 8, 9, 10, 11, 12, 13, 15, 16, or 17. In certain embodiments, the capsid protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence of amino acids 138-736 of SEQ ID NO: 1, 2, 3, 4, 5, 6, 7, 8, 9, 10, 11, 12, 13, 15, 16, or 17, wherein: the amino acid in the capsid protein corresponding to amino acid 151 of SEQ ID NO: 16 is R; the amino acid in the capsid protein corresponding to amino acid 160 of SEQ ID NO: 16 is D; the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G. In certain embodiments, the amino acid in the capsid protein corresponding to amino acid 626 of SEQ ID NO: 16 is G, and the amino acid in the capsid protein corresponding to amino acid 718 of SEQ ID NO: 16 is G. In certain embodiments, the amino acid in the capsid protein corresponding to amino acid 296 of SEQ ID NO: 16 is H, the amino acid in the capsid protein corresponding to amino acid 464 of SEQ ID NO: 16 is N, the amino acid in the capsid protein corresponding to amino acid 505 of SEQ ID NO: 16 is R, and the amino acid in the capsid protein corresponding to amino acid 681 of SEQ ID NO: 16 is M. In certain embodiments, the amino acid in the capsid protein corresponding to amino acid 505 of SEQ ID NO: 16 is R, and the amino acid in the capsid protein corresponding to amino acid 687 of SEQ ID NO: 16 is R. In certain embodiments, the amino acid in the capsid protein corresponding to amino acid 346 of SEQ ID NO: 16 is A, and the amino acid in the capsid protein corresponding to amino acid 505 of SEQ ID NO: 16 is R. In certain embodiments, the amino acid in the capsid protein corresponding to amino acid 501 of SEQ ID NO: 16 is I, the amino acid in the capsid protein corresponding to amino acid 505 of SEQ ID NO: 16 is R, and the amino acid in the capsid protein corresponding to amino acid 706 of SEQ ID NO: 16 is C. In certain embodiments, the capsid protein comprises the amino acid sequence of amino acids 138-736 of SEQ ID NO: 1, 2, 3, 4, 5, 6, 7, 8, 9, 10, 11, 12, 13, 15, 16, or 17.

For example, in certain embodiments, the capsid protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence of amino acids 1-736 of SEQ ID NO: 1, 2, 3, 4, 5, 6, 7, 8, 9, 10, 11, 12, 13, 15, 16, or 17. In certain embodiments, the capsid protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence of amino acids 1-736 of SEQ ID NO: 1, 2, 3, 4, 5, 6, 7, 8, 9, 10, 11, 12, 13, 15, 16, or 17, wherein: the amino acid in the capsid protein corresponding to amino acid 2 of SEQ ID NO: 16 is T; the amino acid in the capsid protein corresponding to amino acid 65 of SEQ ID NO: 16 is I; the amino acid in the capsid protein corresponding to amino acid 68 of SEQ ID NO: 16 is V; the amino acid in the capsid protein corresponding to amino acid 77 of SEQ ID NO: 16 is R; the amino acid in the capsid protein corresponding to amino acid 119 of SEQ ID NO: 16 is L; the amino acid in the capsid protein corresponding to amino acid 151 of SEQ ID NO: 16 is R; the amino acid in the capsid protein corresponding to amino acid 160 of SEQ ID NO: 16 is D; the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G. In certain embodiments, the amino acid in the capsid protein corresponding to amino acid 2 of SEQ ID NO: 16 is T, and the amino acid in the capsid protein corresponding to amino acid 312 of SEQ ID NO: 16 is Q. In certain embodiments, the amino acid in the capsid protein corresponding to amino acid 65 of SEQ ID NO: 16 is I, and the amino acid in the capsid protein corresponding to amino acid 626 of SEQ ID NO: 16 is Y. In certain embodiments, the amino acid in the capsid protein corresponding to amino acid 77 of SEQ ID NO: 16 is R, and the amino acid in the capsid protein corresponding to amino acid 690 of SEQ ID NO: 16 is K. In certain embodiments, the amino acid in the capsid protein corresponding to amino acid 119 of SEQ ID NO: 16 is L, and the amino acid in the capsid protein corresponding to amino acid 468 of SEQ ID NO: 16 is S. In certain embodiments, the amino acid in the capsid protein corresponding to amino acid 626 of SEQ ID NO: 16 is G, and the amino acid in the capsid protein corresponding to amino acid 718 of SEQ ID NO: 16 is G. In certain embodiments, the amino acid in the capsid protein corresponding to amino acid 296 of SEQ ID NO: 16 is H, the amino acid in the capsid protein corresponding to amino acid 464 of SEQ ID NO: 16 is N, the amino acid in the capsid protein corresponding to amino acid 505 of SEQ ID NO: 16 is R, and the amino acid in the capsid protein corresponding to amino acid 681 of SEQ ID NO: 16 is M. In certain embodiments, the amino acid in the capsid protein corresponding to amino acid 505 of SEQ ID NO: 16 is R, and the amino acid in the capsid protein corresponding to amino acid 687 of SEQ ID NO: 16 is R. In certain embodiments, the amino acid in the capsid protein corresponding to amino acid 346 of SEQ ID NO: 16 is A, and the amino acid in the capsid protein corresponding to amino acid 505 of SEQ ID NO: 16 is R. In certain embodiments, the amino acid in the capsid protein corresponding to amino acid 501 of SEQ ID NO: 16 is I, the amino acid in the capsid protein corresponding to amino acid 505 of SEQ ID NO: 16 is R, and the amino acid in the capsid protein corresponding to amino acid 706 of SEQ ID NO: 16 is C. In certain embodiments, the capsid protein comprises the amino acid sequence of amino acids 1-736 of SEQ ID NO: 1, 2, 3, 4, 5, 6, 7, 8, 9, 10, 11, 12, 13, 15, 16, or 17.

In certain embodiments, the AAV capsid comprises two or more of: (a) a capsid protein comprising the amino acid sequence of amino acids 203-736 of SEQ ID NO: 1, 2, 3, 4, 6, 7, 10, 11, 12, 13, 15, 16, or 17; (b) a capsid protein comprising the amino acid sequence of amino acids 138-736 of SEQ ID NO: 1, 2, 3, 4, 5, 6, 7, 9, 10, 11, 12, 13, 15, 16, or 17; and (c) a capsid protein comprising the amino acid sequence of amino acids 1-736 of SEQ ID NO: 1, 2, 3, 4, 5, 6, 7, 8, 9, 10, 11, 12, 13, 15, 16, or 17. In certain embodiments, the AAV capsid comprises: (a) a capsid protein having an amino acid sequence consisting of amino acids 203-736 of SEQ ID NO: 1, 2, 3, 4, 6, 7, 10, 11, 12, 13, 15, 16, or 17; (b) a capsid protein having an amino acid sequence consisting of amino acids 138-736 of SEQ ID NO: 1, 2, 3, 4, 5, 6, 7, 9, 10, 11, 12, 13, 15, 16, or 17; and (c) a capsid protein having an amino acid sequence consisting of amino acids 1-736 of SEQ ID NO: 1, 2, 3, 4, 5, 6, 7, 8, 9, 10, 11, 12, 13, 15, 16, or 17.

In certain embodiments, the AAV capsid comprises one or more of: (a) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the sequence of amino acids 203-736 of SEQ ID NO: 8; (b) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the sequence of amino acids 138-736 of SEQ ID NO: 8; and (c) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the sequence of amino acids 1-736 of SEQ ID NO: 8. In certain embodiments, the AAV capsid comprises one or more of: (a) a capsid protein comprising the amino acid sequence of amino acids 203-736 of SEQ ID NO: 8; (b) a capsid protein comprising the amino acid sequence of amino acids 138-736 of SEQ ID NO: 8; and (c) a capsid protein comprising the amino acid sequence of amino acids 1-736 of SEQ ID NO: 8. In certain embodiments, the AAV capsid comprises two or more of: (a) a capsid protein comprising the amino acid sequence of amino acids 203-736 of SEQ ID NO: 8; (b) a capsid protein comprising the amino acid sequence of amino acids 138-736 of SEQ ID NO: 8; and (c) a capsid protein comprising the amino acid sequence of amino acids 1-736 of SEQ ID NO: 8. In certain embodiments, the AAV capsid comprises: (a) a capsid protein having an amino acid sequence consisting of amino acids 203-736 of SEQ ID NO: 8; (b) a capsid protein having an amino acid sequence consisting of amino acids 138-736 of SEQ ID NO: 8; and (c) a capsid protein having an amino acid sequence consisting of amino acids 1-736 of SEQ ID NO: 8.

In certain embodiments, the AAV capsid comprises one or more of: (a) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the sequence of amino acids 203-736 of SEQ ID NO: 11; (b) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the sequence of amino acids 138-736 of SEQ ID NO: 11; and (c) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the sequence of amino acids 1-736 of SEQ ID NO: 11. In certain embodiments, the AAV capsid comprises one or more of: (a) a capsid protein comprising the amino acid sequence of amino acids 203-736 of SEQ ID NO: 11; (b) a capsid protein comprising the amino acid sequence of amino acids 138-736 of SEQ ID NO: 11; and (c) a capsid protein comprising the amino acid sequence of amino acids 1-736 of SEQ ID NO: 11. In certain embodiments, the AAV capsid comprises two or more of: (a) a capsid protein comprising the amino acid sequence of amino acids 203-736 of SEQ ID NO: 11; (b) a capsid protein comprising the amino acid sequence of amino acids 138-736 of SEQ ID NO: 11; and (c) a capsid protein comprising the amino acid sequence of amino acids 1-736 of SEQ ID NO: 11. In certain embodiments, the AAV capsid comprises: (a) a capsid protein having an amino acid sequence consisting of amino acids 203-736 of SEQ ID NO: 11; (b) a capsid protein having an amino acid sequence consisting of amino acids 138-736 of SEQ ID NO: 11; and (c) a capsid protein having an amino acid sequence consisting of amino acids 1-736 of SEQ ID NO: 11.

In certain embodiments, the AAV capsid comprises one or more of: (a) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the sequence of amino acids 203-736 of SEQ ID NO: 13; (b) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the sequence of amino acids 138-736 of SEQ ID NO: 13; and (c) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the sequence of amino acids 1-736 of SEQ ID NO: 13. In certain embodiments, the AAV capsid comprises one or more of: (a) a capsid protein comprising the amino acid sequence of amino acids 203-736 of SEQ ID NO: 13; (b) a capsid protein comprising the amino acid sequence of amino acids 138-736 of SEQ ID NO: 13; and (c) a capsid protein comprising the amino acid sequence of amino acids 1-736 of SEQ ID NO: 13. In certain embodiments, the AAV capsid comprises two or more of: (a) a capsid protein comprising the amino acid sequence of amino acids 203-736 of SEQ ID NO: 13; (b) a capsid protein comprising the amino acid sequence of amino acids 138-736 of SEQ ID NO: 13; and (c) a capsid protein comprising the amino acid sequence of amino acids 1-736 of SEQ ID NO: 13. In certain embodiments, the AAV capsid comprises: (a) a capsid protein having an amino acid sequence consisting of amino acids 203-736 of SEQ ID NO: 13; (b) a capsid protein having an amino acid sequence consisting of amino acids 138-736 of SEQ ID NO: 13; and (c) a capsid protein having an amino acid sequence consisting of amino acids 1-736 of SEQ ID NO: 13.

In certain embodiments, the AAV capsid comprises one or more of: (a) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the sequence of amino acids 203-736 of SEQ ID NO: 16; (b) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the sequence of amino acids 138-736 of SEQ ID NO: 16; and (c) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the sequence of amino acids 1-736 of SEQ ID NO: 16. In certain embodiments, the AAV capsid comprises one or more of: (a) a capsid protein comprising the amino acid sequence of amino acids 203-736 of SEQ ID NO: 16; (b) a capsid protein comprising the amino acid sequence of amino acids 138-736 of SEQ ID NO: 16; and (c) a capsid protein comprising the amino acid sequence of amino acids 1-736 of SEQ ID NO: 16. In certain embodiments, the AAV capsid comprises two or more of: (a) a capsid protein comprising the amino acid sequence of amino acids 203-736 of SEQ ID NO: 16; (b) a capsid protein comprising the amino acid sequence of amino acids 138-736 of SEQ ID NO: 16; and (c) a capsid protein comprising the amino acid sequence of amino acids 1-736 of SEQ ID NO: 16. In certain embodiments, the AAV capsid comprises: (a) a capsid protein having an amino acid sequence consisting of amino acids 203-736 of SEQ ID NO: 16; (b) a capsid protein having an amino acid sequence consisting of amino acids 138-736 of SEQ ID NO: 16; and (c) a capsid protein having an amino acid sequence consisting of amino acids 1-736 of SEQ ID NO: 16.

The rAAV genomes of the disclosure can be used to express any antibody heavy chain and antibody light chain known in the art. In one aspect, provided herein are rAAV genomes comprising a TRE operably linked to at least a portion of an antibody coding sequence (e.g., an antibody heavy chain coding sequence and/or an antibody light chain coding sequence). Non-limiting examples of antibodies include, anti-C5 antibodies (e.g., eculizumab, ravulizumab, and pozelimab), anti-Factor D antibodies (e.g., lampalizumab), anti-mannose-binding protein-associated serine protease 2 (MASP-2) antibodies (e.g., narsoplimab), anti-kallikrein antibodies (e.g., lanadelumab), anti-interleukin 1 beta antibodies (e.g., canakinumab), anti-interferon gamma antibodies (e.g., emapalumab), anti-PC SK9 antibodies (e.g., evolocumab and alirocumab), anti-coagulation factor IX and factor X antibodies (e.g., bispecific antibody emicizumab), and anti-VEGF antibodies (e.g., ranibizumab).

In another aspect, the instant disclosure provides pharmaceutical compositions comprising an AAV as disclosed herein together with a pharmaceutically acceptable excipient, adjuvant, diluent, vehicle or carrier, or a combination thereof. A “pharmaceutically acceptable carrier” includes any material which, when combined with an active ingredient of a composition, allows the ingredient to retain biological activity and without causing disruptive physiological reactions, such as an unintended immune reaction. Pharmaceutically acceptable carriers include water, phosphate buffered saline, emulsions such as oil/water emulsion, and wetting agents. Compositions comprising such carriers are formulated by well-known conventional methods such as those set forth in Remington's Pharmaceutical Sciences, current Ed., Mack Publishing Co., Easton Pa. 18042, USA; A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al, 7th ed., Lippincott, Williams, & Wilkins; and Handbook of Pharmaceutical Excipients (2000) A. H. Kibbe et al, 3rd ed. Amer. Pharmaceutical Assoc.

In another aspect, the instant disclosure provides pharmaceutical compositions comprising an AAV as disclosed herein together with a pharmaceutically acceptable excipient, adjuvant, diluent, vehicle or carrier, or a combination thereof. A “pharmaceutically acceptable carrier” includes any material which, when combined with an active ingredient of a composition, allows the ingredient to retain biological activity and without causing disruptive physiological reactions, such as an unintended immune reaction. Pharmaceutically acceptable carriers include water, phosphate buffered saline, emulsions such as oil/water emulsion, and wetting agents. Compositions comprising such carriers are formulated by well-known conventional methods such as those set forth in Remington's Pharmaceutical Sciences, current Ed., Mack Publishing Co., Easton Pa. 18042, USA; A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al, 7th ed., Lippincott, Williams, & Wilkins; and Handbook of Pharmaceutical Excipients (2000) A. H. Kibbe et al, 3rd ed. Amer. Pharmaceutical Assoc.

### III. METHOD OF USE

In another aspect, the instant disclosure provides methods for expressing an antibody in a cell (e.g., an antibody heavy chain and light chain). The methods generally comprise transducing the cell with an rAAV as disclosed herein. Such methods lead to high-level expression and secretion of antibodies. Accordingly, in certain embodiments, the methods disclosed herein involve transducing the cell with an rAAV as disclosed herein.

The methods disclosed herein can be applied to any cell (e.g., liver cells) in which expression of an antibody is desired. Accordingly, in certain embodiments, the method is applied to cells in the liver. In certain embodiments, the method is applied to hepatocytes.

The methods disclosed herein can be performed in vitro for research purposes or can be performed ex vivo or in vivo for therapeutic purposes.

In certain embodiments, the cell to be transduced is in a mammalian subject and the AAV is administered to the subject in an amount effective to transduce the cell in the subject. Accordingly, in certain embodiments, the instant disclosure provides a method for treating a subject having a disease or disorder that would benefit from the expression and secretion of an antibody that specifically binds a therapeutic target, the method generally comprising administering to the subject an effective amount of an rAAV as disclosed herein. In certain embodiments, the antibody specifically binds complement C5 and the disease or disorder is associated with complement C5 activity. The subject can be a human subject or a rodent subject (e.g., a mouse) containing human liver cells. Suitable mouse subjects include without limitation, mice into which human liver cells (e.g., human hepatocytes) have been engrafted. Any disease or disorder associated with complement C5 activity can be treated using the methods disclosed herein. Suitable diseases or disorders include, without limitation, paroxysmal nocturnal hemoglobinuria (PNH), neuromyelitis optica spectrum disorder (NMOSD), atypical hemolytic uremic syndrome (aHUS), myasthenia gravis, hematopoietic stem cell transplantation-transplant-associated thrombotic microangiopathy (HSCT-TMA), complement-mediated thrombotic microangiopathy (CM-TMA), Guillain-Barré syndrome, amyotrophic lateral sclerosis (ALS), primary progressive multiple sclerosis (PPMS), multifocal motor neuropathy, antibody-mediated kidney rejection, C3 glomerulopathy, age-related macular degeneration (AMD), AQP4 IgG-positive neuromyelitis optica, systemic lupus erythematosus, psoriasis, rheumatoid arthritis (RA), dermatomyositis, idiopathic membranous glomerulopathy, demyelinating neuropathy, complement hyperactivation, angiopathic thrombosis, protein losing enteropathy (CHAPLE) syndrome, geographic atrophy (GA), asthma, proliferative nephritis, and sepsis.

In certain embodiments, the instant disclosure provides a method for treating a subject having a disease or disorder associated with complement C5 activity, the method generally comprising administering to the subject an effective amount of an rAAV as disclosed herein. The subject can be a human subject, a non-human primate subject (e.g., a cynomolgus), or a rodent subject (e.g., a mouse) with aberrant complement C5 activity. Any disease or disorder associated with complement C5 activity can be treated using the methods disclosed herein. Suitable diseases or disorders include, without limitation, PNH, NMOSD, aHUS, myasthenia gravis, HSCT-TMA, CM-TMA, Guillain-Barré syndrome, ALS, PPMS, multifocal motor neuropathy, antibody-mediated kidney rejection, C3 glomerulopathy, AMD, AQP4 IgG-positive neuromyelitis optica, systemic lupus erythematosus, psoriasis, RA, dermatomyositis, idiopathic membranous glomerulopathy, demyelinating neuropathy, CHAPLE syndrome, geographic atrophy (GA), asthma, proliferative nephritis, and sepsis.

In certain embodiments, the foregoing methods employ an rAAV comprising an AAV capsid protein comprising the amino acid sequence of amino acids 203-736 of SEQ ID NO: 16, and an rAAV genome comprising 5′ to 3′ following genetic elements: a 5′ ITR (e.g., the 5′ ITR comprising the nucleotide sequence set forth in SEQ ID NO: 14), a transcriptional regulatory element (e.g., a TRE comprising the nucleotide sequence set forth in SEQ ID NO: 60), at least a portion of an antibody heavy chain coding sequence (e.g., the antibody heavy chain coding sequence comprising the nucleotide sequence set forth in SEQ ID NO: 52, 62, or 83), a T2A peptide cleavage sequence (e.g., the T2A peptide cleavage sequence of SEQ ID NO: 28), at least a portion of an antibody light chain coding sequence (e.g., the antibody light chain coding sequence comprising the nucleotide sequence set forth in SEQ ID NO: 53 or 63), a polyadenylation sequence (e.g., the SV40 polyadenylation sequence of SEQ ID NO: 31), and a 3′ ITR (e.g., the 3′ ITR comprising the nucleotide sequence set forth in SEQ ID NO: 18).

In certain embodiments, the foregoing methods employ an rAAV comprising an AAV capsid protein comprising the amino acid sequence of amino acids 138-736 of SEQ ID NO: 16, and an rAAV genome comprising 5′ to 3′ following genetic elements: a 5′ ITR (e.g., the 5′ ITR comprising the nucleotide sequence set forth in SEQ ID NO: 14), a transcriptional regulatory element (e.g., a TRE comprising the nucleotide sequence set forth in SEQ ID NO: 27), an intron element (e.g., the intron element comprising the nucleotide sequence set forth in SEQ ID NO: 29), at least a portion of an antibody heavy chain coding sequence (e.g., the antibody heavy chain coding sequence comprising the nucleotide sequence set forth in SEQ ID NO: 52, 62, or 83), a T2A peptide cleavage sequence (e.g., the T2A peptide cleavage sequence of SEQ ID NO: 28), at least a portion of an antibody light chain coding sequence (e.g., the antibody light chain coding sequence comprising the nucleotide sequence set forth in SEQ ID NO: 53 or 63), a polyadenylation sequence (e.g., the SV40 polyadenylation sequence of SEQ ID NO: 31), and a 3′ ITR (e.g., the 3′ ITR comprising the nucleotide sequence set forth in SEQ ID NO: 18).

In certain embodiments, the foregoing methods employ an rAAV comprising an AAV capsid protein comprising the amino acid sequence of SEQ ID NO: 16, and an rAAV genome comprising 5′ to 3′ following genetic elements: a 5′ ITR (e.g., the 5′ ITR comprising the nucleotide sequence set forth in SEQ ID NO: 14), a transcriptional regulatory element (e.g., a TRE comprising the nucleotide sequence set forth in SEQ ID NO: 27), an intron element (e.g., the intron element comprising the nucleotide sequence set forth in SEQ ID NO: 29), at least a portion of an antibody heavy chain coding sequence (e.g., the antibody heavy chain coding sequence comprising the nucleotide sequence set forth in SEQ ID NO: 52, 62, or 83), a polyadenylation sequence (e.g., the BGH polyadenylation sequence of SEQ ID NO: 33), a transcriptional regulatory element (e.g., a TRE comprising the nucleotide sequence set forth in SEQ ID NO: 67), an intron element (e.g., the intron element comprising the nucleotide sequence set forth in SEQ ID NO: 30 or 61), at least a portion of an antibody light chain coding sequence (e.g., the antibody light chain coding sequence comprising the nucleotide sequence set forth in SEQ ID NO: 53 or 63), a polyadenylation sequence (e.g., the SV40 polyadenylation sequence of SEQ ID NO: 31), and a 3′ ITR (e.g., the 3′ ITR comprising the nucleotide sequence set forth in SEQ ID NO: 18).

In certain embodiments, the foregoing methods employ an rAAV comprising an AAV capsid protein comprising the amino acid sequence of SEQ ID NO: 16, and an rAAV genome comprising 5′ to 3′ following genetic elements: a 5′ ITR (e.g., the 5′ ITR comprising the nucleotide sequence set forth in SEQ ID NO: 14), a polyadenylation sequence (e.g., the BGH polyadenylation sequence of SEQ ID NO: 33), at least a portion of an antibody heavy chain coding sequence (e.g., the antibody heavy chain coding sequence comprising the nucleotide sequence set forth in SEQ ID NO: 52, 62, or 83), an intron element (e.g., the intron element comprising the nucleotide sequence set forth in SEQ ID NO: 29), a transcriptional regulatory element (e.g., a TRE comprising the nucleotide sequence set forth in SEQ ID NO: 27), a stuffer sequence (e.g., a stuffer comprising the nucleotide sequence set forth in SEQ ID NO: 51), a transcriptional regulatory element (e.g., a TRE comprising the nucleotide sequence set forth in SEQ ID NO: 67), an intron element (e.g., the intron element comprising the nucleotide sequence set forth in SEQ ID NO: 30 or 61), at least a portion of an antibody light chain coding sequence (e.g., the antibody light chain coding sequence comprising the nucleotide sequence set forth in SEQ ID NO: 53 or 63), a polyadenylation sequence (e.g., the SV40 polyadenylation sequence of SEQ ID NO: 31), and a 3′ ITR (e.g., the 3′ ITR comprising the nucleotide sequence set forth in SEQ ID NO: 18).

In certain embodiments, the foregoing methods employ an rAAV comprising an AAV capsid protein comprising the amino acid sequence of amino acids 203-736 of SEQ ID NO: 13, and an rAAV genome comprising 5′ to 3′ following genetic elements: a 5′ ITR (e.g., the 5′ ITR comprising the nucleotide sequence set forth in SEQ ID NO: 14), a transcriptional regulatory element (e.g., a TRE comprising the nucleotide sequence set forth in SEQ ID NO: 60), at least a portion of an antibody heavy chain coding sequence (e.g., the antibody heavy chain coding sequence comprising the nucleotide sequence set forth in SEQ ID NO: 52, 62, or 83), a T2A peptide cleavage sequence (e.g., the T2A peptide cleavage sequence of SEQ ID NO: 28), at least a portion of an antibody light chain coding sequence (e.g., the antibody light chain coding sequence comprising the nucleotide sequence set forth in SEQ ID NO: 53 or 63), a polyadenylation sequence (e.g., the SV40 polyadenylation sequence of SEQ ID NO: 31), and a 3′ ITR (e.g., the 3′ ITR comprising the nucleotide sequence set forth in SEQ ID NO: 18).

In certain embodiments, the foregoing methods employ an rAAV comprising an AAV capsid protein comprising the amino acid sequence of amino acids 138-736 of SEQ ID NO: 13, and an rAAV genome comprising 5′ to 3′ following genetic elements: a 5′ ITR (e.g., the 5′ ITR comprising the nucleotide sequence set forth in SEQ ID NO: 14), a transcriptional regulatory element (e.g., a TRE comprising the nucleotide sequence set forth in SEQ ID NO: 27), an intron element (e.g., the intron element comprising the nucleotide sequence set forth in SEQ ID NO: 29), at least a portion of an antibody heavy chain coding sequence (e.g., the antibody heavy chain coding sequence comprising the nucleotide sequence set forth in SEQ ID NO: 52, 62, or 83), a T2A peptide cleavage sequence (e.g., the T2A peptide cleavage sequence of SEQ ID NO: 28), at least a portion of an antibody light chain coding sequence (e.g., the antibody light chain coding sequence comprising the nucleotide sequence set forth in SEQ ID NO: 53 or 63), a polyadenylation sequence (e.g., the SV40 polyadenylation sequence of SEQ ID NO: 31), and a 3′ ITR (e.g., the 3′ ITR comprising the nucleotide sequence set forth in SEQ ID NO: 18).

In certain embodiments, the foregoing methods employ an rAAV comprising an AAV capsid protein comprising the amino acid sequence of SEQ ID NO: 13, and an rAAV genome comprising 5′ to 3′ following genetic elements: a 5′ ITR (e.g., the 5′ ITR comprising the nucleotide sequence set forth in SEQ ID NO: 14), a transcriptional regulatory element (e.g., a TRE comprising the nucleotide sequence set forth in SEQ ID NO: 27), an intron element (e.g., the intron element comprising the nucleotide sequence set forth in SEQ ID NO: 29), at least a portion of an antibody heavy chain coding sequence (e.g., the antibody heavy chain coding sequence comprising the nucleotide sequence set forth in SEQ ID NO: 52, 62, or 83), a polyadenylation sequence (e.g., the BGH polyadenylation sequence of SEQ ID NO: 33), a transcriptional regulatory element (e.g., a TRE comprising the nucleotide sequence set forth in SEQ ID NO: 67), an intron element (e.g., the intron element comprising the nucleotide sequence set forth in SEQ ID NO: 30 or 61), at least a portion of an antibody light chain coding sequence (e.g., the antibody light chain coding sequence comprising the nucleotide sequence set forth in SEQ ID NO: 53 or 63), a polyadenylation sequence (e.g., the SV40 polyadenylation sequence of SEQ ID NO: 31), and a 3′ ITR (e.g., the 3′ ITR comprising the nucleotide sequence set forth in SEQ ID NO: 18).

In certain embodiments, the foregoing methods employ an rAAV comprising an AAV capsid protein comprising the amino acid sequence of SEQ ID NO: 13, and an rAAV genome comprising 5′ to 3′ following genetic elements: a 5′ ITR (e.g., the 5′ ITR comprising the nucleotide sequence set forth in SEQ ID NO: 14), a polyadenylation sequence (e.g., the BGH polyadenylation sequence of SEQ ID NO: 33), at least a portion of an antibody heavy chain coding sequence (e.g., the antibody heavy chain coding sequence comprising the nucleotide sequence set forth in SEQ ID NO: 52, 62, or 83), an intron element (e.g., the intron element comprising the nucleotide sequence set forth in SEQ ID NO: 29), a transcriptional regulatory element (e.g., a TRE comprising the nucleotide sequence set forth in SEQ ID NO: 27), a stuffer sequence (e.g., a stuffer comprising the nucleotide sequence set forth in SEQ ID NO: 51), a transcriptional regulatory element (e.g., a TRE comprising the nucleotide sequence set forth in SEQ ID NO: 67), an intron element (e.g., the intron element comprising the nucleotide sequence set forth in SEQ ID NO: 30 or 61), at least a portion of an antibody light chain coding sequence (e.g., the antibody light chain coding sequence comprising the nucleotide sequence set forth in SEQ ID NO: 53 or 63), a polyadenylation sequence (e.g., the SV40 polyadenylation sequence of SEQ ID NO: 31), and a 3′ ITR (e.g., the 3′ ITR comprising the nucleotide sequence set forth in SEQ ID NO: 18).

The methods disclosed herein are particularly advantageous in that they are capable of expressing and secreting an antibody into the serum of a subject with high efficiency in vivo. In certain embodiments, the serum concentrations of the antibody is at least about 100 μg/mL, at least about 500 μg/mL, at least about 1000 μg/mL, at least about 1500 μg/mL, at least about 2000 μg/mL, at least about 2500 μg/mL, at least about 3000 μg/mL, at least about 3500 μg/mL, at least about 4000 μg/mL, at least about 4500 μg/mL, at least about 5000 μg/mL, at least about 7500 μg/mL, at least about 10000 μg/mL, at least about 15000 μg/mL, at least about 20000 μg/mL, at least about 25000 μg/mL, at least about 30000 μg/mL, at least about 35000 μg/mL, at least about 40000 μg/mL, at least about 45000 μg/mL, at least about 50000 μg/mL, at least about 60000 μg/mL, at least about 70000 μg/mL, at least about 80000 μg/mL, at least about 90000 μg/mL, or at least about 100000 μg/mL. Any methods of determining the expression level or serum concentration of the antibody can be employed including, without limitation, ELISA, Western blotting, immunostaining, and mass spectrometry. In certain embodiments, the serum concentration of the antibody is determined with an anti-human IgG ELISA.

In certain embodiments, transduction of a cell with an AAV composition disclosed herein can be performed as provided herein or by any method of transduction known to one of ordinary skill in the art. In certain embodiments, the cell may be contacted with the AAV at a multiplicity of infection (MOI) of 50,000; 100,000; 150,000; 200,000; 250,000; 300,000; 350,000; 400,000; 450,000; or 500,000, or at any MOI that provides for optimal transduction of the cell.

An AAV composition disclosed herein can be administered to a subject by any appropriate route including, without limitation, intravenous, intraperitoneal, subcutaneous, intramuscular, intranasal, topical or intradermal routes. In certain embodiments, the composition is formulated for administration via intravenous injection or subcutaneous injection.

### IV. AAV PACKAGING SYSTEMS

In another aspect, the instant disclosure provides packaging systems for recombinant preparation of a recombinant adeno-associated virus (rAAV) disclosed herein. Such packaging systems generally comprise: first nucleotide encoding one or more AAV Rep proteins; a second nucleotide encoding a capsid protein of any of the AAVs as disclosed herein; and a third nucleotide sequence comprising any of the rAAV genomes as disclosed herein, wherein the packaging system is operative in a cell for enclosing the rAAV genome in the capsid to form the AAV.

In certain embodiments, the packaging system comprises a first vector comprising the first nucleotide sequence encoding the one or more AAV Rep proteins and the second nucleotide sequence encoding the AAV capsid protein, and a second vector comprising the third nucleotide sequence comprising the rAAV genome. As used in the context of a packaging system as described herein, a “vector” refers to a nucleic acid molecule that is a vehicle for introducing nucleic acids into a cell (e.g., a plasmid, a virus, a cosmid, an artificial chromosome, etc.).

Any AAV Rep protein can be employed in the packaging systems disclosed herein. In certain embodiments of the packaging system, the Rep nucleotide sequence encodes an AAV2 Rep protein. Suitable AAV2 Rep proteins include, without limitation, Rep 78/68 or Rep 68/52. In certain embodiments of the packaging system, the nucleotide sequence encoding the AAV2 Rep protein comprises a nucleotide sequence that encodes a protein having a minimum percent sequence identity to the AAV2 Rep amino acid sequence of SEQ ID NO: 22, wherein the minimum percent sequence identity is at least 70% (e.g., at least 75%, at least 80%, at least 85%, at least 90%, at least 95%, at least 98%, at least 99%, or 100%) across the length of the amino acid sequence of the AAV2 Rep protein. In certain embodiments of the packaging system, the AAV2 Rep protein has the amino acid sequence set forth in SEQ ID NO: 22.

In certain embodiments of the packaging system, the packaging system further comprises a fourth nucleotide sequence comprising one or more helper virus genes. In certain embodiments of the packaging system, the packaging system further comprises a third vector, e.g., a helper virus vector, comprising the fourth nucleotide sequence comprising the one or more helper virus genes. The third vector may be an independent third vector, integral with the first vector, or integral with the second vector.

In certain embodiments of the packaging system, the helper virus is selected from the group consisting of adenovirus, herpes virus (including herpes simplex virus (HSV)), poxvirus (such as vaccinia virus), cytomegalovirus (CMV), and baculovirus. In certain embodiments of the packaging system, where the helper virus is adenovirus, the adenovirus genome comprises one or more adenovirus RNA genes selected from the group consisting of E1, E2, E4 and VA. In certain embodiments of the packaging system, where the helper virus is HSV, the HSV genome comprises one or more of HSV genes selected from the group consisting of UL5/8/52, ICPO, ICP4, ICP22 and UL30/UL42.

In certain embodiments of the packaging system, the first, second, and/or third vector are contained within one or more plasmids. In certain embodiments, the first vector and the third vector are contained within a first plasmid. In certain embodiments the second vector and the third vector are contained within a second plasmid.

In certain embodiments of the packaging system, the first, second, and/or third vector are contained within one or more recombinant helper viruses. In certain embodiments, the first vector and the third vector are contained within a recombinant helper virus. In certain embodiments, the second vector and the third vector are contained within a recombinant helper virus.

In a further aspect, the disclosure provides a method for recombinant preparation of an AAV as described herein, wherein the method comprises transfecting or transducing a cell with a packaging system as described herein under conditions operative for enclosing the rAAV genome in the capsid to form the rAAV as described herein. Exemplary methods for recombinant preparation of an rAAV include transient transfection (e.g., with one or more transfection plasmids containing a first, and a second, and optionally a third vector as described herein), viral infection (e.g. with one or more recombinant helper viruses, such as a adenovirus, poxvirus (such as vaccinia virus), herpes virus (including HSV, cytomegalovirus, or baculovirus, containing a first, and a second, and optionally a third vector as described herein), and stable producer cell line transfection or infection (e.g., with a stable producer cell, such as a mammalian or insect cell, containing a Rep nucleotide sequence encoding one or more AAV Rep proteins and/or a Cap nucleotide sequence encoding one or more AAV capsid proteins as described herein, and with an rAAV genome as described herein being delivered in the form of a plasmid or a recombinant helper virus).

Accordingly, the instant disclosure provides a packaging system for preparation of a recombinant AAV (rAAV), wherein the packaging system comprises a first nucleotide sequence encoding one or more AAV Rep proteins; a second nucleotide sequence encoding a capsid protein of any one of the AAVs described herein; a third nucleotide sequence comprising an rAAV genome sequence of any one of the AAVs described herein; and optionally a fourth nucleotide sequence comprising one or more helper virus genes.

### V. EXAMPLES

The following examples demonstrate the efficient expression of antibodies (e.g., anti-C5 antibodies) in a subject using an rAAV vector as disclosed herein. These examples are offered by way of illustration, and not by way of limitation.

### Example 1: Anti-Complement C5 Antibody rAAV Vectors

This example provides anti-C5 antibody expressing vectors C5Ab01, C5Ab02, C5Ab03, and C5Ab04 for expression of anti-C5 antibodies in a cell (e.g., a human cell or a mouse cell) into which the vector is transduced.

### a) C5Ab01

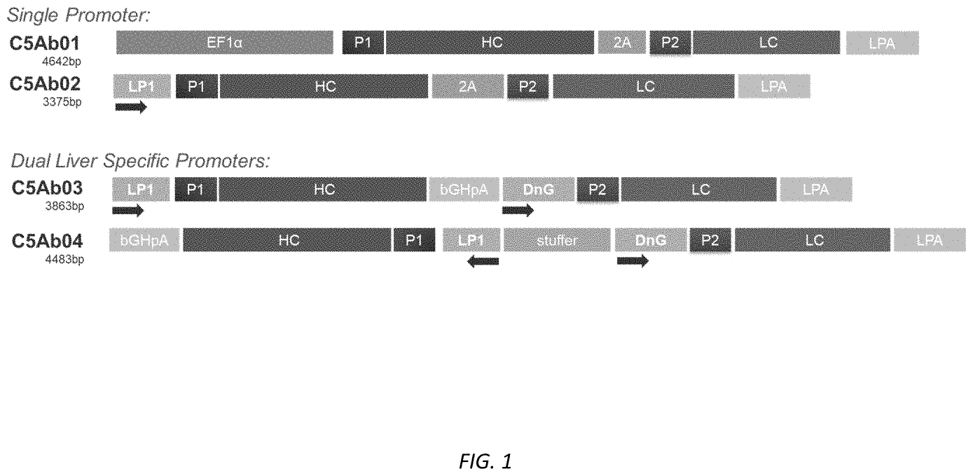

Anti-C5 antibody vector C5Ab01, as shown in FIG. 1, comprises, from 5′ to 3′, the following genetic elements: a transcriptional regulatory element comprising an EF1α promoter; a coding sequence encoding a human IgG2 (P1) signal sequence linked to an anti-C5 antibody heavy chain (HC); a nucleic acid sequence encoding a 2A ribosomal skipping peptide; a coding sequence encoding an Igκ (P2) signal sequence linked to an anti-C5 antibody light chain (LC); and an SV40 late polyadenylation sequence (LPA). The nucleic sequences of these elements are set forth in Table 1. This vector is capable of expressing an anti-C5 antibody in a cell (e.g., a human cell or a mouse cell) into which the vector is transduced.

### b) C5Ab02

Anti-C5 antibody vector C5Ab02, as shown in FIG. 1, comprises, from 5′ to 3′, the following genetic elements: a transcriptional regulatory element comprising the liver-specific LP1 promoter; a coding sequence encoding a human IgG2 (P1) signal sequence linked to an anti-C5 antibody heavy chain (HC); a nucleic acid sequence encoding 2A ribosomal skipping peptide; a coding sequence encoding an Igκ (P2) signal sequence linked to an anti-C5 antibody light chain (LC); and an SV40 late polyadenylation sequence (LPA). The sequences of these elements are set forth in Table 1. This vector is capable of expressing an anti-C5 antibody in a cell (e.g., a human cell or a mouse cell) into which the vector is transduced.

### c) C5Ab03

Anti-C5 antibody vector C5Ab03, as shown in FIG. 1, comprises, from 5′ to 3′, the following genetic elements: a transcriptional regulatory element comprising the liver-specific LP1 promoter; a coding sequence encoding a human IgG2 (P1) signal sequence linked to an anti-C5 antibody heavy chain (HC); a bovine growth hormone polyadenylation signal (bGHpA); a transcriptional regulatory element comprising the liver-specific DnG promoter; a coding sequence encoding an Igκ (P2) signal sequence linked to an anti-C5 antibody light chain (LC); and an SV40 late polyadenylation sequence (LPA). The sequences of these elements are set forth in Table 1. This vector is capable of expressing an anti-C5 antibody in a cell (e.g., a human cell or a mouse cell) into which the vector is transduced.

### d) C5Ab04

Anti-C5 antibody vector C5Ab04, as shown in FIG. 1, comprises from 5′ to 3′, the following genetic elements: a bovine growth hormone polyadenylation signal (bGHpA); an anti-C5 antibody heavy chain coding sequence; a coding sequence encoding an anti-C5 antibody heavy chain (HC) linked to a human IgG2 (P1) signal sequence; a transcriptional regulatory element comprising the liver-specific LP1 promoter; a stuffer sequence; a transcriptional regulatory element comprising the liver-specific DnG promoter; a coding sequence encoding an Igκ (P2) signal sequence linked to an anti-C5 antibody light chain (LC); and an SV40 late polyadenylation sequence (LPA). The sequences of these elements are set forth in Table 1. This vector is capable of expressing an anti-C5 antibody in a cell (e.g., a human cell or a mouse cell) into which the vector is transduced.

TABLE 1
Genetic elements in anti-C5 antibody expressing vectors
C5Ab01, C5Ab02, C5Ab02, C5Ab03, and C5Ab04

| Genetic element | C5Ab01 | C5Ab02 | C5Ab03 | C5Ab04 |
|---|---|---|---|---|
| (from 5′ to 3′) | SEQ ID NO | | | |
| 5′ ITR element | 14 | 14 | 14 | 14 |
| First Transcriptional | 60 | 50 | 50 | 50 |
| regulatory element | | | | |
| Antibody heavy chain | 23 | 23 | 23 | 23 |
| signal sequence | | | | |
| Anti-C5 antibody | 52 | 52 | 52 | 52 |
| heavy chain coding | | | | |
| sequence | | | | |
| First Polyadenylation | N/A | N/A | 33 | 33 |
| Sequence | | | | |
| Second Transcriptional | N/A | N/A | 43 | 43 |
| regulatory element | | | | |
| Antibody light chain | 24 | 24 | 24 | 24 |
| signal sequence | | | | |
| Anti-C5 antibody | 53 | 53 | 53 | 53 |
| light chain coding | | | | |
| sequence | | | | |
| Second Polyadenylation | 31 | 31 | 31 | 31 |
| Sequence | | | | |
| 3′ ITR element | 18 | 18 | 18 | 18 |
| rAAV genome | 84 | 85 | 86 | 87 |
| (from promoter to | | | | |
| polyA sequence) | | | | |
| rAAV genome | 88 | 89 | 90 | 91 |
| (from 5′ ITR to | | | | |
| 3′ ITR) | | | | |

The vectors disclosed herein can be packaged in an AAV capsid, including, without limitation, an AAVHSC5, AAVHSC7, AAVHSC8, AAVHSC13, AAVHSC15, or AAVHSC17 capsid.

### Example 2: Expression of Anti-C5 Antibodies in a Mouse Model

Aberrant or excessive activity of the complement component C5 is associated with several diseases, including paroxysmal nocturnal hemoglobinuria (PNH), neuromyelitis optica spectrum disorder, (NMOSD), and atypical hemolytic uremic syndrome (aHUS). Anti-C5 monoclonal antibodies have been shown to be effective in treating these diseases, but patients often require multiple large doses of the antibody to enjoy the therapeutic benefits. This is, in part, due to the high concentration of C5 protein in the patient's serum. It, therefore, requires high levels anti-C5 antibodies to bind and eliminate enough C5 to produce the required therapeutic effect. These issues may be overcome if a patient is capable of expressing their own anti-C5 antibodies.

To study if sufficiently high levels of anti-C5 antibodies can be expressed in an organism, NOD SCID mice were administered AAV vectors for the expression of said anti-C5 antibodies from the mouse liver. Four separate experiments were performed, testing vectors C5Ab02, C5Ab03, and C5Ab04 (described above), packaged in each of AAVHSC13, AAVHSC15, and AAVHSC17.

### Human IgG ELISA Protocol

To evaluate serum human IgG concentration (μg/mL) in the following experiments, the SimpleStep ELISA® kit from Abcam was employed. Briefly, an antibody cocktail was prepared by diluting the capture and detector antibodies in Antibody Diluent CP. To make 3 mL of the antibody cocktail, 300 μL of 10× Capture Antibody and 300 μL 10× Detector Antibody were combined with 2.4 mL Antibody Diluent CP. Standards were subsequently prepared by serial dilution immediately prior to use. Human IgG protein provided in the kit was used for the positive control serial dilution.

To conduct the assay, all reagents were brought to room temperature prior to use. 50 μL of all samples or standards were added to appropriate wells of a microplate. 50 μL of the antibody cocktail was then added to each well. The plate was then sealed and incubated for 40 minutes at room temperature on a plate shaker set to 400 rpm. Each well was then washed with 3×350 μL 1× Wash Buffer PT. After the last wash, the plate was inverted and blotted against clean paper towels to remove excess liquid. 100 μL of TMB Development Solution was then added to each well and incubated for about 5 minutes in the dark on a plate shaker set to 400 rpm. Optimal incubation time may vary between 5 and 20 minutes. 100 μL of Stop Solution was added to each well. The plate was shaken on a plate shaker for 1 minute to mix. The optical density (OD) was then read at 450 nm. This was the endpoint reading.

Results

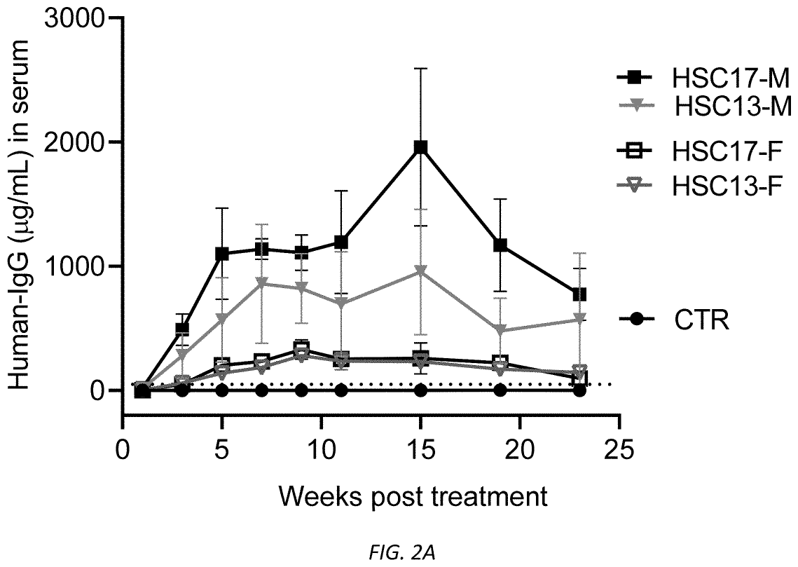

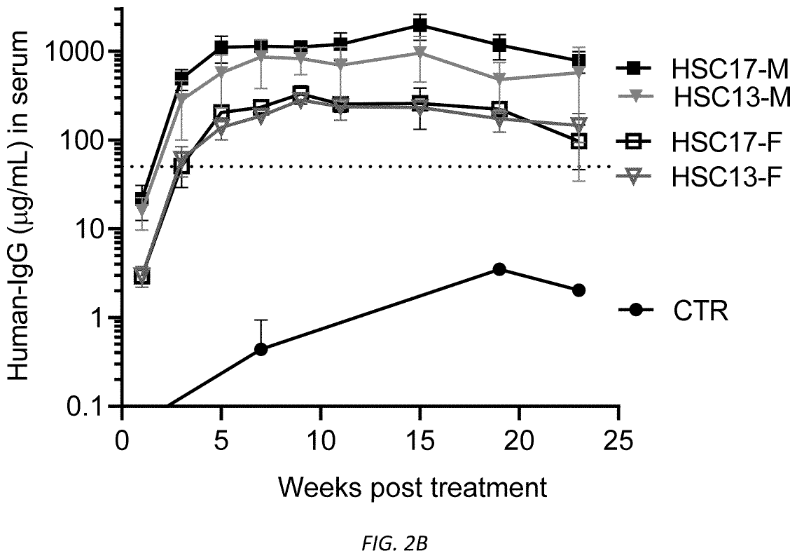

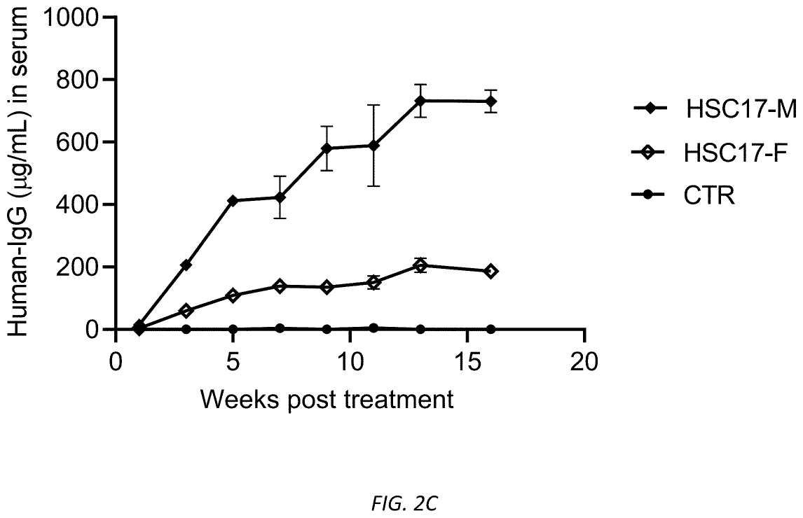

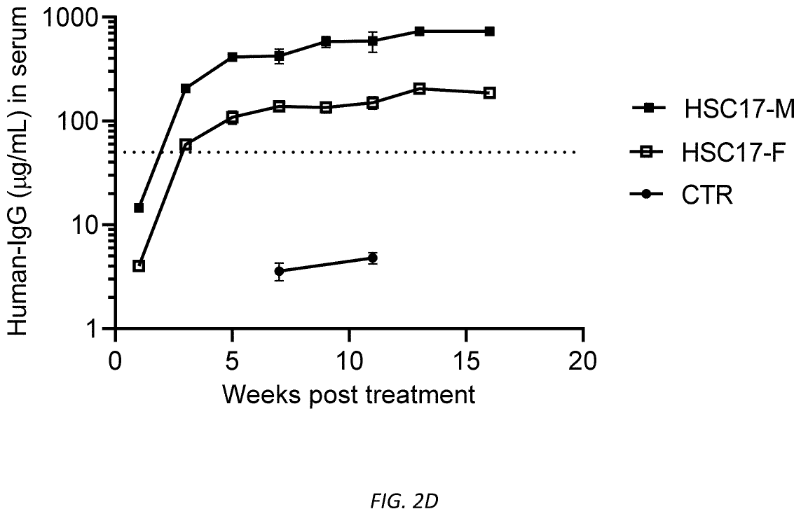

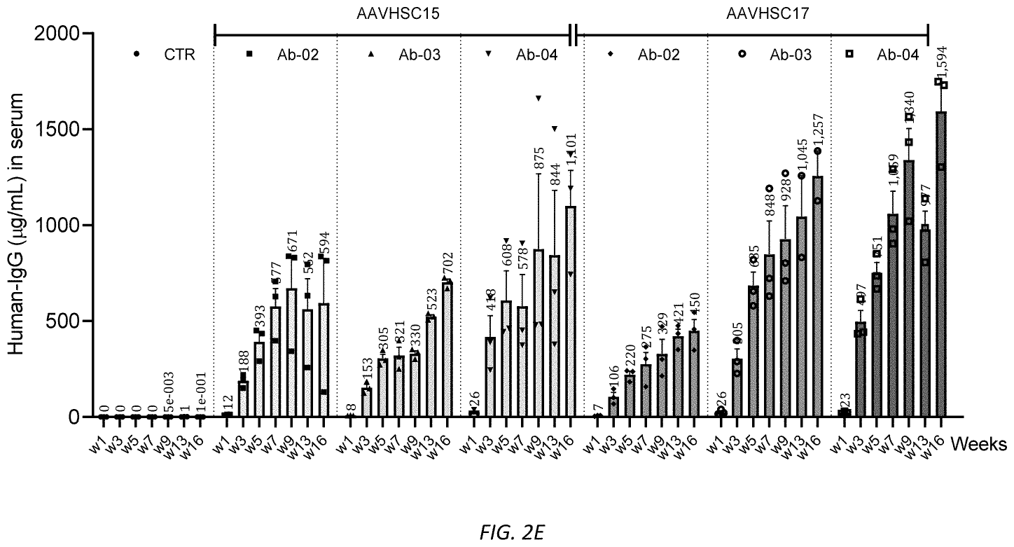

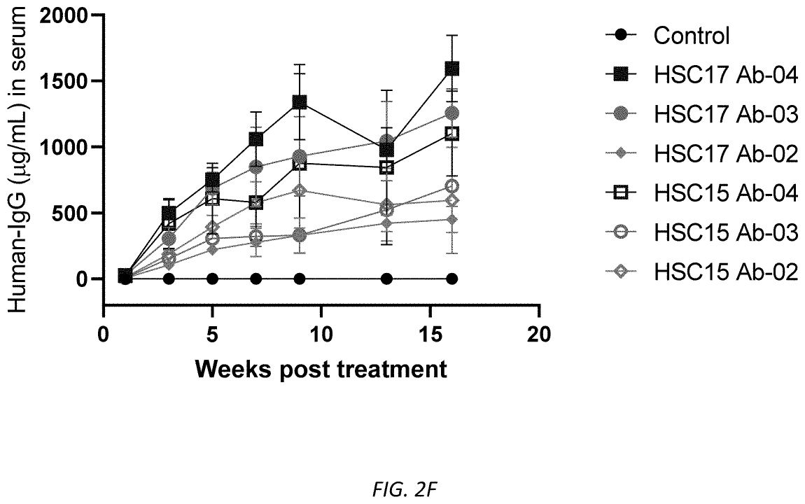

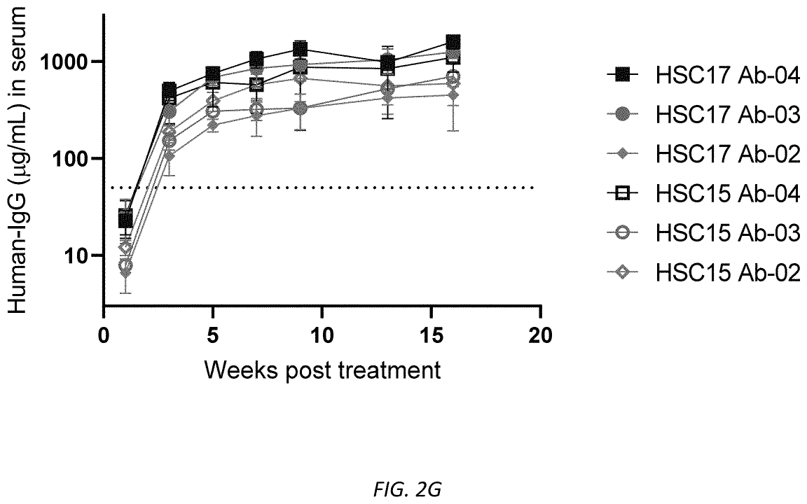

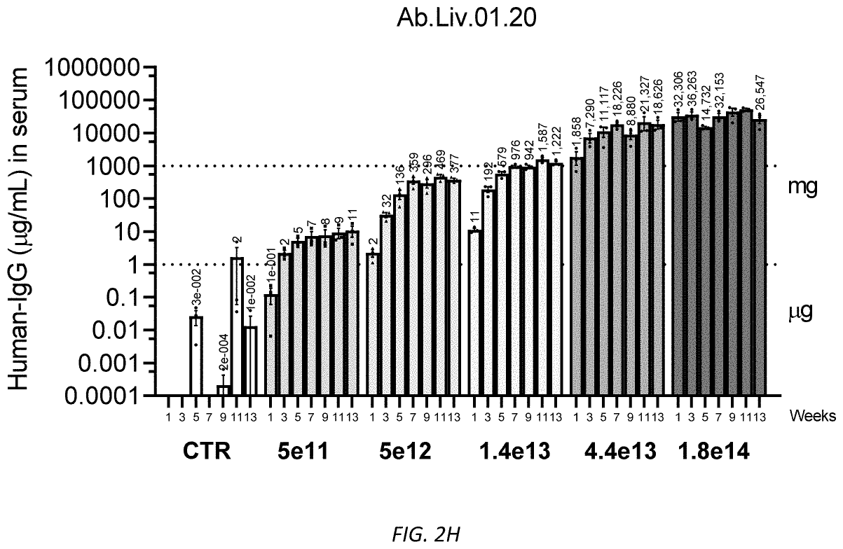

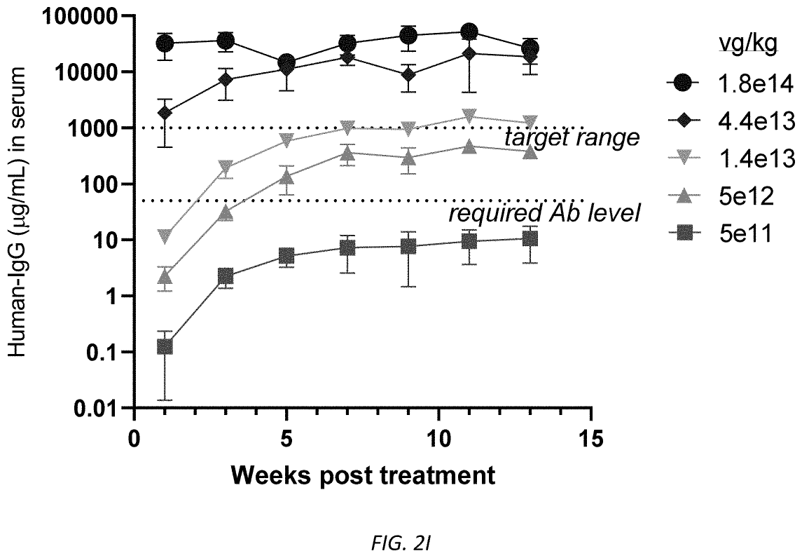

In a first experiment, mice received vector C5Ab04 packaged in the AAVHSC13 or the AAVHSC17 capsid at a dose of 1e13 vgs/kg. Serum samples were taken after 1 week, 3 weeks, 5 weeks, 7 weeks, 9 weeks, 11 weeks, 15 weeks, 19 weeks, and 23 weeks. The serum samples were tested for human IgG concentration (μg/mL) as a readout of anti-C5 antibody levels. Female mice are poor models for AAV-mediated gene transfer, so the data was segregated between male and female mice. As shown in FIG. 2A, mice receiving C5Ab04 packaged in either the AAVHSC13 or the AAVHSC17 capsid demonstrated elevated levels of anti-C5 antibodies over time. FIG. 2B shows the results in FIG. 2A with the Y-axis in a logarithmic scale. In FIGS. 2A-2B, n=2-3 mice per group.

In a second experiment, mice received vector C5Ab02 packaged in the AAVHSC17 capsid at a dose of 1e13 vgs/kg. Data for male and female mice were segregated and multiple serum samples were taken over a period of 16 weeks. The serum samples were tested for human IgG concentration (μg/mL) as a readout of anti-C5 antibody levels. As shown in FIG. 2C, mice receiving vector C5Ab02 packaged in the AAVHSC17 capsid demonstrated elevated levels of anti-C5 antibodies over time. FIG. 2D shows the results in FIG. 2C with the Y-axis in a logarithmic scale. In FIGS. 2C-2D, n=3 mice per group.

In a third experiment, mice received vectors C5Ab02, C5Ab03, or C5Ab04, each packaged in the AAVHSC15 or the AAVHSC17 capsid at a dose of 1e13 vgs/kg. Data for male mice is shown and multiple serum samples were taken over a period of 16 weeks. The serum samples were tested for human IgG concentration (μg/mL) as a readout of anti-C5 antibody levels. As shown in FIG. 2E, mice receiving any one of vectors C5Ab02, C5Ab03, or C5Ab04, packaged in either the AAVHSC15 or the AAVHSC17 capsid, demonstrated elevated levels of anti-C5 antibodies over time. FIGS. 2F and 2G (Y-axis in a logarithmic scale) show the results in FIG. 2E presented in line graph format. In FIGS. 2E-2G, n=3 male mice per group.

In a fourth experiment, mice received vector C5Ab04 packaged in the AAVHSC17 capsid at 5 doses, Sell vgs/kg, 5e12 vgs/kg, 1.4e13 vgs/kg, 4.4e13 vgs/kg, and 1.8e14 vgs/kg. Data for male mice is shown and multiple serum samples were taken over a period of 13 weeks. The serum samples were tested for human IgG concentration (μg/mL) as a readout of anti-C5 antibody levels. As shown in the dose response data of FIG. 2H, mice receiving vector C5Ab04 packaged in the AAVHSC17 capsid demonstrated elevated levels of anti-C5 antibodies over time. Moreover, increasing doses of AAVHSC17 lead to corresponding increases in the concentration of anti-C5 antibodies, with doses 1.4e13 vgs/kg, 4.4e13 vgs/kg, and 1.8e14 vgs/kg achieving milligram concentrations of the antibody. FIG. 2I shows the results in FIG. 2H presented in line graph format with the Y-axis in a logarithmic scale. In FIGS. 2E-21, n=3 male mice per group.

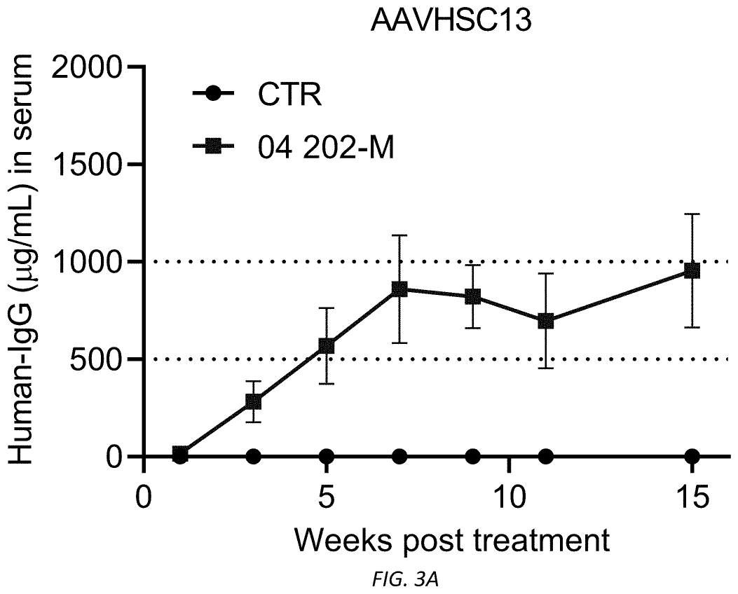

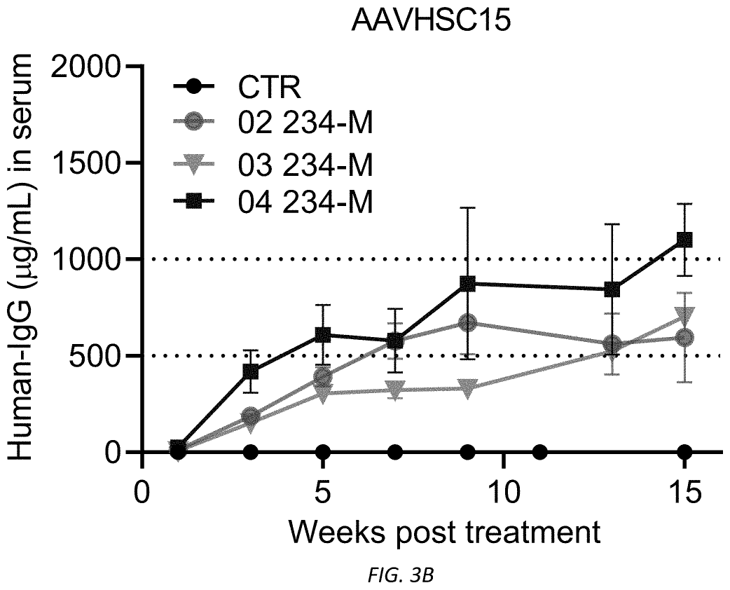

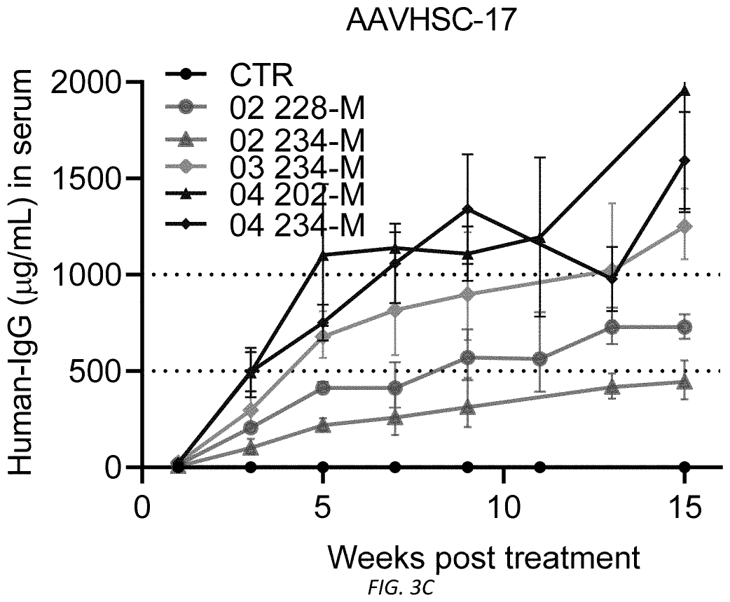

The data above was reorganized to compare the efficacy of each vector packaged in each AAVHSC13, AAVHSC15, or AAVHSC17. As shown in FIGS. 3A-3C, C5Ab04 consistently produced higher concentrations of the anti-C5 antibody over time. Moreover, the AAVHSC17 capsid consistently produced higher concentrations of the anti-C5 antibody over time. The combination of C5Ab04 packaged in the AAVHSC17 capsid produced the highest concentrations of the anti-C5 antibody, at approximately 2000 μg/mL, 15 weeks post-delivery to mice.

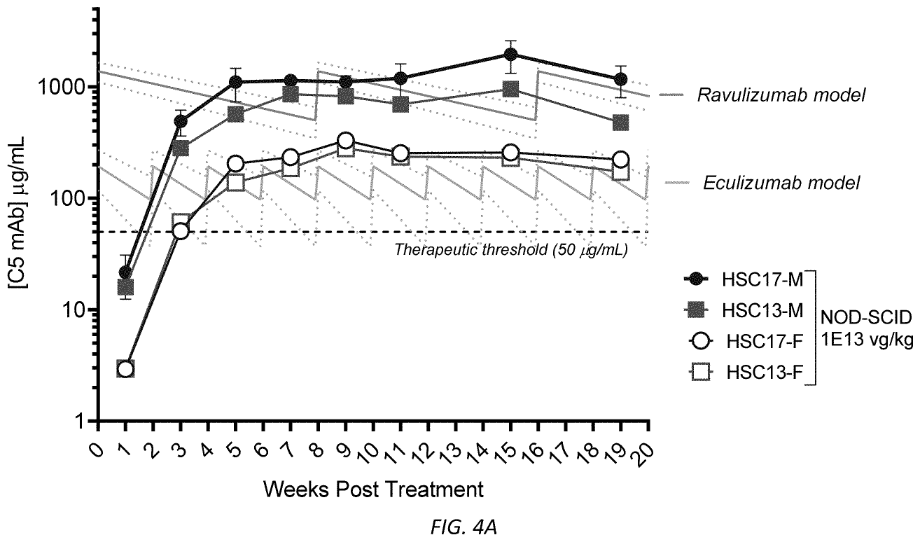

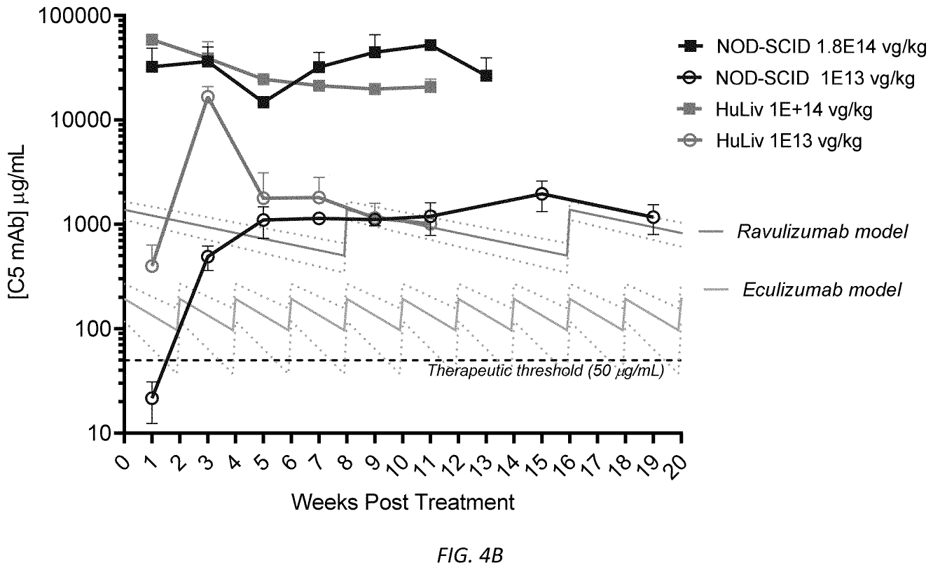

The data above was also used to predict if the anti-C5 antibody concentrations achieved are consistent with known therapeutic anti-C5 antibodies that are administered directly to patients (i.e., not expressed from a vector in the patient). The pharmacokinetics of commercially available eculizumab and ravulizumab were modeled based on Cₘₐₓ, C_{trough}, and dosing schedule data for the treatment of PNH. The standard deviation ranges were based on the coefficient of variation and number of patients used for these studies (dotted lines in FIGS. 4A and 4B). This modeled data was aligned with data from the above in vivo mouse experiments for the AAVHSC13 and AAVHSC17 packaged vectors from the first experiment. The comparison reveals that the therapeutic approach described herein can achieve clinically effective levels of known anti-C5 antibodies, ravulizumab and eculizumab (FIG. 4A). The modeled data was also aligned with data from the above in vivo mouse experiments (first experiment: “NOD-SCID, 1E+13 vg/kg”; and fourth experiment: “NOD-SCID 1.8E+14 vg/kg”), as well as data from the HuLiv mouse experiments below (Example 4), for C5Ab04 packaged in the AAVHSC17 capsid (FIG. 4B).

### Example 3: Ex Vivo Analysis of Anti-C5 Antibodies Expressed in a Mouse Model

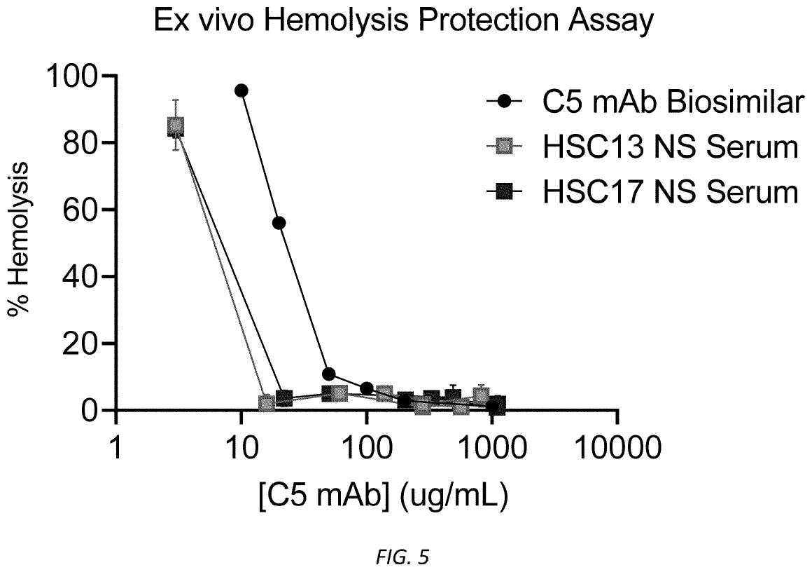

Paroxysmal nocturnal hemoglobinuria (PNH) is characterized by destruction of red blood cells (hemolytic anemia), blood clots (thrombosis), and impaired bone marrow function. To determine if the expressed anti-C5 antibodies are effective at reducing hemolytic anemia, a PNH ex vivo hemolysis assay was performed. Human serum, containing human C5, was mixed with serum obtained from the NOD SCID mice treated with AAVHSC17-packaged C5Ab04, and activated sheep red blood cells (RBCs). The % hemolysis was compared against a control anti-C5 biosimilar antibody. As shown in FIG. 5, the serum obtained from the NOD SCID mice treated with either AAVHSC13-packaged C5Ab04 or AAVHSC17-packaged C5Ab04 was capable of inhibiting hemolysis to a greater extent than the biosimilar control anti-C5 antibody.

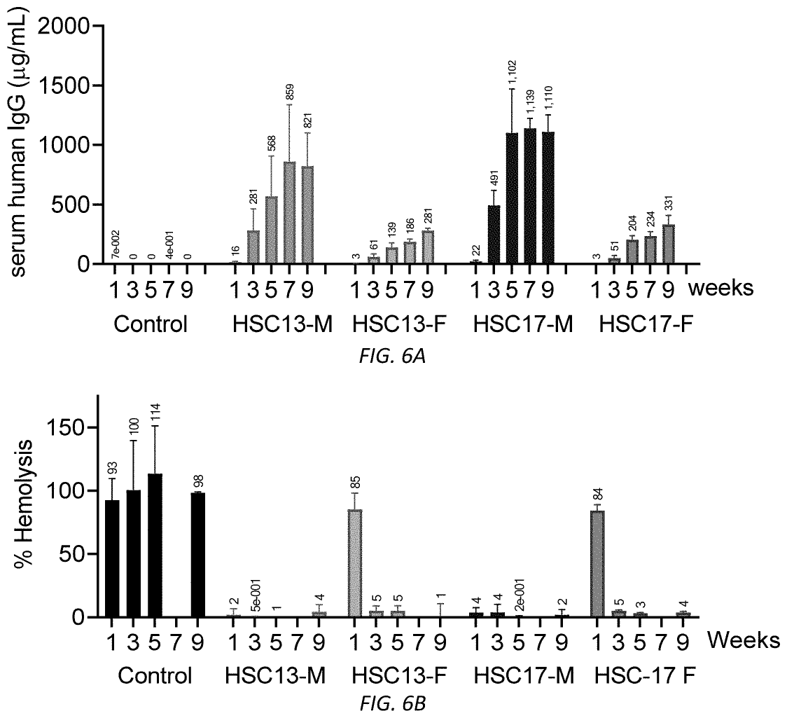

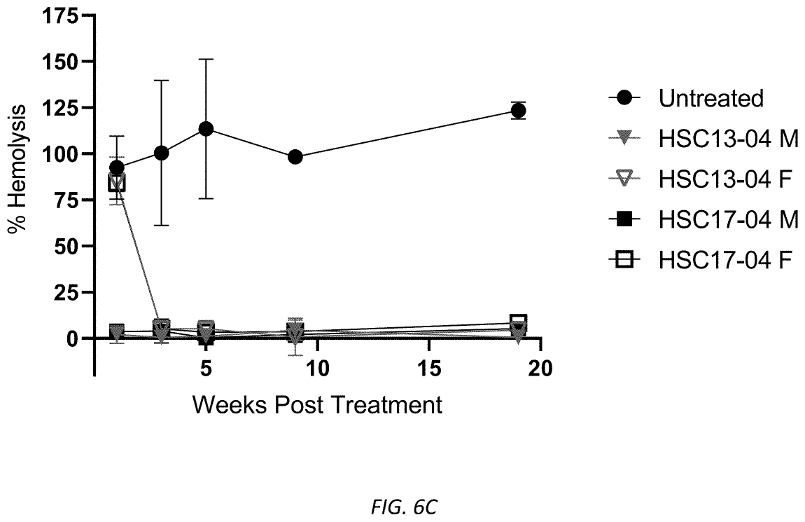

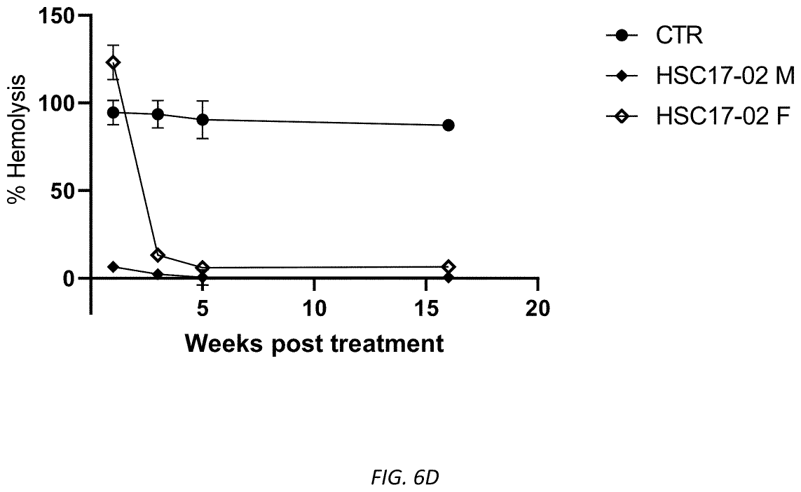

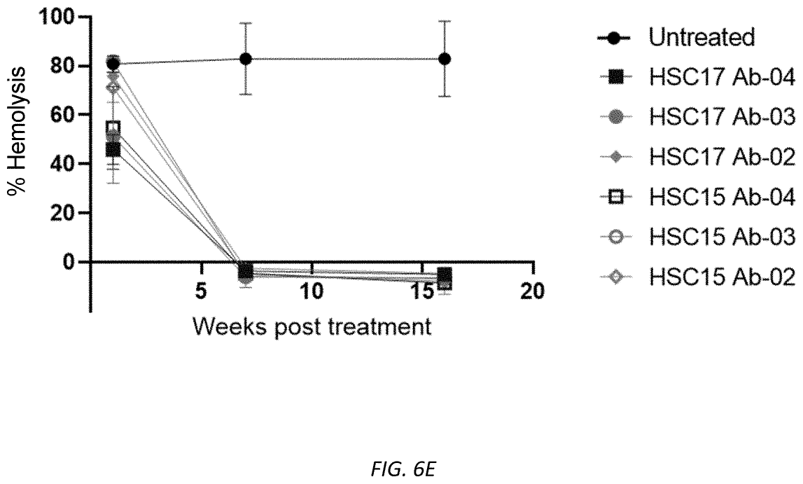

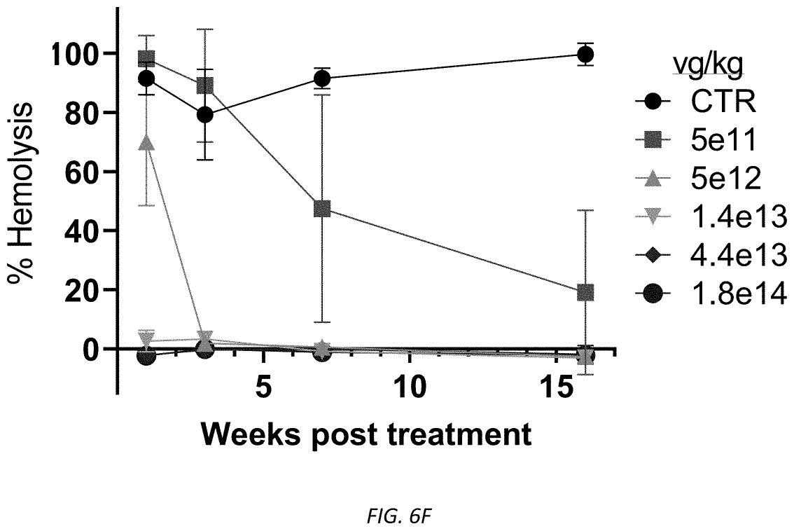

A similar ex vivo hemolysis assay was performed, comparing the serum obtained from the NOD SCID mice treated with either AAVHSC13-packaged C5Ab04 or AAVHSC17-packaged C5Ab04 at a dose of 1e13 vg/kg with negative control mouse serum (from experiment in FIG. 2A). Data for male and female mice were segregated and multiple serum samples were taken over a period of 9 weeks post-treatment. Human IgG concentration (μg/mL) was measured in serum samples as a readout of anti-C5 antibody levels, and demonstrated elevated levels of anti-C5 antibodies over time in mice treated with C5Ab04 packaged in either the AAVHSC13 or the AAVHSC17 capsid (FIG. 6A). Data presented in FIG. 6A is a subset of data presented in FIGS. 2A and 2B. Serum obtained from the mice treated with C5Ab04 packaged in either the AAVHSC13 or the AAVHSC17 capsid was capable of inhibiting hemolysis (FIG. 6B). FIG. 6C shows the results in FIG. 6B with % hemolysis determined from serum samples obtained out to 19 weeks post-administration, and presented in a line graph. In FIGS. 6A-6C, n=3 mice per group.

In a second experiment, an ex vivo hemolysis assay was performed, comparing the serum obtained from NOD SCID mice treated with AAVHSC17-packaged C5Ab02 at a dose of 1e13 vgs/kg, with negative control mouse serum (as in FIG. 2C). Data for male and female mice were segregated and multiple serum samples were taken over a period of 16 weeks post-treatment. As shown in FIG. 6D, serum obtained from the treated NOD SCID mice were capable of inhibiting hemolysis. In FIG. 6D, n=3 mice per group.

In a third experiment, an ex vivo hemolysis assay was performed, comparing the serum obtained from male NOD SCID mice treated with AAVHSC15 or AAVHSC17-packaged C5Ab02, C5Ab03, or C5Ab04, at a dose of 1e13 vgs/kg, with negative control mouse serum (as in FIG. 2E). Multiple serum samples were taken over a period of 16 weeks post-treatment. As shown in FIG. 6E, serum obtained from the treated NOD SCID mice were capable of inhibiting hemolysis. In FIG. 6E, n=3 male mice per group.

In a fourth experiment, an ex vivo hemolysis assay was performed, comparing the serum obtained from male NOD SCID mice treated with AAVHSC17-packaged C5Ab04 at a dose of Sell vgs/kg, 5e12 vgs/kg, 1.4e13 vgs/kg, 4.4e13 vgs/kg, or 1.8e14 vgs/kg, with negative control mouse serum (as in FIG. 2H). Multiple serum samples were taken over a period of 16 weeks post-treatment. As shown in FIG. 6F, serum obtained from the treated NOD SCID mice were capable of inhibiting hemolysis in an rAAV-dose-dependent manner. In FIG. 6F, n=3 male mice per group.

Hemolysis Assay Protocol

The above referenced hemolysis assay was performed using the following protocol. In a 96-well V-bottom plate, Gelatin Veronal buffer (GVBS, Sigma, Cat#G6514) was mixed with mouse serum in each well (with and without EDTA). 10% Normal Human Serum (NHS, Sigma, Cat#H4522) was then added to all wells. The plate was then incubated at 37° C. for 30 minutes. 1 mL of antibody-sensitized sheep erythrocytes (Complement Technology, Inc., Catalog Numbers: B200, B201 and B202) were then added to each well and shaken for about 30 minutes. The plate was then centrifuged at 1000g for 5 minutes. The supernatant was moved to a new plate and read at 540 nm and 615 nm. The 615-nm value was then subtracted from 540 nm value to obtain the final reading. In all reported % hemolysis values, % hemolysis of all samples, including the formulation buffer-only or AAVHSC-treated samples, is reported after normalizing with 100% red blood cell lysis control.

### Example 4: Generation and Characterization of the HuLiv Mouse Model

To evaluate the functional activity and durability of the therapeutic approach described herein Fah^{-/-} Rag2^{-/-} Il2rg^{-/-} mice on the C57Bl/6 background, commonly referred to as the FRG® Knockout mice, were used as a model for liver humanization. The mice were immunodeficient and lacked the tyrosine catabolic enzyme fumarylacetoacetate hydrolase (Fah). Ablation of mouse hepatocytes was induced by the withdrawal of the protective drug 2-(2-nitro-4-trifluoromethylbenzoyl)-1,3-cyclohexanedione (NTBC). The mice were then engrafted with human hepatocytes, and a urokinase-expressing adenovirus was administered to enhance repopulation of the human hepatocytes. Engraftment was sustained over the life of the animal with an appropriate regimen of CuRx™ Nitisinone (20-0026) and prophylactic treatment of SMX/TMP antibiotics (20-0037). Resulting HuLiv mouse livers were repopulated with >70% human hepatocytes. These HuLiv mice are described further in Azuma et al. (Nature Biotechnology. 25(8): 903-910 (2007)).

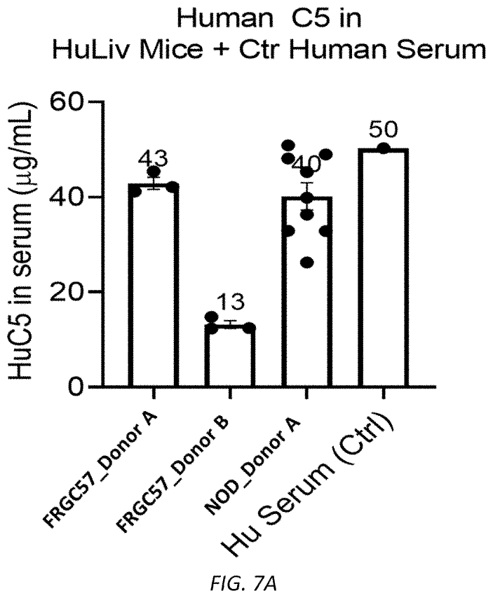

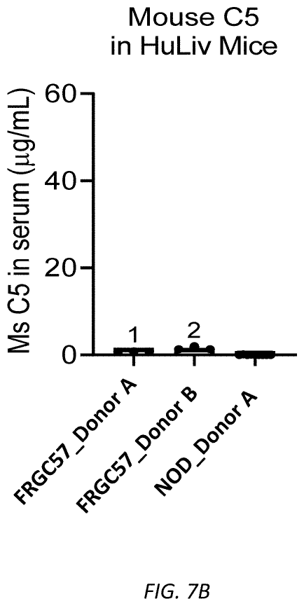

As shown in FIG. 7A and FIG. 7B, the HuLiv mouse produces human C5 at levels comparable to human serum while producing less mouse C5. This HuLiv model allows for the examination of anti-C5 antibody expression by human hepatocytes in vivo, and anti-C5 antibody durability in the presence of human C5.

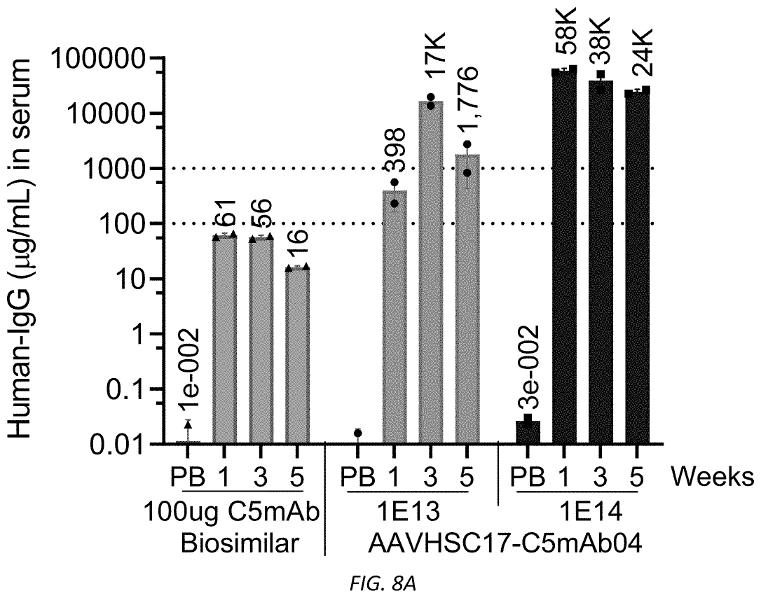

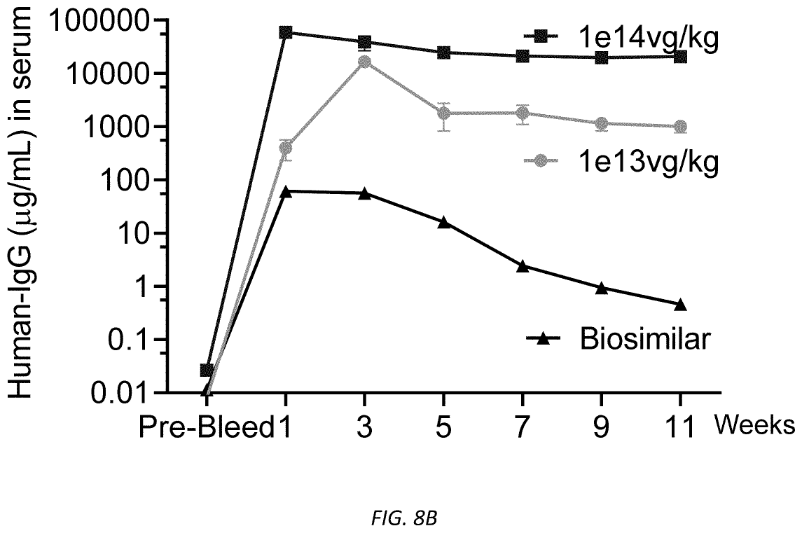

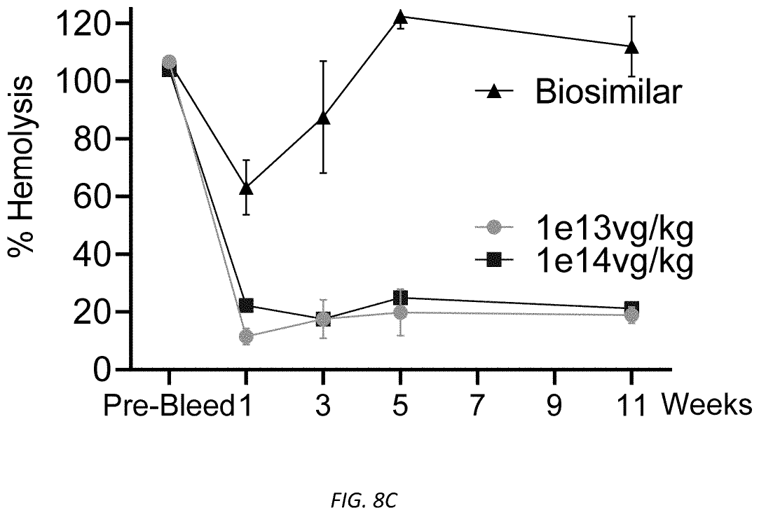

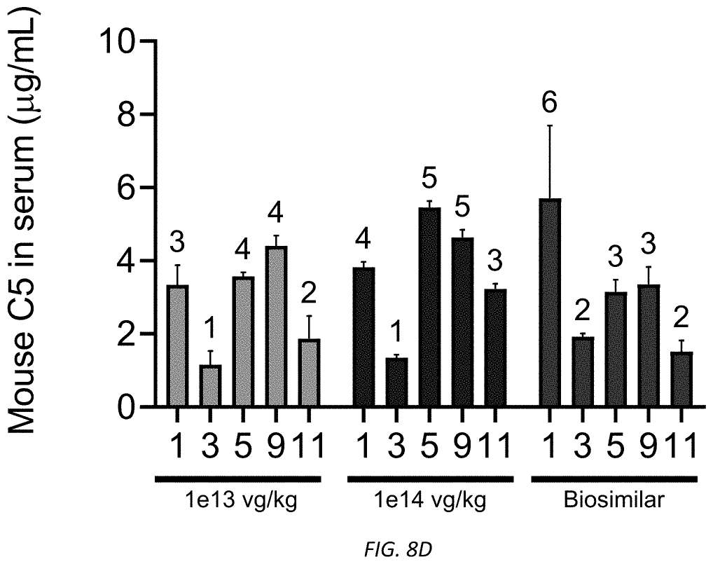

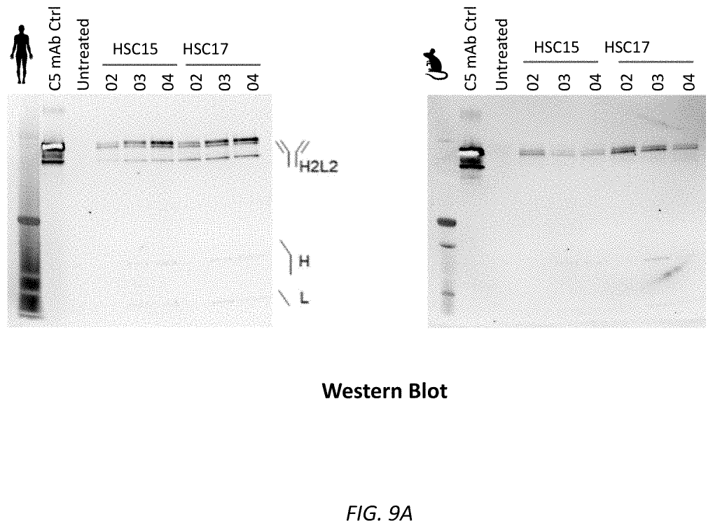

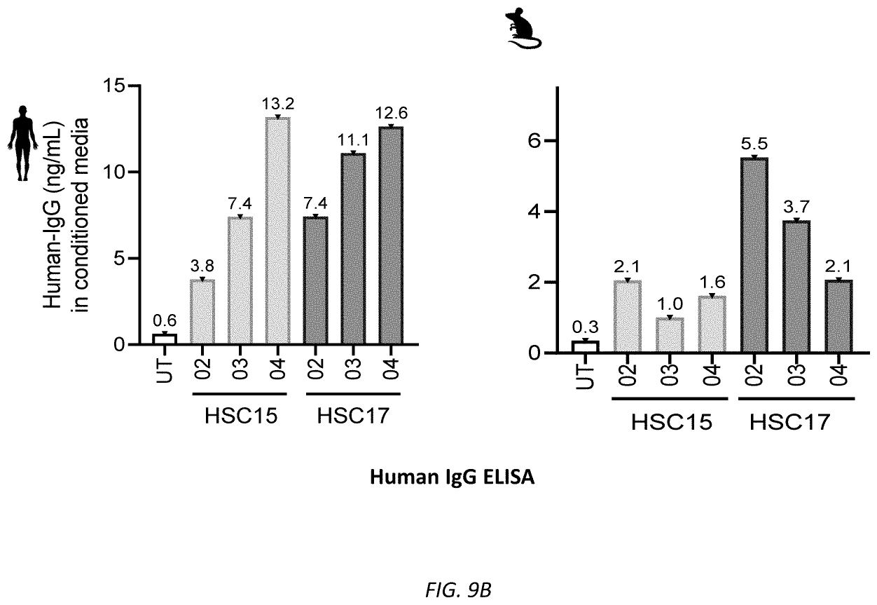

HuLiv mice (n=2) were administered a single dose of 100 μg of a control anti-C5 biosimilar antibody or administered C5Ab04 packaged in the AAVHSC17 capsid at a dose of 1e13 vgs/kg or 1e14 vgs/kg. Serum antibody concentration was determined at week 1, 3, 5, 7, 9, and 11. As shown in FIG. 8A for weeks 1, 3, and 5, administration of C5Ab04 packaged in the AAVHSC17 capsid to the HuLiv mice led to substantially higher concentrations of the anti-C5 antibody compared to direct administration of the control anti-C5 antibody. FIG. 8B shows the results in FIG. 8A with serum antibody concentration determined out to week 11 post-administration, and presented in a line graph with the Y-axis in a logarithmic scale. An ex vivo hemolysis assay was performed using the same methodology as described above, comparing the serum obtained from the HuLiv mice treated with C5Ab04 packaged in the AAVHSC17 capsid at a dose of 1e13 vgs/kg or 1e14 vgs/kg, with the serum obtained from a HuLiv mouse directly administered a control anti-C5 antibody (biosimilar). Serum samples were taken over a period of 11 weeks post-treatment. As shown in FIG. 8C, serum obtained from the treated HuLiv mice were capable of inhibiting hemolysis. The mice treated with C5Ab04 packaged in AAVHSC17 capsid at a dose of 1e13 vgs/kg or 1e14 vgs/kg showed about 80% protection from hemolysis in the ex vivo hemolysis assay. Background residual hemolysis of up to about 20% may be explained by the presence of mouse complement proteins made by a residual population of C57Bl/6 hepatocytes in HuLiv mice. FIG. 8D shows the level of mouse C5 detected via an enzyme-linked immunosorbent assay in serum obtained from the treated HuLiv mice.

### Example 5: Primary Hepatocyte Screening Assay

In order to rapidly test optimized rAAV vectors, a cell line-based assay was developed to assess antibody production and secretion. Human primary hepatocytes were selected as the closest match to the in vivo experiments. Plateable human hepatocytes (Cat. # HUCPG) and plateable C57BL/6 mouse hepatocytes (Cat. # MBCP01) from Lonza were used.

Approximately 500,000 human hepatocytes or 250,000 mouse hepatocytes were plated, followed by incubation with approximately 300,000 MOI of AAVHSC15 or AAVHSC17 packaged with C5Ab02, C5Ab03, or C5Ab04. Culture media was then collected on day 7 after viral addition and analyzed by western blot and human IgG ELISA. As shown in FIG. 9A and FIG. 9B, abundant levels of the anti-C5 antibodies were detected.

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes. Other embodiments are within the following claims.

1. A recombinant adeno-associated virus (rAAV) genome comprising:
(a) a first expression cassette comprising, from 5′ to 3′,
a first liver-specific transcriptional regulatory element,
a first coding sequence encoding a first polypeptide comprising an antibody heavy chain operably linked to a first signal sequence, and
a first polyadenylation sequence; and
(b) a second expression cassette comprising, from 5′ to 3′,
a second liver-specific transcriptional regulatory element,
a second coding sequence encoding a second polypeptide comprising an antibody light chain operably linked to a second signal sequence, and
a second polyadenylation sequence,
wherein expression of the first and second coding sequences produces an antibody comprising the antibody heavy chain and the antibody light chain.

2. The rAAV genome of claim 1, wherein the first and/or second transcriptional regulatory element comprise a promoter element selected from the group consisting of human albumin promoter, a human transthyretin (TTR) promoter, a human thyroxine binding globulin (TBG) promoter, a human ApoH promoter, a human SERPINA1 (hAAT) promoter, and a hepatic specific regulatory module thereof, such as a human ApoE/C-I hepatic control region (HCR) 1 or 2, optionally wherein the promoter element comprises a nucleic acid sequence at least 90% identical to a sequence selected from the group consisting of SEQ ID NO: 25, 27, 66, 67, 68, 69, 116, and 117.

3-9. (canceled)

10. The rAAV genome of claim 1, wherein the first and/or second expression cassette further comprises an intron element positioned 5′ to the first and/or second coding sequence and 3′ to the transcriptional regulatory element, optionally wherein:
the intron element is an exogenous intron element;
the intron element is an SV40 intron element or a minute virus of mouse (MVM) intron element; and/or
the intron element comprises a nucleic acid sequence at least 90% identical to SEQ ID NO: 29 or 30.

11-17. (canceled)

18. The rAAV genome of claim 1, wherein the first and second transcriptional regulatory element are identical.

19. The rAAV genome of claim 1, wherein:
the first transcriptional regulatory element comprises an HCR 1 element, a hAAT promoter, and/or an SV40 intron element, optionally wherein the first transcriptional regulatory element comprises a nucleic acid sequence at least 90% identical to SEQ ID NO: 50; and
the second transcriptional regulatory element comprises a SERPINA1 hepatic specific regulatory module, a TTR promoter, and/or an MVM intron element, optionally wherein the second transcriptional regulatory element comprises a nucleic acid sequence at least 90% identical to SEQ ID NO: 43.

20. (canceled)

21. The rAAV genome of claim 1, wherein the first and/or second expression cassette further comprises a polyadenylation sequence 3′ to the first and/or second coding sequence, optionally wherein the polyadenylation sequence is an exogenous polyadenylation sequence, optionally wherein:
the exogenous polyadenylation sequence is an SV40 polyadenylation sequence, or a bovine growth hormone (BGH) polyadenylation sequence;
the polyadenylation sequence comprises a nucleic acid sequence at least 90% identical to SEQ ID NO: 31 or 33;
the first and second expression cassette comprise identical polyadenylation sequences;
the first expression cassette comprises the SV40 polyadenylation sequence;
the second expression cassette comprises the BGH polyadenylation sequence; and/or
the first polyadenylation sequence comprises the nucleic acid sequence of SEQ ID NO: 31 and the second polyadenylation sequence comprises the nucleic acid sequence of SEQ ID NO: 33.

22-32. (canceled)

33. The rAAV genome of claim 1, wherein the first and second expression cassettes are in the same orientation in the rAAV genome or the first and second expression cassettes are in opposite orientations in the rAAV genome, optionally with the first and second polyadenylation sequences distally positioned in the rAAV genome.

34-35. (canceled)

36. The rAAV genome of claim 1, wherein the rAAV genome further comprises a stuffer sequence interposed between the first and second transcriptional regulatory elements, optionally wherein the stuffer sequence comprises a beta globin polyadenylation sequence, optionally a nucleic acid sequence at least 90% identical to SEQ ID NO: 51.

37-38. (canceled)

39. The rAAV genome of claim 1, wherein the rAAV genome comprises from 5′ to 3′: (a) the first polyadenylation sequence comprising the nucleic acid sequence of SEQ ID NO: 33; (b) the first coding sequence; (c) the first liver-specific transcriptional regulatory element comprising the nucleic acid sequence of SEQ ID NO: 27; (d) a stuffer sequence comprising the nucleic acid sequence of SEQ ID NO: 51; (e) the second liver-specific transcriptional regulatory element comprising the nucleic acid sequence of SEQ ID NO: 67; (f) the second coding sequence; and (g) the second transcriptional polyadenylation sequence comprising the nucleic acid sequence of SEQ ID NO: 31.

40. The rAAV genome of claim 1, wherein the rAAV genome comprises from 5′ to 3′: the reverse complement of the first expression cassette; a stuffer sequence; and the second expression cassette, optionally wherein:
(a) the first expression cassette comprises, from 5′ to 3′: a nucleotide sequence at least 90% identical to the nucleotide sequence set forth in SEQ ID NO: 27, the first coding sequence, a nucleotide sequence at least 90% identical to the nucleotide sequence set forth in SEQ ID NO: 33; (b) the stuffer sequence comprising a nucleotide sequence at least 90% identical to the nucleotide sequence set forth in SEQ ID NO: 51 or the reverse complement thereof; and (c) the second expression cassette comprising, from 5′ to 3′, a nucleotide sequence at least 90% identical to the nucleotide sequence set forth in SEQ ID NO: 67, the second coding sequence, a nucleotide sequence at least 90% identical to the nucleotide sequence set forth in SEQ ID NO: 31;
(a) the first expression cassette comprises, from 5′ to 3′: a nucleotide sequence at least 90% identical to the nucleotide sequence set forth in SEQ ID NO: 67, the first coding sequence, a nucleotide sequence at least 90% identical to the nucleotide sequence set forth in SEQ ID NO: 31; (b) the stuffer sequence comprising a nucleotide sequence at least 90% identical to the nucleotide sequence set forth in SEQ ID NO: 51 or the reverse complement thereof; and (c) the second expression cassette comprising, from 5′ to 3′, a nucleotide sequence at least 90% identical to the nucleotide sequence set forth in SEQ ID NO: 27, the second coding sequence, a nucleotide sequence at least 90% identical to the nucleotide sequence set forth in SEQ ID NO: 33;
(a) the first expression cassette comprises, from 5′ to 3′: a nucleotide sequence at least 90% identical to the nucleotide sequence set forth in SEQ ID NO: 25, a nucleotide sequence at least 90% identical to the nucleotide sequence set forth in SEQ ID NO: 26, the first coding sequence, the first polyadenylation sequence; (b) the stuffer sequence comprising a nucleotide sequence at least 90% identical to the nucleotide sequence set forth in SEQ ID NO: 51 or the reverse complement thereof; and (c) the second expression cassette comprising, from 5′ to 3′, a nucleotide sequence at least 90% identical to the nucleotide sequence set forth in SEQ ID NO: 119, a nucleotide sequence at least 90% identical to the nucleotide sequence set forth in SEQ ID NO: 45, the second coding sequence, a nucleotide sequence at least 90% identical to the nucleotide sequence set forth in SEQ ID NO: 31;
(a) the first expression cassette comprises, from 5′ to 3′: a nucleotide sequence at least 90% identical to the nucleotide sequence set forth in SEQ ID NO: 119, a nucleotide sequence at least 90% identical to the nucleotide sequence set forth in SEQ ID NO: 45, the first coding sequence, a nucleotide sequence at least 90% identical to the nucleotide sequence set forth in SEQ ID NO: 31; (b) the stuffer sequence comprising a nucleotide sequence at least 90% identical to the nucleotide sequence set forth in SEQ ID NO: 51 or the reverse complement thereof; and (c) the second expression cassette comprising, from 5′ to 3′, a nucleotide sequence at least 90% identical to the nucleotide sequence set forth in SEQ ID NO: 25, a nucleotide sequence at least 90% identical to the nucleotide sequence set forth in SEQ ID NO: 26, the second coding sequence, the first polyadenylation sequence;
(a) the first expression cassette comprises, from 5′ to 3′: the nucleotide sequence set forth in SEQ ID NO: 27, the first coding sequence, the nucleotide sequence set forth in SEQ ID NO: 33; (b) the stuffer sequence comprising the nucleotide sequence set forth in SEQ ID NO: 51 or the reverse complement thereof and (c) the second expression cassette comprising, from 5′ to 3′, the nucleotide sequence set forth in SEQ ID NO: 67, the second coding sequence, the nucleotide sequence set forth in SEQ ID NO: 31;
(a) the first expression cassette comprises, from 5′ to 3′: the nucleotide sequence set forth in SEQ ID NO: 67, the first coding sequence, the nucleotide sequence set forth in SEQ ID NO: 31; (b) the stuffer sequence comprising the nucleotide sequence set forth in SEQ ID NO: 51 or the reverse complement thereof and (c) the second expression cassette comprising, from 5′ to 3′, the nucleotide sequence set forth in SEQ ID NO: 27, the second coding sequence, the nucleotide sequence set forth in SEQ ID NO: 33;
(a) the first expression cassette comprises, from 5′ to 3′: the nucleotide sequence set forth in SEQ ID NO: 25, the nucleotide sequence set forth in SEQ ID NO: 26, the first coding sequence, the first polyadenylation sequence; (b) the stuffer sequence comprising the nucleotide sequence set forth in SEQ ID NO: 51 or the reverse complement thereof; and (c) the second expression cassette comprising, from 5′ to 3′, the nucleotide sequence set forth in SEQ ID NO: 119, the nucleotide sequence set forth in SEQ ID NO: 45, the second coding sequence, the nucleotide sequence set forth in SEQ ID NO: 31; or
(a) the first expression cassette comprises, from 5′ to 3′: the nucleotide sequence set forth in SEQ ID NO: 119, the nucleotide sequence set forth in SEQ ID NO: 45, the first coding sequence, the nucleotide sequence set forth in SEQ ID NO: 31; (b) the stuffer sequence comprising the nucleotide sequence set forth in SEQ ID NO: 51 or the reverse complement thereof; and (c) the second expression cassette comprising, from 5′ to 3′, the nucleotide sequence set forth in SEQ ID NO: 25, the nucleotide sequence set forth in SEQ ID NO: 26, the second coding sequence, the first polyadenylation sequence.

41-48. (canceled)

49. An rAAV genome comprising a bicistronic expression cassette comprising, from 5′ to 3′:
(a) a liver-specific transcriptional regulatory element; a first coding sequence encoding a first polypeptide comprising an antibody heavy chain operably linked to a first signal sequence; a ribosomal skipping sequence encoding a ribosomal skipping peptide; a second coding sequence encoding a second polypeptide comprising an antibody light chain operably linked to a second signal sequence; and a polyadenylation sequence; or
(b) a liver-specific transcriptional regulatory element; a second coding sequence encoding a second polypeptide comprising an antibody light chain operably linked to a second signal sequence; a ribosomal skipping sequence encoding a ribosomal skipping peptide; a first coding sequence encoding a first polypeptide comprising an antibody heavy chain operably linked to a first signal sequence; and a polyadenylation sequence,
wherein expression of the bicistronic expression cassette produces an antibody comprising the antibody heavy chain and the antibody light chain.

50. The rAAV genome of claim 49, wherein transcriptional regulatory element comprises a promoter element selected from the group consisting of human albumin promoter, a human transthyretin (TTR) promoter, the human thyroxine binding globulin (TBG) promoter, a human ApoH promoter, a human SERPINA1 (hAAT) promoter, and a hepatic specific regulatory module thereof, such as a human ApoE/C-I hepatic control region (HCR) 1 or 2, optionally wherein the promoter element comprises a nucleic acid sequence at least 90% identical to a sequence selected from the group consisting of SEQ ID NO: 25, 27, 66, 67, 68, 69, 116, and 117.

51-57. (canceled)

58. The rAAV genome of claim 49, wherein the bicistronic expression cassette further comprises an intron element positioned 5′ to the first and/or second coding sequence and 3′ to the transcriptional regulatory element, optionally wherein:
the intron element is an exogenous intron element;
the intron element is an SV40 intron element or a minute virus of mouse (MVM) intron element; and/or
the intron element comprises a nucleic acid sequence at least 90% identical to SEQ ID NO: 29 or 30.

59-65. (canceled)

66. The rAAV genome of claim 49, wherein the transcriptional regulatory element comprises:
(a) an HCR 1 element, a hAAT promoter, and an SV40 intron element;
(b) a SERPINA1 hepatic specific regulatory module, a TTR promoter, and an MVM intron element; and/or
(c) the nucleic acid sequence of SEQ ID NO: 43 or 50.

67. (canceled)

68. The rAAV genome of claim 49, wherein the polyadenylation sequence is an exogenous polyadenylation sequence, optionally wherein:
the exogenous polyadenylation sequence is an SV40 polyadenylation sequence, or a bovine growth hormone (BGH) polyadenylation sequence; and/or
the polyadenylation sequence comprises a nucleic acid sequence at least 90% identical to SEQ ID NO: 31 or 33.

69-74. (canceled)

75. The rAAV genome of claim 1, wherein:
the first and/or second signal sequence is a naturally occurring signal sequence;
the first and/or second signal sequence is an antibody signal sequence, optionally a human IgG2 or IgK signal sequence;
the first and/or second signal sequence is a non-naturally occurring signal sequence;
the first and/or second signal sequence comprises the amino acid sequence of SEQ ID NO: 80; or
the first and/or second signal sequence comprises the amino acid sequence of SEQ ID NO: 81,
optionally wherein:
the first signal sequence comprises the amino acid sequence of SEQ ID NO: 80, and the second signal sequence comprises the amino acid sequence of SEQ ID NO: 81;
the first and/or second coding sequence comprises any one of the nucleic acid sequences set forth in SEQ ID NOs: 23, 96, 102, or 108;
the first and/or second coding sequence comprises any one of the nucleic acid sequences set forth in SEQ ID NOs: 24, 99, 105, 111, or 130; and/or
the first coding sequence comprises any one of the nucleic acid sequences set forth in SEQ ID NOs: 23, 96, 102, or 108 and the second coding sequence comprises any one of the nucleic acid sequences set forth in SEQ ID NOs: 24, 99, 105, 111, or 130.

76-83. (canceled)

84. The rAAV genome of claim 1, wherein the antibody specifically binds to complement C5, optionally wherein:
the antibody heavy chain comprises the amino acid sequence of SEQ ID NO: 64 or 82;
the antibody light chain comprises the amino acid sequence of SEQ ID NO: 77;
first and/or second coding sequence has been optimized for expression in human cells;
the first coding sequence comprises any one of the nucleic acid sequences set forth in SEQ ID NO: 52, 113, 114, or 115;
the first coding sequence comprises any one of the nucleic acid sequences set forth in SEQ ID NOs: 83, 94, 95, 101, or 107;
the second coding sequence comprises any one of the nucleic acid sequences set forth in SEQ ID NOs: 53, 98, 104, 110, or 131;
the first coding sequence comprises a nucleotide sequence selected from the group consisting of SEQ ID NO: 52, 62, 83, 94, 95, 96, 97, 101, 102, 103, 107, 108, 109, 113, 114, and 115, and the second coding sequence comprises the nucleotide sequence set forth in SEQ ID NO: 53;
the first coding sequence comprises a nucleotide sequence selected from the group consisting of SEQ ID NO: 52, 62, 83, 94, 95, 96, 97, 101, 102, 103, 107, 108, 109, 113, 114, and 115, and the second coding sequence comprises the nucleotide sequence set forth in SEQ ID NO: 63;
the first coding sequence comprises a nucleotide sequence selected from the group consisting of SEQ ID NO: 52, 62, 83, 94, 95, 96, 97, 101, 102, 103, 107, 108, 109, 113, 114, and 115, and the second coding sequence comprises the nucleotide sequence set forth in SEQ ID NO: 98;
the first coding sequence comprises a nucleotide sequence selected from the group consisting of SEQ ID NO: 52, 62, 83, 94, 95, 96, 97, 101, 102, 103, 107, 108, 109, 113, 114, and 115, and the second coding sequence comprises the nucleotide sequence set forth in SEQ ID NO: 99;
the first coding sequence comprises a nucleotide sequence selected from the group consisting of SEQ ID NO: 52, 62, 83, 94, 95, 96, 97, 101, 102, 103, 107, 108, 109, 113, 114, and 115, and the second coding sequence comprises the nucleotide sequence set forth in SEQ ID NO: 100;
the first coding sequence comprises a nucleotide sequence selected from the group consisting of SEQ ID NO: 52, 62, 83, 94, 95, 96, 97, 101, 102, 103, 107, 108, 109, 113, 114, and 115, and the second coding sequence comprises the nucleotide sequence set forth in SEQ ID NO: 104;
the first coding sequence comprises a nucleotide sequence selected from the group consisting of SEQ ID NO: 52, 62, 83, 94, 95, 96, 97, 101, 102, 103, 107, 108, 109, 113, 114, and 115, and the second coding sequence comprises the nucleotide sequence set forth in SEQ ID NO: 105;
the first coding sequence comprises a nucleotide sequence selected from the group consisting of SEQ ID NO: 52, 62, 83, 94, 95, 96, 97, 101, 102, 103, 107, 108, 109, 113, 114, and 115, and the second coding sequence comprises the nucleotide sequence set forth in SEQ ID NO: 106;
the first coding sequence comprises a nucleotide sequence selected from the group consisting of SEQ ID NO: 52, 62, 83, 94, 95, 96, 97, 101, 102, 103, 107, 108, 109, 113, 114, and 115, and the second coding sequence comprises the nucleotide sequence set forth in SEQ ID NO: 110;
the first coding sequence comprises a nucleotide sequence selected from the group consisting of SEQ ID NO: 52, 62, 83, 94, 95, 96, 97, 101, 102, 103, 107, 108, 109, 113, 114, and 115, and the second coding sequence comprises the nucleotide sequence set forth in SEQ ID NO: 111; and/or
the first coding sequence comprises a nucleotide sequence selected from the group consisting of SEQ ID NO: 52, 62, 83, 94, 95, 96, 97, 101, 102, 103, 107, 108, 109, 113, 114, and 115, and the second coding sequence comprises the nucleotide sequence set forth in SEQ ID NO: 112.

103. The rAAV genome of claim 1, wherein the rAAV genome is a single stranded rAAV genome or a self-complementary rAAV genome, optionally wherein the rAAV genome comprises the nucleic acid sequence of SEQ ID NO: 84, 85, 86, or 87.

104-108. (canceled)

109. The rAAV genome of claim 1, wherein the rAAV genome further comprises a 5′ inverted terminal repeat (5′ ITR) nucleotide sequence 5′ to the first polyadenylation sequence, and a 3′ inverted terminal repeat (3′ ITR) nucleotide sequence 3′ to the second polyadenylation sequence, optionally wherein:
the 5′ ITR nucleotide sequence is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 14, and/or the 3′ ITR nucleotide sequence is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 18; and/or
the rAAV genome comprises the nucleic acid sequence of SEQ ID NO: 88, 89, 90, or 91.

110-114. (canceled)

115. A recombinant adeno-associated virus (rAAV) comprising:
(a) an AAV capsid comprising an AAV capsid protein; and
(b) an rAAV genome of claim 1.

116. The rAAV of claim 115, wherein:
the AAV capsid protein is selected from the group consisting of AAV1, AAV2, AAV3, AAV4, AAV5, AAV6, AAV7, AAV8, and AAV9;
the AAV capsid protein comprises an amino acid sequence that is at least 95% identical to the amino acid sequence of amino acids 203-736 of SEQ ID NO: 1, 2, 3, 4, 5, 6, 7, 8, 9, 10, 11, 12, 13, 15, 16, or 17, optionally wherein:
the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q, the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G;
(a) the amino acid in the capsid protein corresponding to amino acid 626 of SEQ ID NO: 16 is G, and the amino acid in the capsid protein corresponding to amino acid 718 of SEQ ID NO: 16 is G; (b) the amino acid in the capsid protein corresponding to amino acid 296 of SEQ ID NO: 16 is H, the amino acid in the capsid protein corresponding to amino acid 464 of SEQ ID NO: 16 is N, the amino acid in the capsid protein corresponding to amino acid 505 of SEQ ID NO: 16 is R, and the amino acid in the capsid protein corresponding to amino acid 681 of SEQ ID NO: 16 is M; (c) the amino acid in the capsid protein corresponding to amino acid 505 of SEQ ID NO: 16 is R, and the amino acid in the capsid protein corresponding to amino acid 687 of SEQ ID NO: 16 is R; (d) the amino acid in the capsid protein corresponding to amino acid 346 of SEQ ID NO: 16 is A, and the amino acid in the capsid protein corresponding to amino acid 505 of SEQ ID NO: 16 is R; or (e) the amino acid in the capsid protein corresponding to amino acid 501 of SEQ ID NO: 16 is I, the amino acid in the capsid protein corresponding to amino acid 505 of SEQ ID NO: 16 is R, and the amino acid in the capsid protein corresponding to amino acid 706 of SEQ ID NO: 16 is C; or
the capsid protein comprises the amino acid sequence of amino acids 203-736 of SEQ ID NO: 1, 2, 3, 4, 5, 6, 7, 8, 9, 10, 11, 12, 13, 15, 16, or 17;
the AAV capsid protein comprises an amino acid sequence that is at least 95% identical to the amino acid sequence of amino acids 138-736 of SEQ ID NO: 1, 2, 3, 4, 5, 6, 7, 8, 9, 10, 11, 12, 13, 15, 16, or 17, optionally wherein:
the amino acid in the capsid protein corresponding to amino acid 151 of SEQ ID NO: 16 is R; the amino acid in the capsid protein corresponding to amino acid 160 of SEQ ID NO: 16 is D; the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q, the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G;
(a) the amino acid in the capsid protein corresponding to amino acid 626 of SEQ ID NO: 16 is G, and the amino acid in the capsid protein corresponding to amino acid 718 of SEQ ID NO: 16 is G; (b) the amino acid in the capsid protein corresponding to amino acid 296 of SEQ ID NO: 16 is H, the amino acid in the capsid protein corresponding to amino acid 464 of SEQ ID NO: 16 is N, the amino acid in the capsid protein corresponding to amino acid 505 of SEQ ID NO: 16 is R, and the amino acid in the capsid protein corresponding to amino acid 681 of SEQ ID NO: 16 is M; (c) the amino acid in the capsid protein corresponding to amino acid 505 of SEQ ID NO: 16 is R, and the amino acid in the capsid protein corresponding to amino acid 687 of SEQ ID NO: 16 is R; (d) the amino acid in the capsid protein corresponding to amino acid 346 of SEQ ID NO: 16 is A, and the amino acid in the capsid protein corresponding to amino acid 505 of SEQ ID NO: 16 is R; or (e) the amino acid in the capsid protein corresponding to amino acid 501 of SEQ ID NO: 16 is I, the amino acid in the capsid protein corresponding to amino acid 505 of SEQ ID NO: 16 is R, and the amino acid in the capsid protein corresponding to amino acid 706 of SEQ ID NO: 16 is C; or
the capsid protein comprises the amino acid sequence of amino acids 138-736 of SEQ ID NO: 1, 2, 3, 4, 5, 6, 7, 9, 10, 11, 12, 13, 15, 16, or 17; and/or
the AAV capsid protein comprises an amino acid sequence that is at least 95% identical to the amino acid sequence of amino acids 1-736 of SEQ ID NO: 1, 2, 3, 4, 5, 6, 7, 8, 9, 10, 11, 12, 13, 15, 16, or 17, optionally wherein:
the amino acid in the capsid protein corresponding to amino acid 2 of SEQ ID NO: 16 is T; the amino acid in the capsid protein corresponding to amino acid 65 of SEQ ID NO: 16 is I; the amino acid in the capsid protein corresponding to amino acid 68 of SEQ ID NO: 16 is V; the amino acid in the capsid protein corresponding to amino acid 77 of SEQ ID NO: 16 is R; the amino acid in the capsid protein corresponding to amino acid 119 of SEQ ID NO: 16 is L; the amino acid in the capsid protein corresponding to amino acid 151 of SEQ ID NO: 16 is R; the amino acid in the capsid protein corresponding to amino acid 160 of SEQ ID NO: 16 is D; the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q, the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G;
(a) the amino acid in the capsid protein corresponding to amino acid 2 of SEQ ID NO: 16 is T, and the amino acid in the capsid protein corresponding to amino acid 312 of SEQ ID NO: 16 is Q; (b) the amino acid in the capsid protein corresponding to amino acid 65 of SEQ ID NO: 16 is I, and the amino acid in the capsid protein corresponding to amino acid 626 of SEQ ID NO: 16 is Y; (c) the amino acid in the capsid protein corresponding to amino acid 77 of SEQ ID NO: 16 is R, and the amino acid in the capsid protein corresponding to amino acid 690 of SEQ ID NO: 16 is K; (d) the amino acid in the capsid protein corresponding to amino acid 119 of SEQ ID NO: 16 is L, and the amino acid in the capsid protein corresponding to amino acid 468 of SEQ ID NO: 16 is S; (e) the amino acid in the capsid protein corresponding to amino acid 626 of SEQ ID NO: 16 is G, and the amino acid in the capsid protein corresponding to amino acid 718 of SEQ ID NO: 16 is G; (f) the amino acid in the capsid protein corresponding to amino acid 296 of SEQ ID NO: 16 is H, the amino acid in the capsid protein corresponding to amino acid 464 of SEQ ID NO: 16 is N, the amino acid in the capsid protein corresponding to amino acid 505 of SEQ ID NO: 16 is R, and the amino acid in the capsid protein corresponding to amino acid 681 of SEQ ID NO: 16 is M; (g) the amino acid in the capsid protein corresponding to amino acid 505 of SEQ ID NO: 16 is R, and the amino acid in the capsid protein corresponding to amino acid 687 of SEQ ID NO: 16 is R; (h) the amino acid in the capsid protein corresponding to amino acid 346 of SEQ ID NO: 16 is A, and the amino acid in the capsid protein corresponding to amino acid 505 of SEQ ID NO: 16 is R; or (i) the amino acid in the capsid protein corresponding to amino acid 501 of SEQ ID NO: 16 is I, the amino acid in the capsid protein corresponding to amino acid 505 of SEQ ID NO: 16 is R, and the amino acid in the capsid protein corresponding to amino acid 706 of SEQ ID NO: 16 is C; or
the capsid protein comprises the amino acid sequence of amino acids 1-736 of SEQ ID NO: 1, 2, 3, 4, 5, 6, 7, 8, 9, 10, 11, 12, 13, 15, 16, or 17.

117-128. (canceled)

129. A polynucleotide comprising the nucleic acid sequence set forth in SEQ ID NOs: 85-93.

130. A pharmaceutical composition comprising an rAAV of claim 1, or a polynucleotide comprising the nucleic acid sequence set forth in SEQ ID NOs: 85-93.

131. A packaging system for preparation of an rAAV, wherein the packaging system comprises:
(a) a first nucleotide sequence encoding one or more AAV Rep proteins;
(b) a second nucleotide sequence encoding an AAV capsid protein; and
(c) a third nucleotide sequence comprising an rAAV genome sequence of claim 1.

132-136. (canceled)

137. A method for recombinant preparation of an rAAV, the method comprising introducing the packaging system of claim 131 into a cell under conditions whereby the rAAV is produced.

138-140. (canceled)

141. A method of producing an antibody in a subject, the method comprising administering to the subject the pharmaceutical composition of claim 130, optionally wherein the pharmaceutical composition is administered intravenously.

142. (canceled)

143. A method of treating a complement C5-associated disease in a subject in need thereof, the method comprising administering to the subject an effective amount of the rAAV of claim 1, optionally wherein:
the rAAV is administered intravenously;
the rAAV is comprised within a pharmaceutical composition; and/or
the complement C5-associated disease is selected from the group consisting of geographic atrophy (GA), Guillain-Barré syndrome, myasthenia gravis, systemic lupus erythematous (SLE) nephritis, proliferative nephritis, asthma, rheumatoid arthritis, sepsis, paroxysmal nocturnal hemoglobinuria (PNH), atypical hemolytic uremic syndrome (aHUS), and age-related macular degeneration (AMD).

144-145. (canceled)
